(19) United States
(12) Patent Application Publication
(10) Pub. No.: US 2019/0076564 A1
(43) Pub. Date: Mar. 14, 2019

(54) SYSTEMS, DEVICES AND METHODS FOR DECONTAMINATING SURFACES OF PHARMACEUTICAL VESSELS AND PHARMACEUTICAL ADMINISTRATION DEVICES

(71) Applicant: Mikael Naygauz, Forrest Hills, NY (US)

(72) Inventor: Mikael Naygauz, Forrest Hills, NY (US)

(21) Appl. No.: 16/100,903

(22) Filed: Aug. 10, 2018

Related U.S. Application Data
(60) Provisional application No. 62/619,795, filed on Jan. 20, 2018.
(60) Provisional application No. 62/619,920, filed on Jan. 22, 2018.
(60) Provisional application No. 62/562,490, filed on Sep. 25, 2017.
(60) Provisional application No. 62/564,933, filed on Sep. 28, 2017.
(60) Provisional application No. 62/612,267, filed on Dec. 29, 2017.
(60) Provisional application No. 62/582,922, filed on Nov. 7, 2017.
(60) Provisional application No. 62/560,195, filed on Sep. 19, 2017.
(60) Provisional application No. 62/563,072, filed on Sep. 26, 2017.
(60) Provisional application No. 62/558,502, filed on Sep. 14, 2017.
(60) Provisional application No. 62/625,334, filed on Feb. 1, 2018.
(60) Provisional application No. 62/644,185, filed on Mar. 16, 2018.
(60) Provisional application No. 62/662,721, filed on Apr. 25, 2018.
(60) Provisional application No. 62/664,933, filed on Apr. 30, 2018.
(60) Provisional application No. 62/676,822, filed on May 25, 2018.
(60) Provisional application No. 62/679,817, filed on Jun. 3, 2018.
(60) Provisional application No. 62/686,602, filed on Jun. 18, 2018.
(60) Provisional application No. 62/686,612, filed on Jun. 18, 2018.
(60) Provisional application No. 62/690,260, filed on Jun. 26, 2018.

(51) Int. Cl. A61L 2/26; A61J 1/20

(52) U.S. Cl. CPC A61L 2/26; B65B 3/003; A61J 1/201; A61J 1/2051

(57) ABSTRACT

The present invention relates, in some embodiments thereof, to systems, devices and methods for decontaminating a surface of one or more vessels. In some embodiments, the systems and devices of the invention include a housing, and a wiping member disposed within the housing being configured to wipe off the surface of one or more vessels, thereby establishing a contaminant-free fluid passageway between vessels.

### CROSS REFERENCE TO RELATED APPLICATIONS

This application claims the benefit of U.S. Provisional Patent Application Nos. 62/545,152 filed on Aug. 14, 2017; 62/558,502 filed on Sep. 14, 2017; 62/560,195 filed on Sep. 19, 2017; 62/562,490 filed on Sep. 25, 2017; 62/563,072 filed on Sep. 26, 2017; 62/564,933 filed on Sep. 28, 2017; 62/566,414 filed on Sep. 30, 2017; 62/582,922 filed on Nov. 7, 2017; 62/612,267 filed on Dec. 29, 2017; 62/619,795 filed Jan. 20, 2018; 62/619,920 filed Jan. 22, 2018; 62/625,334 filed on Feb. 1, 2018; 62/626,686 filed on Feb. 6, 2018; 62/631,729 filed on Feb. 17, 2018; 62/644,185 filed on Mar. 16, 2018; 62/649,483 filed on Mar. 28, 2018; 62/651,000 filed on Mar. 30, 2018; 62/651,149 filed on Mar. 31, 2018; 62/653,356 filed on Apr. 5, 2018; 62/657,806 filed on Apr. 14, 2018; 62/657,808 filed on Apr. 15, 2018; 62/657,809 filed on April 15, 2018; 62/660,885 filed on Apr. 20, 2018; 62/662,721 filed on Apr. 25, 2018; 62/662,743 filed on Apr. 25, 2018; 62/664,933 filed on Apr. 30, 2018; 62/666,866 filed on May 4, 2018; 62/667,593 filed on May 6, 2018; 62/669,948 filed on May 10, 2018; 62/670,833 filed on May 13, 2018; 62/675,058 filed on May 22, 2018; 62/676,822 filed on May 25, 2018; 62/679,817 filed on Jun. 3, 2018; 62/680,576 filed on Jun. 4, 2018; 62/680,974 filed on Jun. 5, 2018; 62/681,884 filed on Jun. 7, 2018; 62/686,602 filed on Jun. 18, 2018; 62/686,612 filed on Jun. 18, 2018; and 62/690,260 filed on Jun. 26, 2018. The contents of the above applications are all incorporated by reference as if fully set forth herein in their entirety.

### FIELD OF THE INVENTION

The present invention relates, in some embodiments thereof, to systems, devices and methods for decontaminating a surface of one or more vessels. In some embodiments, the systems and devices of the invention include a housing, a wiping member disposed within the housing and one or more vessels attached to, or configured to be coupled to the housing, the wiping member configured to decontaminate a surface of the one or more vessels when surfaces of the vessels move from an initial position to a second or final position within the housing.

### BACKGROUND OF THE INVENTION

The medicinal practice routinely involves administration of medical substances, such as, medicaments, fluids, nutritional substances and the alike, to patients or animals. The preparation and/or administration of such medicinal substances typically involves one or more transfers of those substances between pharmaceutical vessels or administration devices (such as, vials, syringes, infusion lines, connectors, etc.). Each such act of transferring substances between vessels or devices exposes the connection interfaces of the vessels and accordingly the medical substances themselves to contaminants present in ambient air or ambient air particles (e.g., bacteria, viruses, funguses, spores, pyrogens, dirt). In addition, connection interfaces are further prone to contaminations due to physical contact of the interfaces, for example, with nonsterile gloves, or devices.

Such contaminations are a major problem in the healthcare setting since contaminants, once invading within medicinal substances, may pose substantial danger if administered intracorporeally to patients.

Typical connection interfaces of pharmaceutical vessels or pharmaceutical administration devices include rubber bungs and/or stoppers covered by a cap and/or seal that can be flicked off and/or are removed prior to usage thereof. These rubber bungs/stoppers are used to allow penetration by a needle attached to a syringe or by other medical connectors. When the cap and/or seal is flicked off and/or removed, the rubber bung and/or stopper is exposed to ambient air and to contaminants present therein. Accordingly, exposure of connection interfaces to ambient air may involve contamination of the interfaces and consequently contamination of a beneficial substance to be provided to a patient.

Existing systems include U.S. D720067; U.S. D717947; U.S. D703812; U.S. D690418; U.S. D639939; U.S. D637713; U.S. Pat. Nos. 9,790,011; 9,775,777; 9,561326; 9,493,281; 9,492,353; 9,309,020; 9,173,816; 9,168,203; 9,162,803; 9,039,672; 8,926,583; 8,827,978; 8,790,330; 8,662,985; 8,657,803; 8,622,985; 8,562,583; 8,545,475; 8,523,838; 8,491,563; 8,480,646; 8,449,521; 8,381,776; 8,336,587; 8,328,772; 8,287,513; 8,225,826; 8,075,550; 8,029,747; 7,998,134; 7,975,733; 7,942,860; 7,867,215; 7,744,581; 7,731,678; 7,387,216; 7,306,584; 6,875,203; 6,729,370; 6,715,520; 6,602,239; 6,409,708; 6,343,629; 6,162,199; 6,113,583; 6,063,068; 5,893,397; 5,876,380; 5,832,971; 5,807,374; 5,746,733; 5,569,235; 5,462,535; 5,405,326; 5,292,318; 5,279,582; 4,944,723; 4,932,947; 4,932,937; 4,919,657; 4,915,701; 4,826,489; 4,673,404; 4,564,054; 3,610,241; 3,605,743; 3,587,575; 3,583,399; 3,578,037; 3,556,099; 3,552,387; 3,406,686; 3,380,450; 3,375,825; 3,342,180; 3,330,282; 3,330,281; 3,306,290; 3,255,752; 3,253,592; 3,076,456; 2,972,991; 2,922,419; US20160262982; US20160038373; US20150209568; US20140183196; US20140016570; US20140007973; US20140000754; US20130184672; US20130006200; US20120209238; US20120209218; US20120203194; US20110284561; US20110186177; US20110125128; US20110108158; US20110098647; US20100249745; US20100198182; US20100152669; US20100147402; US20100036319; US20100004602; US20090057258; US20080312634; US20080223484; US20080171981; US20060276759; US20050215976; US20030199847; US20030187420; US20020130100; US20020115981; US20020099354; ES2577377T3; EP2852367B1; EP2666513; EP2155141B1.

In order to overcome this obstacle, the current medical practice involves swabbing the surface of a connection interface with a disinfecting agent, such as 70% isopropyl alcohol, prior to accessing the connection interface. Other methods include i.v. (intravenous) rooms which are used for the sterile preparation of i.v. medications. Such rooms, to keep medicinal preparations as sterile as possible, are equipped with special instruments including, hoods with air filtration systems (e.g., HEPA filters), ventilation systems and air pressure systems. Additionally, those rooms necessitate that the medical staff working in these rooms are properly garmented, are properly trained, and require aseptic techniques, and employ quality control and validation processes. These systems require regular upkeep by certified personnel and require regular cleaning. These systems are therefore expensive, labor intensive, and require regular maintenance and testing to assure that they are operating effectively. The above described systems and methods are either cumbersome and expensive or inefficient in addressing the problem of reducing/eliminating contaminants on connection interfaces.

Thus, there is a long felt and unmet need for pharmaceutical vessels, devices, systems and/or methods that afford transfer of medical substances in a sterile manner. There is a need for reliable, user friendly and cost-effective solutions allowing contaminant-free engagement of vessels and devices for drug preparation and administration processes.

### SUMMARY OF THE INVENTION

Objects of the invention are achieved by providing systems, devices, vessels and methods for engaging and administering medical substances in a decontaminated manner.

Objects of the invention are achieved by providing systems, devices, vessels and methods which are directed to transferring medical substances in an efficient, user-friendly and sterile manner.

The present invention provides devices and systems that decontaminate connection surfaces of medical or pharmaceutical vessels and thereafter provide for a decontaminated fluid passageway between the vessels.

In a first aspect, the present invention provides a system for the engagement of medical devices, the system comprising: a first medical device; a second medical device; a sliding mechanism positioned on at least one of the first medical device and the second medical device and configured to allow traveling there along of at least one of the first medical device and the second medical device, such that at least one of the first medical device and the second medical device is configured to move from a first position to a second position; and a wiping member disposed on at least one of the first medical device and the second medical device, the wiping member configured to remove contaminants from a surface of at least one of the first medical device and the second medical device at about the time of the movement of the at least one of the first medical device and the second medical device from the first position to the second position.

In one or more embodiments, at least one of the first medical device and/or the second medical device includes a displaceable member, wherein the displaceable member is configured to externally displace from the medical device at about the time at least one of the first medical device and/or the second medical device moves from the first position to the second position. In one or more embodiments, the displaceable member is a surface of the first medical device and/or second medical device. In one or more embodiments, the displaceable member is a seal and/or a cover of an aperture of the first medical device and/or second medical device. In one or more embodiments, the displaceable member seals and/or covers a port and/or an opening of a first medical device and/or a second medical device. In one or more embodiments, the displaceable member is a plastic or a metal alloy. In one or more embodiments, the displaceable member is a polymer. In one or more embodiments, the first medical device and/or the second medical device includes a port. In one or more embodiments, the displaceable member is attached to the first and/or second medical device via an adhesive. In one or more embodiments, the displaceable member is attached to the first and/or second medical device via a rail mechanism disposed on the first medical device and/or second medical device. In one or more embodiments, the displaceable member is disposed on and/or in between the rails of the first and/or second medical devices.

In one or more embodiments, the first medical device is selected from a vial, a bag, a bottle, a syringe, an infusion line, a connector, a filter, a manifold, a spike, a manifold, a connector with multiple openings, a bag port, a bottle port, a vial port, and combinations thereof.

In one or more embodiments, the second medical device is selected from a vial, a bag, a bottle, a syringe, an infusion line, a connector, a filter, a manifold, a spike, a manifold, a connector with multiple openings, a bag port, a bottle port, a vial port, and combinations thereof.

In one or more embodiments, the sliding mechanism comprises a rail, the rail positioned along at least a portion of the first medical device and/or the second medical device. In one or more embodiments, the rail is a plastic material or a polymer. In one or more embodiments, the rail is a metal alloy.

In one or more embodiments, the system includes an engagement mechanism configured to provide an engagement between the first medical device and the second medical device.

In one or more embodiments, the engagement mechanism is located on the first medical device and/or on the second medical device. In one or more embodiments, the engagement mechanism is selected from a thread, a luer, an adhesive tape, a ratchet teeth mechanism, a clasping mechanism and combinations thereof.

In one or more embodiments, the engagement mechanism is attached and/or coupled to the sliding mechanism allowing the engagement mechanism to move from a first position adjacent to the first medical device to a second position. In one or more embodiments, the engagement mechanism forms a unitary structure with the sliding mechanism. In one or more embodiments, the sliding mechanism is a rail mechanism.

In one or more embodiments, the engagement mechanism is further configured to move from the first position to the second position along with the second medical device. In one or more embodiments, the engagement mechanism is further configured to move between a plurality of positions along the medical device. In one or more embodiments, the engagement mechanism is configured to move between a plurality of compartments within the housing of the medical device.

In one or more embodiments, the displaceable member is integrally connected to at least one of the first medical device and the second medical device. In one or more embodiments, the displaceable member is a seal, a cover or a cap.

In one or more embodiments, movement from the first position to the second position comprises removal of a sealing, cover or a cap of at least one of the medical device or medical device ports.

In one or more embodiments, after movement of the second medical device from the first position to the second position, a surface of the second medical device is sterile.

In one or more embodiments, the wiping member is disposed within a housing. In one or more embodiments, the wiping member is disposed within a housing, wherein the housing is attached to the first medical device or second medical device. In one or more embodiments, the housing is integrally attached to the first medical device or the second medical device. In one or more embodiments, the wiping member is located between the first medical device and the second medical device. In one or more embodiments, the wiping member is located on at least one of the first medical device and/or the second medical device. In one or more embodiments, the wiping member is located between compartments of the medical device. In one or more embodiments, a plurality of wiping members is located between the compartments of the housing of the medical device.

In one or more embodiments, the housing has a plurality of compartments and/or chambers disposed within the housing. In one or more embodiments, at least one compartment and/or chamber of the housing has a pressure greater than an adjacent compartment and/or chamber of the housing. In one or more embodiments, at least one compartment and/or chamber of the housing has a pressure less than an adjacent compartment and/or chamber of the housing. In one or more embodiments, at least one compartment and/or chamber of the housing has a pressure greater than ambient air pressure and/or atmospheric pressure. In one or more embodiments, at least one compartment and/or chamber of the housing has a pressure less than ambient air pressure and/or atmospheric pressure.

In one or more embodiments, the wiping member is made of a plastic, an elastomeric material, an absorptive material or a rubber material. In one or more embodiments, the absorptive material is a sponge-like material. In one or more embodiments, the wiping member is covered by a disinfecting and/or sterilizing substance. In one or more embodiments, at least one wiping member rotates on an axle, hinge or the alike. In one or more embodiments, the at least one wiping member is at least partially disposed within the housing. In one or more embodiments, the at least one wiping member is disposed within the housing.

In one or more embodiments, the displaceable member is configured to externally displace from the port at about the time the second medical device moves from the first position to the second position. In one or more embodiments, the displaceable member is configured to externally displace from the port at the time the second medical device moves from the first position to the second position.

In one or more embodiments, the displaceable member on the first medical device is configured to engage a complementary displaceable member on the second medical device and vice versa. In one or more embodiments, the engagement between the displaceable member of the first medical device and the displaceable member of the second medical device is an airtight engagement.

In one or more embodiments, the movement of at least one of the medical devices from the first position to the second position allows a hermetically sealed and essentially sterile engagement between the first medical device and the second medical device.

In one or more embodiments, movement of at least one of the first medical device and/or the second medical device from the first position to the second position allows fluid communication between the first medical device and the second medical device.

In one or more embodiments, the system includes a piercing member configured to pierce a surface of at least one of the first medical device and/or the second medical device.

In one or more embodiments, the piercing member is a needle. In one or more embodiments, the piercing member comprises a hollow conduit which provides for fluidic communication between the first medical device and the second medical device.

In one or more embodiments, the wiping member is covered by a sterilizing substance and/or a disinfecting substance. In one or more embodiments, the sterilizing substance is an antibacterial, an antiviral, and anti-tuberculin, an anti-fungal and combinations thereof.

In another aspect the present invention provides a decontamination device for decontaminating a surface of a vessel, comprising; a medical device; a wiping member located on and/or attached to the medical device, wherein the wiping member is configured to wipe a surface of a vessel.

In yet another aspect the present invention provides a decontamination device for decontaminating a surface of a vessel, comprising: a medical device; a sliding mechanism located on and/or attached to the medical device, wherein the sliding mechanism is configured to allow for the traveling there along of a second medical device.

In yet another aspect the present invention provides a decontamination device for decontaminating a surface of a vessel, comprising: a medical device; a sliding mechanism located on and/or attached to the medical device, wherein the sliding mechanism is configured to allow for the traveling there along of an engagement mechanism; and an engagement mechanism configured to travel along the sliding mechanism, wherein the engagement mechanism is further configured to engage a medical device.

In yet another aspect the present invention provides a device for decontaminating a surface of a medical device, comprising: a medical device; a sliding mechanism located on and/or attached to the medical device, wherein the sliding mechanism is configured to allow for the traveling there along of an engagement mechanism; an engagement mechanism configured to travel along the sliding mechanism, wherein the engagement mechanism is further configured to engage a medical device; and a wiping member configured to wipe a surface of a vessel

In yet another aspect the present invention provides a system for the engagement of medical vessels, the system comprising: a first medical vessel; a second medical vessel; a housing configured to allow traveling there along of at least one of the first and second medical vessel, such that the vessel is configured to move from a first position to a second position within the housing; a wiping member at least partially disposed within the housing, wherein the wiping member is configured to remove contaminants from a surface of the at least one first vessel and the second vessel at about the time of movement of the first vessel and/or the second vessel from the first position to the second position.

In one or more embodiments, wherein the housing is further configured to allow traveling there along of at least one of the first and second medical devices, such that the vessel is configured to move between a plurality of position.

In one or more embodiments, the housing has at least two compartments, at least three compartments, at least four compartments, and at least five compartments.

In one or more embodiments, a rail mechanism is disposed on or in the housing configured to provide the traveling there along of the vessel from the first compartment of the housing, to a second compartment of the housing, to a third compartment of the housing. In one or more embodiments, the rail mechanism is further configured to allow traveling of the vessel to a fourth compartment of the housing and to a fifth compartment of the housing.

In one or more embodiments, the housing comprising a top and a bottom configure to rotate and/or move in relation to each other via a rail mechanism. In one or more embodiments, the rail mechanism is a circumferential rail mechanism.

In yet another aspect the present invention provides a system for the engagement of medical vessels, the system comprising: a first medical vessel attached to a plurality of housings, wherein each of the housings are attached to the first medical vessel via a port or an opening on the first medical vessel and are configured to allow traveling there along of a second medical vessel, such that a surface of the second medical vessel or a portion of the second medical vessel is configured to move from a first position to a second position within the housing, wherein the housing further comprises a wiping member, the wiping member disposed within the housing, the wiping member is configured to remove contaminants from a surface of the second medical vessel at about the time of movement of the first and/or second medical vessel from the first position to the second position.

In yet another aspect the present invention provides a connector for the engagement of medical vessels, the connector comprising: a first housing and a second housing, each configured to engage with and allow traveling there along of a medical vessel, such that the medical vessel may move from a first position to a second position within the housing, wherein the housing further comprises a wiping member disposed within the housing, wherein the wiping member is configured to remove contaminants from a surface of the medical vessel at about the time of movement of the medical vessel from the first position to the second position.

In one or more embodiments, the housing has a side wall, a top wall, and a bottom wall, wherein the top wall and the bottom wall each have an opening adapted for connection to the medical vessels.

In one or more embodiments, the housing has a side wall, and a top wall, wherein the top wall has an opening adapted for connection to the medical vessel and wherein the bottom of the housing is open allowing connection to the medical vessel.

In one or more embodiments, the opening is surrounded by vertical walls extending from the bottom or top wall.

In one or more embodiments, the opening includes internal threads, the internal threads being configured for cooperating with external threads on the first medical vessel and/or the second medical vessel to connect the first medical vessel and/or the second medical vessel to the housing. In one or more embodiments, the opening includes external threads, the external threads being configured for cooperating with internal threads on the first medical vessel and/or the second medical vessel to connect the first medical vessel and/or the second medical vessel to the housing.

In one or more embodiments, the housing has a first non-sterile compartment and a second substantially sterile compartment, and wherein the first position includes locating a surface or a portion of the medical vessel in the first compartment and wherein the second position includes locating the medical vessel in the second compartment.

In one or more embodiments, the wiping member is disposed between the compartments such to isolate hermetically the first and second compartments.

In one or more embodiments, the housing further comprises a third compartment comprising a resealing mechanism for resealing the at least one medical vessel with a cap, a seal or a cover. In one or more embodiments, the third compartment houses a resealing member configured to resealing the at least one medical vessel with a cap, a seal or a cover. In one or more embodiments, the third compartment at least partially houses a resealing member configured to resealing the at least one medical vessel with a cap, a seal or a cover. In one or more embodiments, the housing has a compartment with a resealing member disposed within the compartment. In one or more embodiments, the compartment housing the resealing member is sterile.

In one or more embodiments, the housing further comprises a wiping member disposed between the second compartment and the third compartment.

In one or more embodiments, the housing includes at least one double wiper comprising two or more wiping members disposed one after the other.

In one or more embodiments, the wiping members disposed when spaced apart or when in contact with each other.

In one or more embodiments, the bottom wall of the housing is open allowing traveling there along of the medical vessel. In one or more embodiments, the wiping member is configured to extend from the top wall to the bottom wall of the housing.

In one or more embodiments, the housing further comprises a filter disposed in an aperture in the housing for releasing air from the housing when the medical vessel moves along the housing. In one or more embodiments, the filter is a sterilizing filter. In one or more embodiments, the filter has a porosity of 0.22 microns or less. In one or more embodiments the filter may be replaced by a valve configured to release air or pressure from the housing when the medical vessel moves along the housing. In one or more embodiments, the valve is a one-way valve allowing for the release of air from the interior of the housing to the exterior of the housing while preventing ambient or environmental air from entering the housing.

In one or more embodiments, the housing further comprising a locking member disposed in the housing, the locking member being configured for locking the medical vessel in a first position or a second position. In one or more embodiments, the locking member may be present in a plurality of compartments of the housing. In one or more embodiments, the locking member may be located in a first compartment, in a second compartment, in a third or more compartments of the housing. In one or more embodiments, the locking member may prevent retraction of a medical vessel surface or a portion of a medical vessel from the second position back to the first position.

In one or more embodiments, the housing is manufactured from a material selected from the group consisting of plastic, rigid plastic, flexible plastic, and combinations thereof. In one or more embodiments, the housing is manufactured from a glass material.

In one or more embodiments, the wiping member is comprised of an elastomeric material and/or a rubber material. In one or more embodiments, the wiping member is comprised of a rigid material, flexible material, expandable material, an absorptive material, a sponge-like material and combinations thereof. In one or more embodiments, the wiping member is covered by a sterilizing substance and/or disinfecting substance. In one or more embodiments, at least a portion of the wiping member is covered by a sterilizing substance and/or disinfecting substance. In one or more embodiments, the wiping member has a sterilizing and/or disinfecting substance absorbed by the wiping member.

In one or more embodiments, the sterilizing substance is selected from the group consisting of antibacterial, antiviral, anti-tuberculin, anti-fungal, bactericidal, virucidal, fungicidal, spirodical, bacteriostatic, virustatic, fungistatic and combinations thereof. In one or more embodiments, the sterilizing substance is sodium hypochlorite. In one or more embodiments, the concentration of sodium hypochlorite is 0.55% or higher. In one or more embodiments, the concentration of sodium hypochlorite is below 0.55% concentration. In certain embodiments, sterilizing substances may be on the wiper and/or on components in the housing.

In one or more embodiments, the system is disposable, intended for one-time use, or for repeat use.

In one or more embodiments, the medical vessel is configured to move across the housing via a sliding motion.

In one or more embodiments, the wiping member is configured to partially wipe off the surface of at least one vessel. In one or more embodiments, the wiping member is configured to wipe off the entire surface of at least one vessel.

In one or more embodiments, the system includes a plurality of vessels.

In one or more embodiments, the housing has an engagement mechanism to connect the at least one of the vessels to the housing.

In one or more embodiments, the engagement mechanism is selected from a thread, a luer, a ratchet teeth mechanism, a retention member, a smart-site mechanism, a rail mechanism, a snap-on mechanism, a slide-on mechanism and an adhesive mechanism. In one or more embodiments, the thread is configured for engaging a complementary thread on the at least one vessel to connect the at least one vessel to the housing.

In one or more embodiments, the engagement mechanism positions the surface of the at least one vessel at about same level as an interior wall of the housing. In one or more embodiments, the engagement mechanism positions the surface of the pharmaceutical vessel at the same level as an interior wall of a housing of the system. In one or more embodiments, the engagement mechanism positions the surface of the at least one vessel in a manner that protrudes into the housing. In one or more embodiments, the protruding of the surface of the at least one vessel is a slight/minor protruding.

In one or more embodiments, the protruding of the surface of the at least one vessel is ranges from less than one twelfth of an inch to less than half an inch. In one or more embodiments, the engagement mechanism positions the surface of the at least one vessel in a manner that is below an inner surface of the housing. In one or more embodiments, positioning of the surface of the at least one vessel ranges from less than one twentieth of an inch to less than a half an inch from the inner surface of the housing. In one or more embodiments, the engagement mechanism positions the surface of the at least one vessel slightly below an inner wall/surface of the housing while allowing the wiping member to wipe off the entire surface of the at least one vessel.

In one or more embodiments, the at least one vessel ranges from one vessel to ten vessels, wherein a surface of each of the vessels is configured to be wiped off by the wiping member, wherein each of the vessels are configured to couple with the housing.

In one or more embodiments, the port of the housing has an adhesive configured for engaging the at least one vessel to connect the at least one vessel to the housing. In one or more embodiments, the vessel has an adhesive configured for engaging the housing.

In one or more embodiments, each of the ports is connected to a vessel such that the housing forms an interior which is sealed/isolated from surrounding/environmental air. In one or more embodiments, each of the ports is connected to a vessel such that the housing forms an interior which is hermetically sealed/isolated from surrounding/environmental air. In one or more embodiments, each of the plurality of ports provides airtight engagement between the housing and a vessel.

In one or more embodiments, the housing further comprising at least one port configured for coupling to the first and/or the second vessel, wherein the port provides a connection between the housing and the first vessel and/or the second vessel.

In one or more embodiments, the port is covered by a cover. In one or more embodiments, the port is sealed. In one or more embodiments, the seal is a frangible seal. In one or more embodiments, the seal is a rupturable/breakable seal. In one or more embodiments, the seal is a pierceable seal.

In one or more embodiments, the housing has at least two ports, wherein one of the at least two ports are attached to or integrally attached to one of the first vessel and/or the second vessel.

In one or more embodiments, the housing has at least two ports, wherein one of the at least two ports are permanently/fixedly attached to the first or second vessel. In one or more embodiments, the one of the at least two ports form a unitary structure with a vessel. In one or more embodiments, the housing has at least two ports, wherein one of the at least two ports are attached to or coupled to the at least one vessel at about the time of manufacture and/or distribution of the system.

In one or more embodiments, the port is a container port, bag port, bottle port, vial port, syringe port, infusion line port, connector port, manifold port, a vessel port, a spike port and combinations thereof.

In one or more embodiments, the vessel is selected from a bag, a bottle, a vial, a syringe, an infusion line, a connector, a filter, a manifold, a bag port, a bottle port, and a vial port.

In one or more embodiments, the surface of the first and/or second vessel is flat and smooth. In one or more embodiments, the surface of the first and/or second vessel is rigid. In one or more embodiments, the surface of the first and/or second vessel is not smooth.

In one or more embodiments, the contact between the wiping member and the surface provides enough friction between the surface and the wiping member to wipe off contaminants from the surface of the at least one vessel.

In yet another aspect the present invention provides a housing having a plurality of compartments, wherein at least one of the compartments houses a sterilizing or disinfecting substance. In one or more embodiments, the housing having at least two compartments, at least three compartments, at least four compartments or at least five compartments. In one or more embodiments, the sterilizing substance is present inside at least two compartments, at least three compartments, at least four compartments or at least five compartments.

In one or more embodiments, the housing has at least one compartment having an interior pressure greater than an adjacent compartment of the housing.

In one or more embodiments, the housing has at least one compartment having an interior pressure greater than ambient air pressure. In one or more embodiments, the housing has at least one compartment having an interior pressure greater than atmospheric pressure. In one or more embodiments, the housing has at least one compartment having an interior pressure less than an adjacent compartment of the housing. In one or more embodiments, the housing has at least one compartment having an interior pressure less than ambient air pressure. In one or more embodiments, the housing has at least one compartment having an interior pressure less than atmospheric pressure.

In yet another aspect the present invention provides a device for decontaminating a surface of a vessel, comprising: a housing having a top and a bottom; a rail mechanism configured to provide for the top of the housing to rotate in relation to the bottom of the housing; at least one wiping member disposed within the housing; and at least one opening configured to engage a vessel, wherein when the vessel engages the at least one opening and when a surface of the vessel is moved from an initial position to a second position, the surface of the vessel is decontaminated.

In one or more embodiments, the housing has at least one compartment that remains sterile. In one or more embodiments, the housing has at least two compartments. In one or more embodiments, the housing has at least three compartments. In one or more embodiments, the housing has at least four compartments. In one or more embodiments, the housing has five compartments. In one or more embodiments, the housing has a plurality of compartments.

In one or more embodiments, the housing has at least one compartment which are sterile. In one or more embodiments, the housing has at least two compartment which are sterile. In one or more embodiments, the housing has at least three compartments which are sterile. In one or more embodiments, the housing has at least four compartment which are sterile. In one or more embodiments, the housing has five compartments which are sterile.

In one or more embodiments, the housing has at least one double wiper. In one or more embodiments, the housing has at least two double wipers. In one or more embodiments, the housing has a plurality of double wipers. In one or more embodiments, the housing has a compartment that serves as a fluidic passageway between two vessels.

In one or more embodiments, the housing has a compartment housing a resealing member. In one or more embodiments, the resealing member is a cap or a seal. In one or more embodiments, the compartment housing the resealing member is sterile. In one or more embodiments, the resealing member remains sterile.

In one or more embodiments, the housing is attached to a vessel. In one or more embodiments, the housing is integrally attached to a vessel. In one or more embodiments, the housing forms a unitary structure with a vessel. In one or more embodiments, the top of the housing and the bottom of the housing form an interior compartment that is isolated from ambient/environmental air.

In one or more embodiments, the top of the housing and the bottom of the housing form an interior chamber that is isolated from ambient/environmental air. In one or more embodiments, the opening is sealed/covered.

In one or more embodiments, the device is configured to move the vessel between a plurality of compartments. In one or more embodiments, the device is configured to move the vessel from an initial position, to a secondary position, then to a third position. In one or more embodiments, the device is configured to move the vessel from a third position to a fourth position. In one or more embodiments, the device is configured to move the vessel from a fourth position to a fifth position.

In one or more embodiments, at least one of the compartments of the housing contains a sterilizing or disinfecting substance.

In one or more embodiments, at least one of the compartments of the housing has a pressure greater than an adjacent compartment of the housing. In one or more embodiments, at least one of the compartments of the housing has a pressure less than an adjacent compartment of the housing. In one or more embodiments, at least one of the compartments of the housing has a pressure greater than ambient air or atmospheric pressure. In one or more embodiments, at least one of the compartments of the housing has a pressure less than ambient air or atmospheric pressure.

In one or more embodiments, the rail mechanism is a circumferential rail mechanism. In one or more embodiments, the housing is surface mounted to a wall of a vessel. In one or more embodiments, the housing is flush mounted to a wall of a vessel. In one or more embodiments, the housing abuts a wall of a vessel.

In yet another aspect the present invention provides a device for decontaminating a surface of a vessel, comprising: a housing having a top and a bottom; a hinge mechanism configured to provide for the top of the housing to rotate in relation to the bottom of the housing; at least one wiping member disposed within the housing; and at least one opening configured to engage a vessel, wherein when the vessel engages the at least one opening and when a surface of the vessel is moved from an initial position to a second position, the surface of the vessel is decontaminated.

In one or more embodiments, the housing has at least one compartment that remains sterile. In one or more embodiments, the housing has at least two compartments. In one or more embodiments, the housing has at least three compartments. In one or more embodiments, the housing has at least four compartments. In one or more embodiments, the housing has five compartments. In one or more embodiments, the housing has a plurality of compartments. In one or more embodiments, the housing has at least one compartment which are sterile. In one or more embodiments, the housing has at least two compartment which are sterile. In one or more embodiments, the housing has at least three compartments which are sterile. In one or more embodiments, the housing has at least four compartment which are sterile. In one or more embodiments, the housing has five compartments which are sterile.

In one or more embodiments, the housing has at least one double wiper. In one or more embodiments, the housing has at least two double wipers.

In one or more embodiments, the housing has a compartment that serves as a fluidic passageway between two vessels. In one or more embodiments, the housing has a compartment housing a resealing member. In one or more embodiments, the resealing member is a cap or a seal. In one or more embodiments, the compartment housing the resealing member is sterile. In one or more embodiments, the resealing member remains sterile.

In one or more embodiments, the housing is attached to a vessel. In one or more embodiments, the housing is integrally attached to a vessel. In one or more embodiments, the housing forms a unitary structure with a vessel.

In one or more embodiments, the top of the housing and the bottom of the housing form an interior compartment that is isolated from ambient/environmental air. In one or more embodiments, the top of the housing and the bottom of the housing form an interior chamber that is isolated from ambient/environmental air. In one or more embodiments, the opening is sealed/covered.

In one or more embodiments, the device is configured to move the vessel between a plurality of compartments. In one or more embodiments, the device is configured to move the vessel from an initial position, to a secondary position, then to a third position. In one or more embodiments, the device is configured to move the vessel from a third position to a fourth position. In one or more embodiments, the device is configured to move the vessel from a fourth position to a fifth position.

In one or more embodiments, at least one of the compartments of the housing contains a sterilizing or disinfecting substance.

In one or more embodiments, at least one of the compartments of the housing has a pressure greater than an adjacent compartment of the housing. In one or more embodiments, at least one of the compartments of the housing has a pressure less than an adjacent compartment of the housing. In one or more embodiments, at least one of the compartments of the housing has a pressure greater than ambient air or atmospheric pressure. In one or more embodiments, at least one of the compartments of the housing has a pressure less than ambient air or atmospheric pressure.

In one or more embodiments, the housing is surface mounted to a vessel. In one or more embodiments, the housing is flush mounted to a vessel. In one or more embodiments, the housing abuts a wall of a vessel.

In yet another aspect the present invention provides a method of decontaminating a surface of at least one medical device, the method comprising the steps of: providing a first medical device; providing a second medical device, wherein at least one of the first medical device and the second medical device has a sliding mechanism configured to allow traveling there along of at least one of the first medical device and the second medical device, such that at least one of the first medical device and the second medical device is configured to move from a first position to a second position; providing a wiping member disposed on at least one of the first medical device and the second medical device, the wiping member configured to remove contaminants from a surface of at least one of the first medical device and the second medical device at about the time of the movement of at least one the medical device from the first position to the second position; engaging at least one of the first medical device and the second medical device to the sliding mechanism of complementary medical device; and sliding at least one of the first medical device and the second medical device along the sliding mechanism such that at least one of the first medical device and the second medical device is configured to move from a first position to a second position, wherein the wiping member removes contaminants from a surface of at least one of the first medical device and the second medical device upon the movement of at least one of the first medical device and the second medical device from the first position to the second position.

In one or more embodiments, the wiping member is disposed within the first and/or second medical device. In one or more embodiments, the wiping member is disposed within a port of the first and/or second medical device. In one or more embodiments, the first and/or second medical device having a housing, wherein the wiping member is disposed within the housing.

In yet another aspect the present invention provides a method of decontaminating a surface of at least one vessel, the method comprising: providing a decontamination device having a housing and a wiping member at least partially disposed within the housing and configured to wipe a surface of a vessel; providing at least one vessel having a surface; connecting the decontamination device to the at least one vessel; and moving the vessel surface within the housing, thus, decontaminating the surface of the at least one vessel.

In one or more embodiments, movement from the first position to the second position allows hermetically sealed and essentially sterile engagement between the first medical device and second medical device. In one or more embodiments, movement from the first position to the second position allows for fluid communication between the first medical device and second medical device.

In one or more embodiments, the step of sliding occurs via a sliding mechanism comprising a rail, the rail positioned along at least a portion of at least one of the first medical device and the second medical device.

In one or more embodiments, the step of engaging occurs via an engagement mechanism configured to provide a secure engagement between the first medical device and the second medical device.

In one or more embodiments, the method further comprising providing a displaceable member disposed on at least one of the first medical device and the second medical device, the displaceable member being configured to externally displace from at least one of the first medical device and the second medical device at about the time of movement from a first position to a second position.

In one or more embodiments, movement from the first position to the second position comprises removal of a sealing or a cap member of at least one of the first medical device and the second medical device.

In one or more embodiments, the method further comprising providing a piercing member located on and/or attached to at least one of the first medical device and the second medical device.

In one or more embodiments, the method further comprising piercing a surface of at least one of the first medical device and the second medical device.

In one or more embodiments, the piercing establishes a fluid communication between the first medical device and the second medical device.

In one or more embodiments, the method further comprising applying pressure to at least one surface of the first medical device and the second medical device, wherein application of pressure breaks the frangible surface of at least one of the first medical device and the second medical device, thus establishing a fluidic communication between the first and second medical devices.

In one or more embodiments, the method further comprises movement of a vessel surface between/amongst a plurality of positions. In one or more embodiments, the method further comprises movement of a vessel surface between/amongst a plurality of compartments of the housing.

In one or more embodiments, the method further comprises sliding at least one of said first medical device and said second medical device along said sliding mechanism from a second position to a third position.

In one or more embodiments, the method further comprises resealing the at least one of said first medical device and said second medical device in the third position.

In one or more embodiments, the method further comprises disconnecting the at least one of said first medical device and said second medical device.

In yet another aspect, a syringe tip/opening/aperture is sealed with a puncturable and/or frangible seal. The seal is configured to be wiped off by the wiping member. In one or more embodiments, the syringe tip/opening/aperture may be covered by a seal that is punctured by piercing member of decontamination device or ruptured inside decontamination device. The syringe seal may also be displaceable inside the decontamination device.

In yet another aspect the present invention provides a device for the decontamination of a surface of a vessel, comprising: a housing having a top and a bottom; at least one wiping member disposed within the housing; at least one opening within the housing configured to engage at least one vessel; and a sliding mechanism configured to provide for the sliding of the vessel surface from a first compartment of the housing to at least a second compartment of the housing, wherein when the at least one vessel engages the at least one opening of the vessel, the at least one vessel slide along the sliding mechanism of the device from a first position to at least a second position.

In one or more embodiments, the system includes a third compartment.

In one or more embodiments, the third compartment includes a resealing mechanism for resealing the at least one vessel with a cap, a seal or a cover.

In one or more embodiments, when the at least one vessel slides from the second position to the third position, the at least one vessel interacts with the resealing mechanism to reseal the at least one vessel.

In one or more embodiments, at least one wiping mechanism is located between the first compartment and the second compartment. In one or more embodiments, a second wiping member is located between the second compartment and the third compartment. In one or more embodiments, the second compartment includes a sterilizing/disinfecting substance. In one or more embodiments, the third compartment includes a sterilizing/disinfecting substance. In one or more embodiments, the system includes a fourth compartment and a fifth compartment.

In one or more embodiments, a third wiping member is located between the third compartment and the fourth compartment and a fourth wiping member is located between the fourth compartment and the fifth compartment.

In one or more embodiments, the fifth compartment includes a resealing mechanism for resealing the at least one vessel with a cap, a seal or a cover. In one or more embodiments, the resealing member is disposed within the housing.

In one or more embodiments, the device further comprising a retractable safety mechanism to provide for the one directional movement of a vessel between compartments of the housing so that the vessel cannot retract. In one or more embodiments, the retractable safety mechanism prevents a vessel when attached to the housing from retracting from a second position to a first position. In one or more embodiments, the retractable safety mechanism prevents a vessel when attached to the housing from retracting from a third position to a second position. In one or more embodiments, the retractable safety mechanism prevents a vessel when attached to the housing from retracting from a fourth position to a third position. In one or more embodiments, the retractable safety mechanism prevents a vessel when attached to the housing from retracting from a fifth position to a fourth position. In one or more embodiments, the retractable safety mechanism is applicable to any of the disclosed systems, devices, and methods of this invention.

In one or more embodiments, the device further comprising a piercing member for piercing the surface of at least one vessel. In one or more embodiments, the device further comprising a piercing member safety mechanism configured to prevent the premature piercing of a vessel surface. In one or more embodiments, the piercing member safety mechanism is unlocked by the movement of a vessel from a first compartment to a second compartment of the housing. In one or more embodiments, the piercing member safety mechanism is unlocked by the movement of a vessel from a second compartment to a third compartment of the housing. In one or more embodiments, the piercing member safety mechanism is unlocked by the movement of a vessel from a third compartment to a fourth compartment of the housing. In one or more embodiments, the piercing member safety mechanism is unlocked by the movement of a vessel from a fourth compartment to a fifth compartment of the housing. In one or more embodiments, the piercing member safety mechanism is applicable to any of the disclosed systems, devices, and methods of this invention.

In one or more embodiments, the housing has a plurality of compartments. In one or more embodiments, the housing has detachment safety mechanism configured to prevent the premature detachment of a vessel from a compartment of the housing where resealing occurs. In one or more embodiments, the detachment safety mechanism is unlocked by the movement of a vessel into the compartment where resealing occurs. In one or more embodiments, the detachment safety mechanism is unlocked at about the time of resealing of a vessel surface within the resealing compartment of the housing. In one or more embodiments, the detachment safety mechanism is unlocked at the time of resealing of a vessel surface within the resealing compartment of the housing. In one or more embodiments, the detachment safety mechanism is unlocked after resealing of a vessel surface within the resealing compartment of the housing. In one or more embodiments, the detachment safety mechanism unlocks a wall or sealed aperture of the resealing compartment of the housing at about the time, at the time, and/or after the time of resealing of the vessel surface. In one or more embodiments, the detachment safety mechanism is applicable to any of the disclosed systems, devices, and methods of this invention.

In one or more embodiments, the resealing seal/cover attachment to the vessel is permanent and/or irreversible. In one or more embodiments, the resealing seal/cover attachment to the vessel is temporary, allowing the resealing seal/cover to detach from the vessel surface. In one or more embodiments, the resealing seal/cover is frangible and/or pierceable.

In yet another aspect the present invention provides a device for the decontamination of a surface of a vessel, comprising: a housing having a top and a bottom; at least one wiping member disposed within the housing; at least one opening within the housing configured to engage at least one vessel; and a hinge mechanism configured to provide for the sliding of the vessel surface from a first compartment of the housing to at least a second compartment of the housing, wherein when the at least one vessel engages the at least one opening of the vessel, the at least one vessel may move from a first position to at least a second position.

In one or more embodiments, the system includes a third compartment.

In one or more embodiments, the third compartment includes a resealing mechanism for resealing the at least one vessel with a cap, a seal or a cover.

In one or more embodiments, when the at least one vessel slides or moves from the second position to the third position, the at least one vessel interacts with the resealing mechanism to reseal the at least one vessel.

In one or more embodiments, the at least one wiping mechanism is located between the first compartment and the second compartment. In one or more embodiments, a second wiping member is located between the second compartment and the third compartment. In one or more embodiments, the housing further comprises a fourth compartment. In one or more embodiments, the housing further comprises at least a fifth compartment.

In one or more embodiments, the hinge mechanism is configured to allow for the movement of a vessel surface between a plurality of compartments.

In one or more embodiments, the device having a retractable safety mechanism to provide for the one directional movement of a vessel between compartments of the housing so that the vessel cannot retract. In one or more embodiments, the retractable safety mechanism prevents a vessel when attached to the housing from retracting from a second position to a first position. In one or more embodiments, the retractable safety mechanism prevents a vessel when attached to the housing from retracting from a third position to a second position. In one or more embodiments, the retractable safety mechanism prevents a vessel when attached to the housing from retracting from a fourth position to a third position. In one or more embodiments, the retractable safety mechanism prevents a vessel when attached to the housing from retracting from a fifth position to a fourth position. In one or more embodiments, the retractable safety mechanism is applicable to any of the disclosed systems, devices, and methods of this invention. In one or more embodiments, the second compartment includes a sterilizing/disinfecting substance.

In one or more embodiments, the third compartment includes a sterilizing/disinfecting substance.

In yet another aspect the present invention provides a vessel having a plurality of decontamination devices. In one or more embodiments, at least one decontamination device is an exit port of the vessel and at least one decontamination device is an entry port of the vessel. In one or more embodiments, at least two decontamination devices are entry ports of the vessel. In one or more embodiments, at least three decontamination devices are entry ports of the vessel.

In one or more embodiments, the vessel is a container. In one or more embodiments, the vessel is a syringe. In one or more embodiments, the vessel is a bag. In one or more embodiments, the vessel has a flexible wall. In one or more embodiments, the vessel is a bottle. In one or more embodiments, the vessel has a rigid wall.

In one or more embodiments, the vessel has a plurality of housings, wherein each of the housings has a wiping member disposed within the housings.

In one or more embodiments, the vessel has a plurality of housings, wherein a plurality of wiping member is disposed within the plurality of housings and a plurality of rail or hinge mechanisms are disposed within the housings.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D are front cut view illustrations presenting an exemplary system which includes a housing, a wiping member disposed within the housing and a first vessel coupled to the housing, the housing configured to engage with a second vessel and allow movement of the second vessel from a first position to a second position such that when the vessel moves across the housing the wiping member wipes off the surface of the vessel from contaminants, rendering the vessel surface decontaminated and allowing fluid passageway between the first vessel and the second vessels, according to some embodiments of the invention.

FIGS. 2A-2D are front cut views of the system of FIGS. 1A-1D which further comprises a sliding mechanism in the form of a rail, according to some embodiments of the invention.

FIGS. 3A-3F are front cut view illustrations presenting an exemplary system which may further include a mechanism for releasing or unlocking a displaceable member from the surface of the second vessel, according to some embodiments of the invention.

FIGS. 4A-4E are front cut view illustrations presenting an exemplary system which includes a piercing member for piercing a seal of the second vessel, according to some embodiments of the invention.

FIGS. 5A-5D are front cut view illustrations presenting an exemplary system which includes an engagement mechanism in the form of clamping elements and a filter disposed in an aperture in the housing, for releasing air from the housing when moving the second vessel, according to some embodiments of the invention.

FIGS. 6A-6D are front cut view illustrations presenting an exemplary system which includes an alternative engagement mechanism in the form of threads, according to some embodiments of the invention.

FIGS. 7A-7D are front cut view illustrations presenting an exemplary system which includes an alternative engagement mechanism in the form of ratchet teeth, according to some embodiments of the invention.

FIGS. 8A-8D are front cut view illustrations presenting an exemplary system which includes circumferential wipers for wiping off the sides of a port of a vessel, according to some embodiments of the invention.

FIGS. 9A-9B are front cut view illustrations presenting an exemplary system which includes elongated circumferential wipers for wiping off the sides of a port of a vessel, according to some embodiments of the invention.

FIG. 10 is a front cut view illustration presenting yet a further exemplary system which includes an opened first compartment, according to some embodiments of the invention.

FIG. 11 is a front cut view illustration presenting yet a further exemplary system which includes an opened first compartment with an engagement mechanism in the form of clamping elements, according to some embodiments of the invention.

FIG. 12 is a front cut view illustration presenting yet a further exemplary system which includes an opened first compartment with an engagement mechanism in the form of a thread, according to some embodiments of the invention.

FIG. 13 presents yet a further exemplary system with a vessel connected to a plurality of housings each allowing an engagement with a vessel, according to some embodiments of the invention.

FIGS. 14A-14H are front views of yet a further exemplary system which includes a first compartment for receiving a vessel, a second compartment for fluid transfer between vessels, and a third compartment for resealing and releasing a vessel, according to some embodiments of the invention.

FIGS. 15A-15H are front views of yet a further exemplary system which includes a first compartment for receiving a vessel, a second compartment for fluid transfer between vessels, and a third compartment which includes springs for resealing a vessel, according to some embodiments of the invention.

FIGS. 16A-16E are front views of yet a further exemplary system which includes three compartments and an engagement mechanism in the form of clamping elements, according to some embodiments of the invention.

FIGS. 17A-17E are front views of yet a further exemplary system which includes three compartments and an engagement mechanism in the form of a thread, according to some embodiments of the invention.

FIGS. 18A-18E are front views of yet a further exemplary system which includes three compartments and an engagement mechanism in the form of ratchet teeth, according to some embodiments of the invention.

FIGS. 19A-19D are top perspective views illustrating yet a further exemplary system which includes a housing with a circumferential rail, according to some embodiments of the invention.

FIGS. 20A-20D are bottom perspective views of the system of FIGS. 19A-19D, according to some embodiments of the invention.

FIGS. 21A-21D are bottom perspective views of the system of FIGS. 19-20, which include a double wiper, according to some embodiments of the invention.

FIGS. 22A-22D are bottom perspective views of yet a further exemplary system which includes a hinge mechanism with a first disc and a second disc, for allowing movement of a vessel surface within a housing, according to some embodiments of the invention.

FIGS. 23A-23G are bottom perspective views of yet a further exemplary system which includes a circumferential rail disposed along a first compartment for receiving a vessel, a second compartment for fluid transfer between vessels, and a third compartment for resealing and releasing a vessel, according to some embodiments of the invention.

FIG. 24 is a front cut view illustrating a connector with a double housing, wherein each housing configured to receive a vessel and allow movement of a vessel there along and decontaminate a surface of the vessel, according to some embodiments of the invention.

FIG. 25 is a side cut view of yet a further exemplary system, according to some embodiments of the invention.

FIGS. 26A-26E are front cut views of yet a further exemplary system which includes a double wiper wherein the wiping members are spaced apart, according to some embodiments of the invention.

FIGS. 27A-27E are front cut views of yet a further exemplary system which further includes a double wiper, according to some embodiments of the invention.

FIGS. 28A-28I are front cut views of yet a further exemplary system which further includes two double wipers, according to some embodiments of the invention.

FIGS. 29A-29K are front cut views of yet a further exemplary system which further includes a double wiper wherein the wiping members are spaced apart, according to some embodiments of the invention.

FIGS. 30A-30B are front cut views demonstrating a three-compartment system with a double wiper and an engagement mechanism in the form of a thread, according to some embodiments of the invention.

FIGS. 31A-31B are front cut views demonstrating a three-compartment system with a double wiper and an engagement mechanism in the form of ratchet teeth, according to some embodiments of the invention.

FIGS. 32A-32B are front cut views demonstrating a three-compartment system with a double wiper and an engagement mechanism in the form of fasteners, according to some embodiments of the invention.

FIG. 33 is a side cut view of yet a further exemplary system, wherein a syringe's port includes wings protruding outwardly and allowing engagement with a housing of the system, according to some embodiments of the invention.

FIGS. 34A-34C demonstrate yet a further exemplary system which includes a three-compartment housing, wherein a second compartment includes a sterilizing substance, according to some embodiments of the invention.

FIGS. 35A-35E demonstrate yet a further exemplary system which includes a five-compartment housing, wherein a second compartment and a fourth compartment includes a sterilizing substance, according to some embodiments of the invention.

FIGS. 36A-36C demonstrate yet a further exemplary system which includes a wide wiping member, according to some embodiments of the invention.

FIGS. 37A-37E demonstrate yet a further exemplary system, which include a wide wiping member, according to some embodiments of the invention.

FIG. 38 demonstrates yet a further exemplary decontamination device which includes a housing that can be coupled to a variety of first vessels and second vessels, the housing allows movement of the second vessel and decontamination a surface thereof, according to some embodiments of the invention.

FIG. 39 demonstrates a side view of yet a further exemplary system, according to some embodiments of the invention.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary bedding articles for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention relates to devices, methods and systems allowing an engagement of, and fluid passageway between vessels in a sterile or decontaminated manner. The present devices, methods and systems are particularly useful for medical purposes, wherein sterile preparations of medical substances are routinely required. In one embodiment, the present invention provides systems configured to allow a substantially decontaminated engagement between vessels. In one embodiment, the present invention provides systems configured to decontaminate one or more surfaces of at least one vessel. In one or more embodiments, the systems of the invention allow a substantially decontaminated fluid transfer between one or more vessels.

The invention provides a solution to an unmet and long felt need in medical settings and allows connecting in a sterile manner, two or more vessels. The herein disclosed devices and systems are user friendly, cost effective and abolish the need for complicated and expensive known methods for transferring and administering medical substances.

In an aspect of the invention there is provided a decontamination system for the engagement of medical vessels, the system comprising a first vessel, a second vessel, a sliding mechanism positioned on at least one of the first vessel and the second vessel and configured to allow traveling there along of at least one of the first vessel and the second vessel, such that at least one of the first vessel and the second vessel is configured to move from a first position to a second position; and
a wiping member disposed on at least one of the first vessel and the second vessel, the wiping member is configured to remove contaminants from a surface of at least one of the first vessel and the second vessel at about the time of movement of the medical devices from the first position to the second position.

In one or more embodiments, the devices and systems of the invention include a housing having a side wall, a top wall and a bottom wall, and a wiping member disposed within the housing. In an embodiment of the invention, the top wall and/or bottom wall of the housing have at least one port with port openings for receiving the vessels. The ports and port openings may be formed by vertical walls extending from the top and/or bottom walls, forming an inner channel via which medical substances may be transferred following engagement between, and decontamination of the vessels. Optionally, the bottom wall of the housing includes a wall opening via which a medical vessel may engage with the housing.

In one or more embodiments, at least one of the first vessel and the second vessel is configured to engage with the housing and slide or move there along. A surface of the moveable vessel slides from a first position to a second position within the housing. The vessel may optionally move from a second position to a third position. In one or more embodiments, the movement of the vessel allows a hermetically sealed and essentially sterile engagement between a first vessel and a second vessel. The movement of the vessel is vertically, along a rail disposed within the housing and/or on top of the vessel. In one or more embodiments, the movement of at least one of the vessels from a first position to a second position allows a fluid communication between the first vessel and the second vessel. In one or more embodiments, the movement of the vessel is horizontally, along a rail disposed within or on the housing and/or on a surface of the vessel.

In one or more embodiments, the wiping member is manufactured from or comprises a material including, but not limited to an elastic material, a rigid material, a flexible material, an expandable material, an absorptive material or combinations thereof. In one or more embodiments, the wiping member is manufactured from or comprises an elastomeric material, such as, but not limited to a rubber material. In one or more embodiments, the wiping member is manufactured from or comprises a sponge or a sponge-like material.

In one or more embodiments, the medical vessel or medical vessel surface slides along the housing and passes through the wiping member under great friction, or under a friction sufficient to wipe off any contaminants from the surface of the vessel, such as dirt, bacteria or ambient air particles.

In one or more embodiments, at least one locking member may be disposed in or on the housing to prevent the medical vessel from returning to its initial position.

In an embodiment of the invention, the herein disclosed devices and systems are disposable. In an embodiment of the invention, the herein disclosed devices and systems are non-disposable.

In one or more embodiments, one or more of the surfaces of the vessels is flat and/or smooth. The one or more surfaces may be substantially flat or partially flat or sufficiently flat, allowing efficient decontamination thereof by the wiping member.

In one or more embodiments, the wiping member is covered, at least partially by, or includes, a sterilizing substance. As used herein the term “sterilizing substance” encompasses any substance that can eliminate or reduce the presence of microorganisms, air particles, or any of the alike. In one embodiment, the term “sterilizing substance” is interchangeable with the term “disinfecting substance”. In one or more embodiments, the sterilizing substance is a liquid, a gas, or a semi-solid substance. In one or more embodiments, the sterilizing substance may be, but is not limited to, an antibacterial substance, an antiviral substance (e.g., an anti-HIV agent), an anti-tuberculin substance, an anti-fungal, and combinations thereof. In one or more embodiments, the sterilizing substance may be a bacteriostatic substance, a virustatic substance, a fungistatic substance, a bactericidal substance, a virucidal substance and/or a fungicidal substance. Exemplary sterilizing substances include, without limitation, alcohol, isopropyl alcohol, iodine, lugol's iodine solution and hydrogen peroxide. In one or more embodiments, the sterilizing substance is provided within the housing, optionally within separate compartments within the housing. In one or more embodiments, the sterilizing substance covers at least a portion of at least one wiping member.

In one or more embodiments, the housing may be manufactured from various materials. Optionally, the housing is manufactured from an inert material, thus suitable for various types of pharmaceutical substances. The housing may be made from flexible or from rigid materials. Suitable materials include, without limitation a plastic, a glass, a rigid plastic, a flexible plastic, rubber or combinations thereof.

Various sizes and shapes of the housing are applicable. For example, the housing may have an external most width, length, or height of less than about 100 millimeters (mm). For example, less than about 50 mm, less than about 25 mm, or less than about 10 mm. The housing internal most width may be less than about 9 mm. For example, the housing internal width may be less than about 5 mm, less than about 2.5 mm, or less than about 1 mm. The housing may include one or more compartments which may optionally be separated or divided or isolated by the wiping member or by any other means. In one or more embodiments, the housing includes two compartments, wherein a first compartment is configured to receive a vessel and may be contaminated or non-sterile and a second compartment that is kept sterile allows fluid transfer between two vessels. In one or more embodiments, the sterile compartment can receive a vessel following decontamination of the vessel's surface. In one or more embodiments, the housing includes two compartments wherein a first compartment is configured to receive a vessel and a second compartment is configured to allow fluid transfer between two or more vessels. Optionally, the housing includes three or more compartments. In an exemplary embodiment, the third compartment includes a resealing mechanism and allows resealing or recapping of a vessel. In an exemplary embodiment, the second compartment includes a sterilizing/disinfecting substance. Optionally, the housing includes five compartments. In an exemplary embodiment, the second compartment and fourth compartment include a sterilizing/disinfecting substance. The housing may or may not include a bottom wall. The housing may include a bottom wall which is partially open. Optionally, the housing includes a first compartment which is open at the bottom or which does not include bottom wall. Alternatively, or additionally the first and third compartments may be open at the bottom or may not include a bottom wall. Further optionally, the second compartment may include a bottom wall which may include an opening. Further optionally, all compartments are open at the bottom or may not include a bottom wall. In one or more embodiments, the housing has a plurality of compartments. In one or more embodiments, the housing may abut a wall of a container, may be flush mounted to a wall of a container, and/or be surface mounted to a wall of a container. In one or more embodiments, the container may be a bag, a bottle or a vial. In one or more embodiments, the housing may be integrally attached to the container. In one or more embodiments, the housing may form a unitary structure with the container. In one or more embodiments, the housing may be attached and/or integrally attached to a syringe. In one or more embodiments, the housing may form a unitary structure with the syringe. In one or more embodiments, the housing may be attached and/or integrally attached to an infusion line. In one or more embodiments, the housing may form a unitary structure with the infusion line. In one or more embodiments, the infusion line may be an intravenous line. In one or more embodiments, the infusion line may be an arterial line.

The vessel may move from a first position in which a vessel surface is located within the first compartment to a second position in which the vessel surface is located in the second compartment. Optionally, the housing includes a third compartment and the vessel surface is moveable from the second position to a third position. Optionally, the housing includes a fourth compartment, or a fifth compartment and the vessel surface is moveable from a third position to a fourth position and/or a fifth position.

In one or more embodiments, the housing is conveniently light presenting a weight of no more than about 100 grams (gr). For example, less than about 50 gr, less than about 25 gr, less than about 15 gr, less than about 10 gr, or less than about 5 gr.

The hosing may be transparent allowing visualization of its interior, or opaque.

The housing may be a closed chamber which forms a tight, optionally, airtight connection between the vessels. The housing may be a partially closed chamber. In one or more embodiments, when the ports or openings are connected to vessels or when the ports are covered by a cover or a seal, the housing may form an interior which is hermetically sealed/isolated from surrounding/environmental air.

The housing may optionally include one or more aperture(s), optionally covered by a filter, to thereby allow releasing air from the housing when a medical vessel passes through the wiping member. In one or more embodiments, the filter may be a sterilizing filter. In one or more embodiments, the filter may have a porosity of 0.22 microns or less. In one or more embodiments, a plurality of compartments of the housing have a filter in a wall of the compartments. Optionally, the one or more aperture(s) may be covered by a valve, to thereby allow releasing air from the housing when a medical vessel passes through the wiping member. In one or more embodiments, the valve may be a one-way valve.

In one or more embodiments, the system of the invention is provided when the housing is already connected to one or more medical vessels. In accordance with this embodiment, the one or more medical vessels may be integrally manufactured with the housing and provided or distributed to consumers as such. In accordance with this embodiment, the system may be configured to allow a connection to additional one or more vessels. Alternatively, the system of the invention is provided without any attachment to a vessel and is configured to connect a plurality of vessels and decontaminate the vessels.

In one or more embodiments, the at least one vessel ranges from one vessel to ten vessels, wherein a surface of at least one or each of the vessels is configured to be wiped off by the wiping member.

In one or more embodiments, the system includes one or more ports configured to attach a medical vessel.

In one or more embodiments, the systems and devices of the invention include a sliding mechanism positioned on at least one of a first medical vessel and a second medical vessel and is configured to allow traveling there along of at least one of the first medical vessel and the second medical vessel, such that at least one of the first medical vessel and the second medical vessel is configured to move from a first position to a second position. In one or more embodiments, the systems and devices of the invention include a sliding mechanism positioned on or within a housing thereby allowing traveling or sliding there along of at least one of the first medical vessel and the second medical vessel, such that at least one of the first medical vessel and the second medical vessel is configured to move from a first position to a second position. Optionally, in one or more embodiments provides for the travelling a vessel between a plurality of positions, such as from a second position to a third position, from a third position to a fourth position, and from a fourth position to a fifth position. In one or more embodiments, the sliding mechanism allows only unidirectional sliding. In one or more embodiments, the sliding mechanism allows for the vessel to retract/return to a previous position. Various types of sliding mechanisms are contemplated as long as they can be connected or engage a vessel and afford traveling there along and allow decontamination of a surface of the vessel. The sliding mechanism may include a rail. The rail may be optionally made from a plastic or polymetric material, but a metallic material or glass material is also applicable. In one embodiment, the rail is manufactured from a plastic material. In one or more embodiments, the plastic material is rigid. In one or more embodiments the vessel surface is positioned on a surface of the rail. In one or more embodiments, the vessel surface is positioned between the rails of the rail. In one or more embodiments, the vessel surface is positioned between a portion of the rails. In one or more embodiments, the rail may be a circumferential rail. In one or more embodiments, the sliding mechanism may be a hinge mechanism.

In one or more embodiments, the housing and/or medical vessel has an engagement mechanism configured or adapted to connect the at least one vessel to the housing. In one or more embodiments, the herein disclosed systems include an engagement mechanism configured to provide an engagement between first and second medical vessels. Various types of engagement mechanisms may be applicable and are contemplated. For example, the engagement mechanism is selected from, without limitation, a thread, a luer, a luer-lock, a luer-slip, a smart-site, a ratchet teeth mechanism, a retention member, a fastener, a clamping element, a clamping rail, clamping members or arms, and an adhesive mechanism, and combinations thereof. The thread and the complementary thread may be a luer, an adhesive, a ratchet teeth mechanism, and combinations thereof. In an embodiment of the invention, the engagement mechanism positions the surface of the at least one vessel at about same level as an interior wall of the housing. In an embodiment of the invention, the engagement mechanism positions the surface of the pharmaceutical vessel at the same level as an interior wall of a housing of the system. In an embodiment of the invention, the engagement mechanism positions the surface of the at least one vessel in a manner that protrudes into the housing. In an embodiment of the invention, the protruding of the surface of the at least one vessel is a slight/minor protruding. In an embodiment of the invention, the protruding of the surface of the at least one vessel ranges from less than one twelfth of an inch to less than half an inch. In an embodiment of the invention, the engagement mechanism positions the surface of the at least one vessel in a manner that is below an inner surface of the housing. In an embodiment of the invention, the positioning of the surface of the at least one vessel ranges from less than one twelfth of an inch to less than a half an inch from the inner surface of the housing. In an embodiment of the invention, the engagement mechanism positions the surface of the at least one vessel slightly below an inner wall/surface of the housing while allowing the wiping member to wipe off the entire surface of the at least one vessel. In one or more embodiments, the surface of the at least one vessel is positioned at the same level as an inner surface of a housing wall.

In one or more embodiments, the herein disclosed invention allows transferring medical substances in a contaminant-free, or in a substantially contaminant-free manner.

In one or more embodiments, the herein disclosed invention affords an engagement of vessels in a contaminant-free, or in a substantially contaminant-free manner.

In one or more embodiments, the herein disclosed invention provides a fluidic passageway or communication between vessels in a contaminant-free, or in a substantially contaminant-free manner.

In one or more embodiments, the herein disclosed invention, allows to substantially decrease the chances to introduce contaminants within a medical substance, when preparing medical substances for administration to patients.

In one or more embodiments, the herein disclosed invention, allows for the air-free engagement between at least two vessels.

As used herein the term “substances” refers to various types of materials that should be kept sterile. The substances may be liquid, solid, gelatinous, semi-solid, or gas. In one or more embodiments, the substances are “medical substances”. As used herein the term “medical substances” refers to and encompasses any of the various pharmaceutical drugs, medical drugs, fluids, nutritional products, solid powders, suspensions, liquids and the like. In an embodiment of the invention the term “medical substance” is interchangeable with the term “pharmaceutical substance”. In one or more embodiments medical substances refers to and encompasses sterile injectable substances intended for intracorporal administration to persons in need or animals.

As used herein, the term “contaminant-free” is interchangeable with the term “sterile”, “disinfected”, and “decontaminated”. The term refers to substances that are free or substantially free of ambient air particles and/or pathogens, including but not limited to bacteria, viruses, funguses, spores, dirt and/or pyrogens. Typically, when less or no air is introduced within medical substances, the chances of contamination by pathogens, such as, bacteria, viruses, funguses, spores, pyrogens or the alike is completely abolished or significantly reduced.

As used herein, the term “substantially contaminant-free” means significantly less ambient air present when transferring medical substances with the herein disclosed vessels and systems, as compared to comparable conditions for transferring medical substances without the herein disclosed vessels and systems.

As used herein the term “ambient air particles” is interchangeable with the term “environmental air particles” and refers to air particles present in a non-filtered environment. The air particles may contain dirt, such as dust. Alternatively, or additionally, the air particles may contain pathogens or other microorganisms, or any of the alike. For example, air can be purified by filters, such as a High Efficiency Particulate Air (HEPA) filter.

As used herein the term “vessel” refers to any device utilized for containing, transferring or administering substances as herein disclosed. In one or more embodiments, the vessels may be used for containing medical substances. In an embodiment of the invention, the vessel is a medical vessel. In one or more embodiments, the term “medical vessel” is interchangeable with the term “pharmaceutical vessel”. In an embodiment of the invention, the vessel is a medical device. In an embodiment of the invention, the vessels are used for, and adapted to allow connection to another vessel. The vessel may have a vessel's port via which medical substances may pass towards another vessel. In an embodiment of the invention, the vessel is utilized for containing a medical substance. Various types of vessels are contemplated. The vessel may be selected, without limitation, from a vial, a bag, a chamber, a bottle, a syringe, an infusion line, an administration line, a connector, a filter, a spike, a manifold, a bag port, a bottle port, a vial port and the alike. In an embodiment of the invention, the term vessel further encompasses elements that can be used to connect between vessels. In accordance with this embodiment, the vessel may be selected, without limitation, from a connector, a connector with multiple openings, a connector with multiple ports, a syringe, an infusion line, a tubing, a syringe, a filter, a port and a manifold.

As used herein, the term “connection interface” encompasses any surface, layer plane or the alike that can be attached to a vessel. The term may encompass a structure that can be coupled to a vessel and that can engage with a complementary connection interface. In an embodiment of the invention, the connection interface may seal or cover an aperture of a vessel. In an embodiment of the invention, the connection interface may seal or cover an opening for fluid passageway of a vessel.

As used herein the term “fluid communication” refers to two or more vessels in which substances may pass therethrough either directly or indirectly. The fluid communication may occur via a fluid passageway that allows for the flow/transfer of substances. In an embodiment of the invention, fluid passageway between vessels is established when any seals or covers of the ports of the herein disclosed systems and/or of the vessels are open. Optionally, the vessels and/or ports of the systems are provided with seals/covers and those seals/cover become open upon piercing thereof, for example, by a piercing member. Optionally, the vessels and/or ports of the systems are provided with seals/covers and those seals/cover are opened upon uncovering or removing thereof by a user. Optionally, one or more of the seals/covers disclosed herein take the form of a film, e.g., a thin film. Optionally, one or more of the seals/covers disclosed herein are fragile, allowing piercing thereof with a sharp instrument (i.e., a piercing member). The piercing member may optionally be a needle, for example, a hollowed needle, but other sharp elements are contemplated. Optionally, one or more of the seals/covers disclosed herein are frangible, allowing for the rupturing/breaking of the seals/covers with the application of sufficient force by a user. Optionally, one or more of the vessels includes a displaceable member in the form of a seal or a cap. The displaceable member may be made from various materials, such as, but no limited to a plastic material or a metal material, such as a metal alloy. The displaceable material may be in the form of a foil, or a membrane or a layer. The foil, membrane or layer may be frangible. The displaceable member may be integrally connected to a medical vessel or to a port thereof and may be configured to externally displace from the medical vessel or port at the time the medical vessel moves from the first position to the second position. In one or more embodiments, the displaceable member is configured to engage a complementary displaceable member on a complementary medical vessel. In one or more embodiment, such an engagement is an airtight engagement.

In an embodiment of the invention, the herein disclosed devices, systems and methods allow fluid communication in a contaminant-free, or in a substantially contaminant-free manner. For example, between three or more, four or more, five or more or six or more vessels. In an embodiment of the invention, the herein disclosed devices, systems and methods allow fluid communication between three, four, five six, or seven vessels. For example, the devices, systems, and methods of the invention allow fluid communication between two bags, between a syringe and a bag, between a connector and a bag, between a container and a bag, between a vial and a bag, between a syringe and a vial, between a syringe and a container, between a connector and a container, between a connector and a vial, between a connector and a bag, between an infusion line and a bag, between an infusion line and a syringe or between a syringe and a connector.

In an embodiment of the invention, the herein disclosed device has a plurality of compartments/chambers, wherein there is a pressure differential between at least two of the compartments/chambers. In one or more embodiments, at least one compartment/chamber of the housing has a higher pressure than an adjacent compartment/chamber of the housing. In one or more embodiments, at least one compartment/chamber of the housing has a lower pressure than an adjacent compartment/chamber of the housing. In one or more embodiments, at least one compartment/chamber of the housing has about the same pressure as an adjacent compartment/chamber of the housing. In one or more embodiments, at least one compartment/chamber of the housing has the same pressure as an adjacent compartment/chamber of the housing. In one or more embodiments, at least one compartment chamber of the housing has a pressure greater than or less than ambient air pressure or atmospheric pressure. In one or more embodiments, the pressure of at least one compartment/chamber of the housing is the same as ambient air or atmospheric pressure. In one or more embodiments, the pressure differential between compartments may be at least 0.02 i.w.c. (inches water column). In one or more embodiments, the pressure differential between compartments is at least 0.1 i.w.c. In one or more embodiments, the pressure differential between compartments is between 0.01 i.w.c. and 1 i.w.c. In one or more embodiments, the pressure differential between compartments may be at least negative 0.05 i.w.c. In one or more embodiments, the pressure differential between compartments is between negative 0.04 i.w.c. and negative 0.01 i.w.c. As used herein, the term “i.w.c.” encompasses the definition as provided or accepted by the United States Pharmacopeia, The American Society of Health-Systems Pharmacists, The American Pharmacists Association or other nationally or internationally recognized organizations.

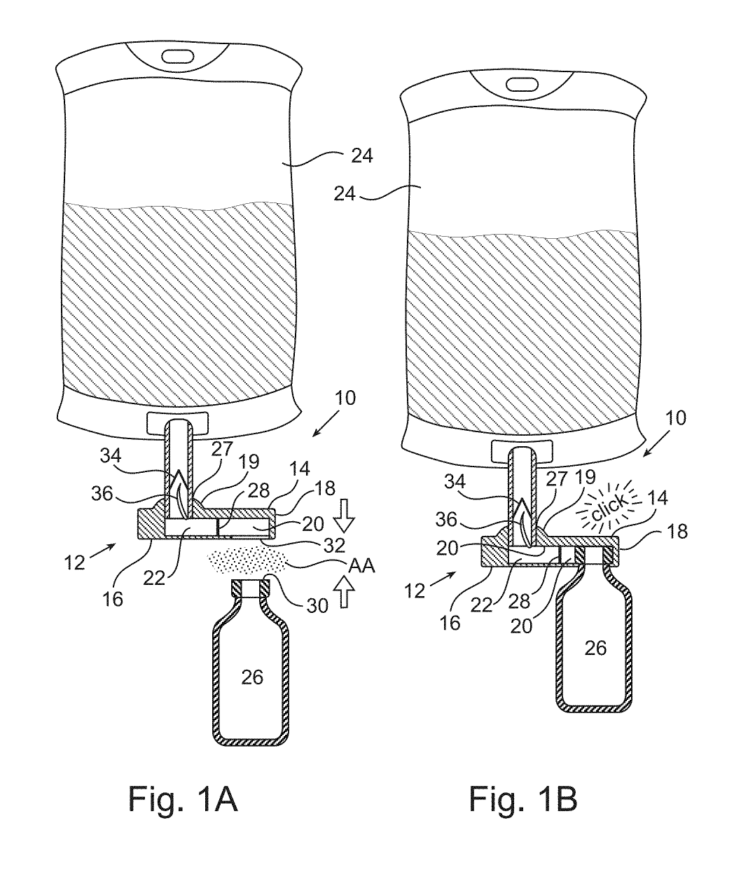

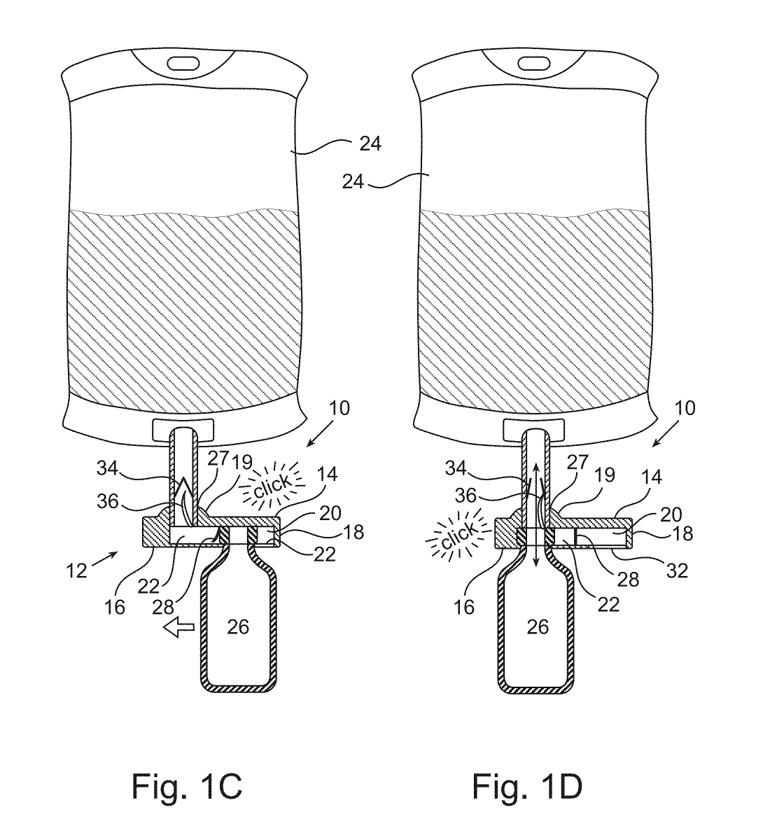

Referring now to the drawings, FIGS. 1A-1D demonstrate system 10 for a decontaminated engagement of medical vessels. System 10 includes a housing 12 with a top wall 14, a bottom wall 16 and a side wall 18. Housing 12 defines a lumen which is divided to at least two compartments, being a first non-sterile compartment 20 and second sterile compartment 22. The housing 12 further optionally comprises vertical side walls 19 extending from top wall 14 and defining top opening 27. Housing 12 further comprises a bottom opening 32 which in this case is not surrounded by vertical walls. Top opening 27 is configured to receive vessel 24 and bottom opening 32 is configured to receive vessel 26. Optionally, system 10 is provided when housing 12 is connected already to, optionally integrally manufactured with, vessel 24. Opening 27 may be covered by a cover (not shown) providing a sealed system 10 prior to an engagement with vessel 24. Alternatively, or additionally opening 32 may be covered by a cover (not shown) providing a sealed system 10 prior to an engagement with vessel 26. Openings 27 and 32 are sized and shaped to allow connection to vessels 24 and 26, respectively, in a tight, optionally, in an airtight manner. Second medical vessel, being vial 26 is configured to be able to attach housing 12 and move there along, thereby moving from a first position (shown in FIG. 1B) to a second position (shown in FIG. 1D). It is to be noted that an infusion bag 24 and a vial 26 are shown, but any type of pharmaceutical/medical vessel could be used and is herein contemplated, such as a bottle, an infusion line, a connector, a filter, a manifold, a syringe, etc. Housing 12 further includes a wiping member 28 disposed within the housing 12, between first compartment 20 and second compartment 22. The wiping member 28 is configured to decontaminate the surface 30 of vessel 26. Wiping member 28 downwardly extends from the inner surface of top wall 14 to the inner surface of bottom wall 16, thus allowing a substantially isolated compartments 20 and 22. Wiping member 28 is constructed such that it touches the inner surface of bottom wall 16, therefore maintaining compartment 22 sterile. System 10 further includes a valve 34, optionally a one way, or a two-way valve, that is closed prior to any decontamination action made by wiping member 28 (FIGS. 1A and 1B). Valve 34 may be operable via valve unlocking mechanism 36 which allows opening of valve 34 at about the time, or at the time of longitudinal alignment of vessel 26 with vessel 24 (FIG. 1D).

FIG. 1A demonstrates the herein disclosed system 10, which includes housing 12 attached to, optionally integrally manufactured with, first medical vessel 24. A second medical vessel 26 is to be attached to the housing 12 from bottom opening 32 disposed at bottom wall 16. Ambient air particles AA surround vessel 26 of system 10. FIG. 1B demonstrates vessel 26 following engagement with housing 12. A sound may be heard, optionally a click sound, upon the engagement of vessel 26 with the housing 12. FIG. 1C demonstrates vessel 26 that moves from the first position to a second position, or from first non-sterile compartment 20 to second substantially sterile compartment 22 within housing 12. Upon movement to second compartment 22, the surface 30 of vessel 26 is wiped off by wiping member 28, allowing removal of contaminants from the surface 30, such as dirt or bacteria. Optionally great friction, or a friction sufficient to wipe off any contaminants is formed between vessel 26 and wiping member 28. Wiping member 28 is configured to remove contaminants from a surface 30 of second medical vessel 26 at about the time of movement of vessel 26 from the first position to the second position. A sound may be heard, optionally a click sound, upon the movement of vessel 26 to the second compartment 22 and/or upon wiping off the surface 30. FIG. 1D demonstrates vessel 26 when positioned at second compartment 22. Vessel 26 is longitudinally aligned with vessel 24. A fluid passageway is formed between vessels 24 and 26 and valve 34 is opened upon unlocking thereof, optionally via valve unlocking mechanism 36. A sound may be heard, optionally a click sound, at about the time, or at the time of the establishment of fluid passageway between vessels 24 and 26 and/or at about the time or at the time of opening valve 34.

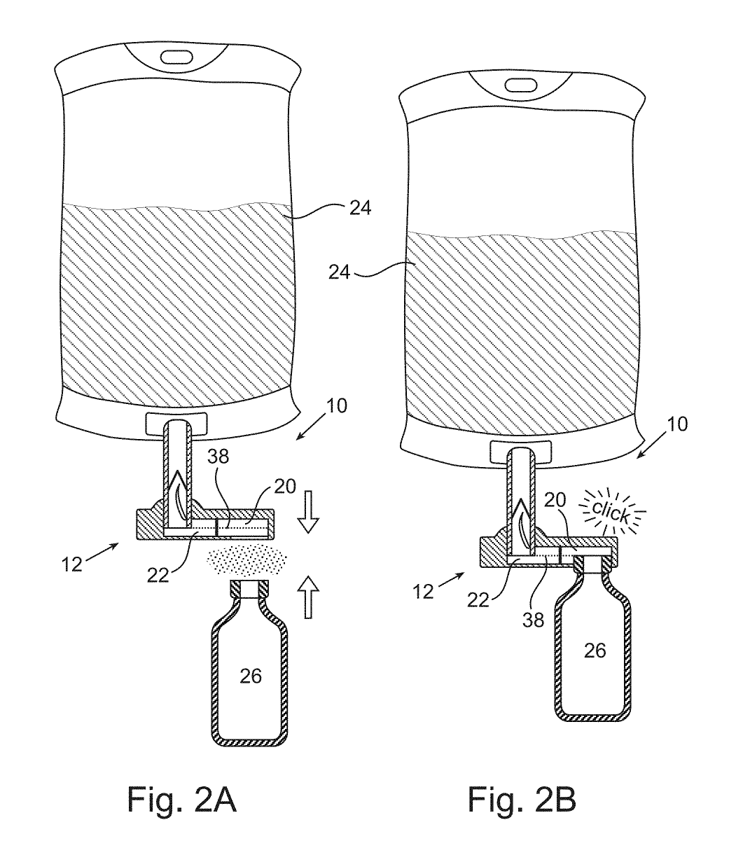

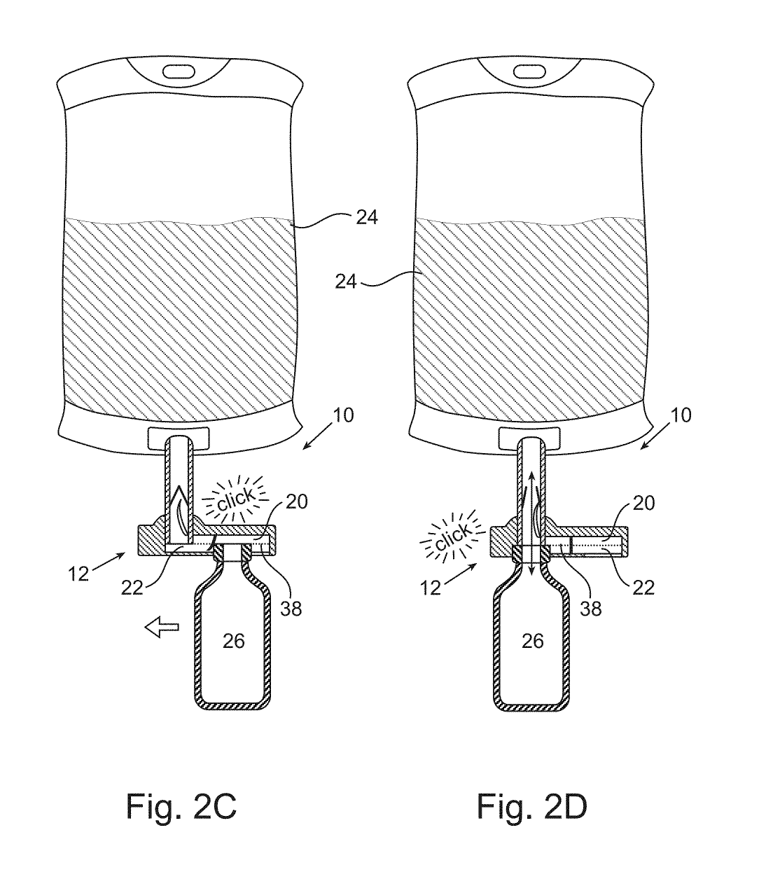

Referring now to FIGS. 2A-2D, system 10 is demonstrated. System 10 may further include a sliding mechanism comprising rail 38 positioned within housing 12, optionally along a portion of a vertical axis, or vertically crossing first and/or second compartments 20 and 22, respectively. Optionally, rail 38 may be positioned on the exterior (not shown) of housing 12. Rail 38 is configured to allow traveling there along of vessel 26, such that it may move from a first compartment 20 (as shown in FIG. 2B) to second compartment 22 (as shown in FIG. 2D). As will be described in greater details below, vessel 26 may optionally or additionally include a rail allowing movement of the vessel along housing 12.

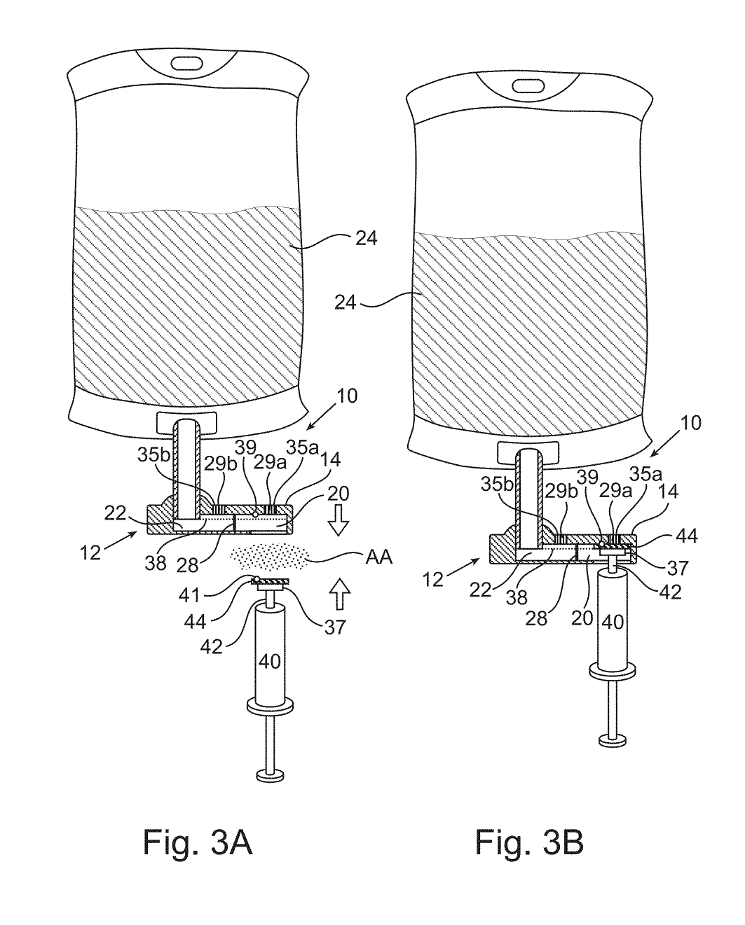

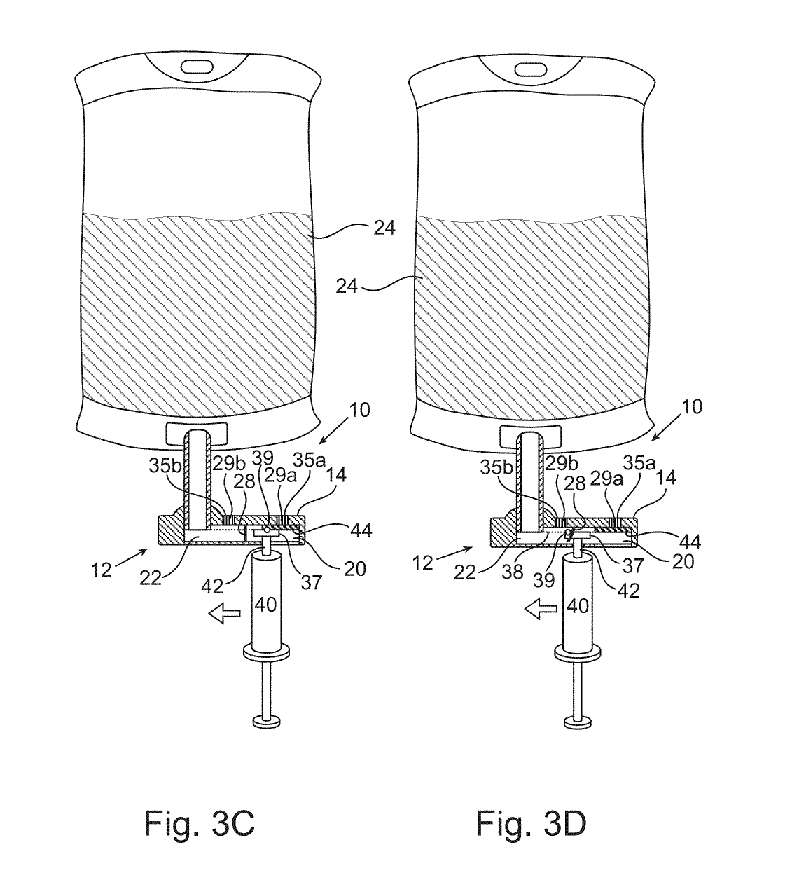

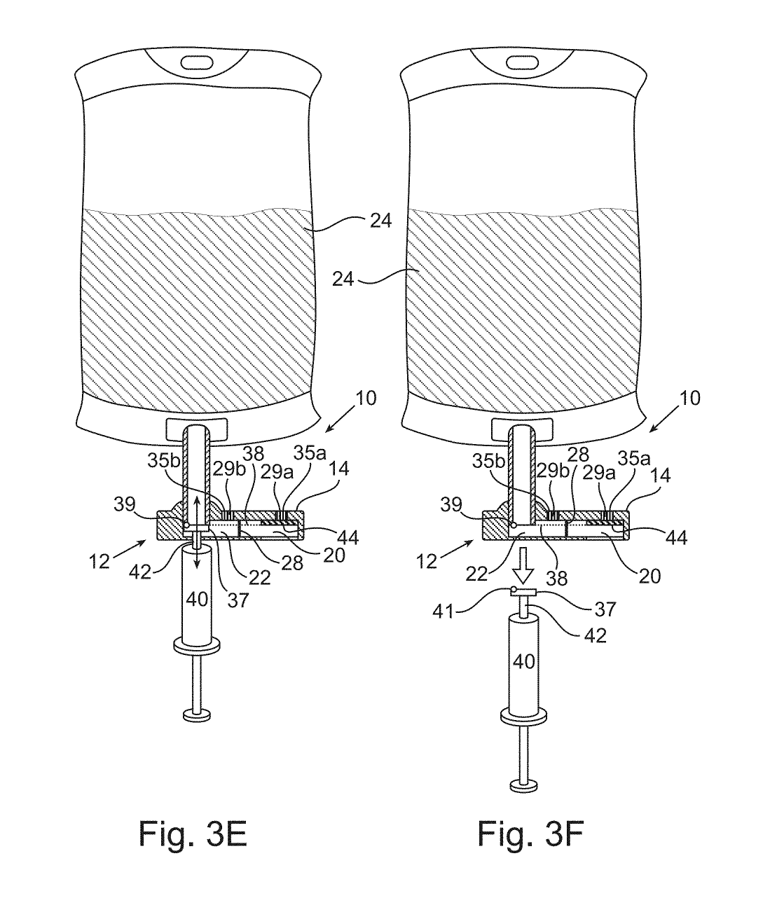

Referring now to FIGS. 3A-3F, a further embodiment of system 10 is shown wherein it further contains filters 29a and 29b disposed in or along apertures 35a and 35b in the housing 12. As demonstrated herein, filter 29a is positioned on top wall 14, above first compartment 20 and filter 29b positioned on top wall 14 above second compartment 22. Filters 29 configured to allow for releasing air from the housing 12 when moving vessel 40 along housing 12. Accordingly, air pressure that can be built up during movement of vessel 40 can be prevented or released. Filter 29 are located and devised so as to allow the pressurized air to pass there through, without allowing any contaminants inside. Filter 29 may have pores having a diameter of less than a few microns, such as 5 microns, so that air can be released but contaminants cannot enter. Filter 29 may be a sterilizing filter. Filter 29 may have a porosity of 0.22 microns or less. Optionally, filter 29 may be a valve, wherein the valve may be a one-way valve.

System 10 is demonstrated when a syringe 40 is to be engaged with vessel 24. Syringe 40 includes a vessel's rail 37 disposed on top or around syringe port 42, and a displaceable member 44 disposed on top of vessel's rail 37. The displaceable member 44 is configured to externally displace from the syringe port 42 following engagement of syringe 40 at first compartment 20. Optionally, displaceable member 44 externally displaces from the port 42 at about the time syringe 40 moves from the first position (FIG. 3B) to the second position (FIG. 3C). As shown herein, system 10 may therefore further comprise at first compartment 20, a releasing member 39 which cooperates or interacts with a complementary releasing member 41 positioned on displaceable member 44, to thereby allow displacement of displaceable member 44 from syringe 40. Optionally, releasing member 41 may be positioned on vessel's rail 37), syringe port 42 or on syringe 40.

FIG. 3A demonstrates syringe 40 and bag 24 when spaced apart and when AA surround them. At FIG. 3B syringe 40 engages with first compartment 20 of housing 12. Displaceable member 44 is attached at the inner surface of top wall 14 and releasing member 39 interacts with complementary releasing member 41 to thereby allow displacement of displaceable member 44 from syringe port 42. At FIG. 3C syringe 40 is shown when moving along the housing 12 towards second compartment 22. Displaceable member 44 displaces from a surface of syringe 40 and maintained attached to the inner surface of top wall 14. FIG. 3D shows a surface of syringe 40 being wiped off by wiping member 28. FIG. 3E shows syringe 40 when positioned at second compartment 22 of system 10. A fluid passageway is formed between vessel 24 and vessel 40. FIG. 3F shows detachment of syringe 40 from second compartment 22.

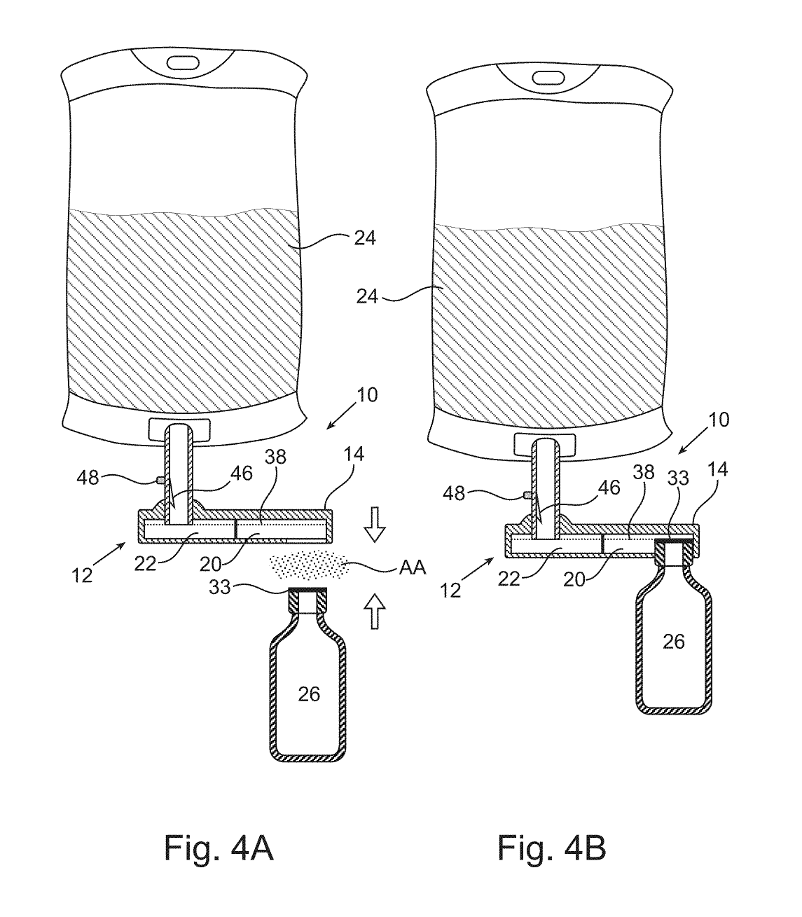

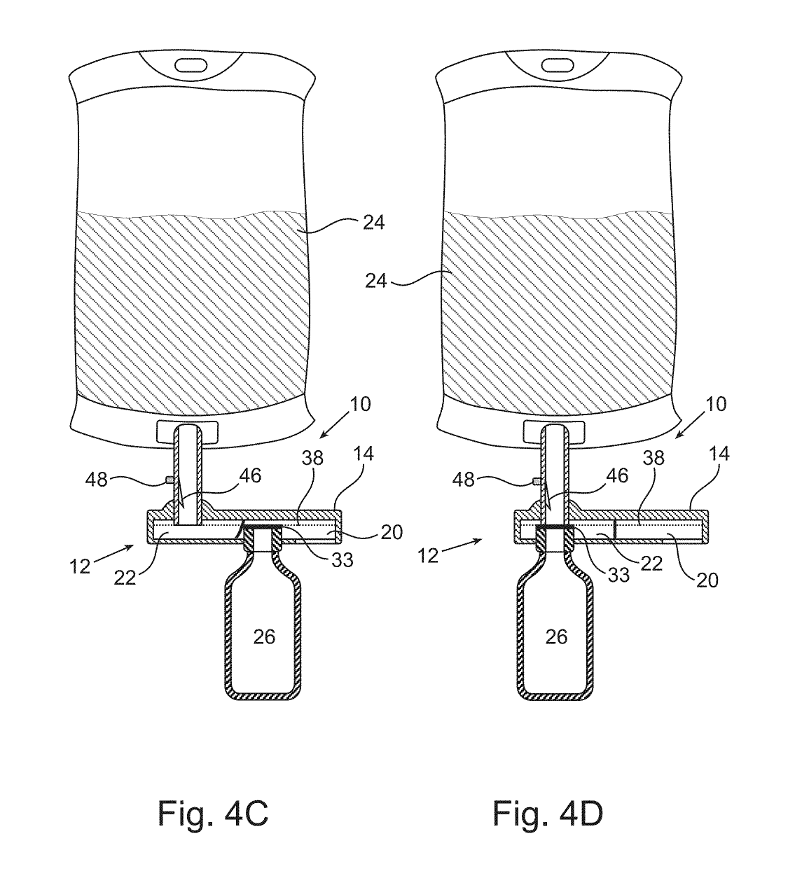

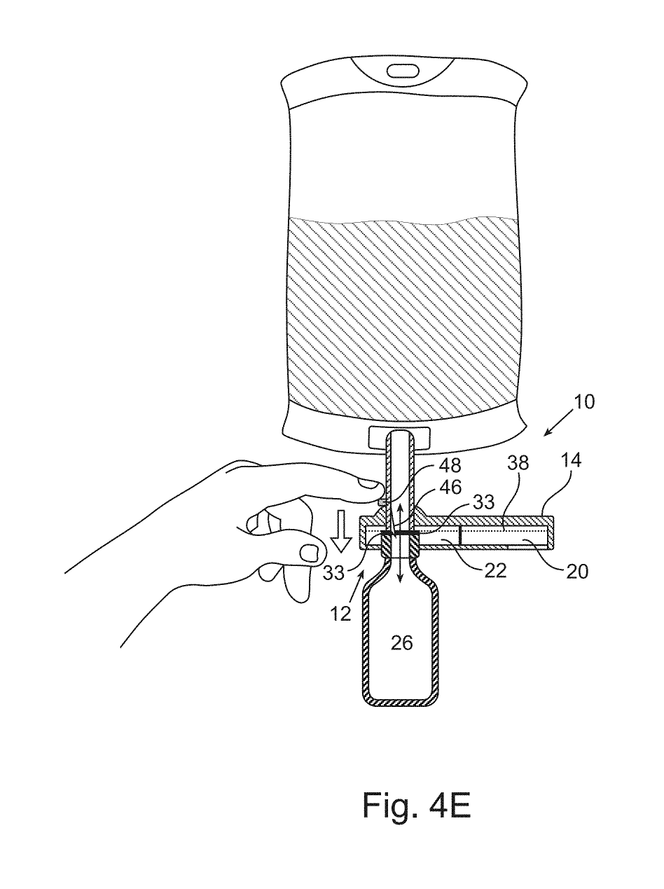

FIGS. 4A-4E demonstrate system 10 which includes a piercing member 46 and a piercing member actuator 48. Piercing member 46 is configured to pierce a seal 33 of second medical vessel 26. Piercing member 46 may be a needle, optionally, a hollowed needle, but various types of piercing members are contemplated, such as members that possess a sharp edge. FIG. 4A shows vessel 26 and system 10 when spaced apart and AA surround them. At FIG. 4B vessel 26 engages housing 12 from the first compartment 20. FIG. 4C shows vessel 26 which slides along rail 38 until reaching second compartment 22 and piercing member 46 is being actuated to pierce sealing 33 of vessel 26 and establish a fluid communication with bag 24. FIG. 4E demonstrates a user that actuates, via piercing member actuator 48, piercing member 46 to pierce sealing 33 of second medical vessel 26. In an alternative embodiment, piercing member actuator 48 is actuated automatically by the motion of vessel 26 to move piercing member 46 downwardly at about the time of movement of vessel 26, and/or at the time the vessel 26 is wiped off.

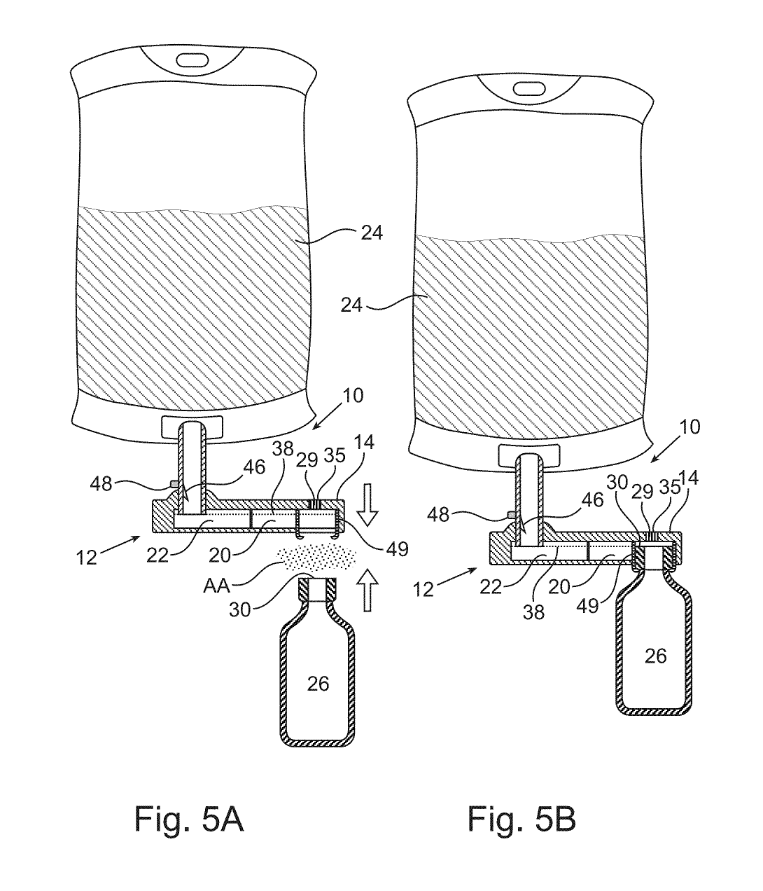

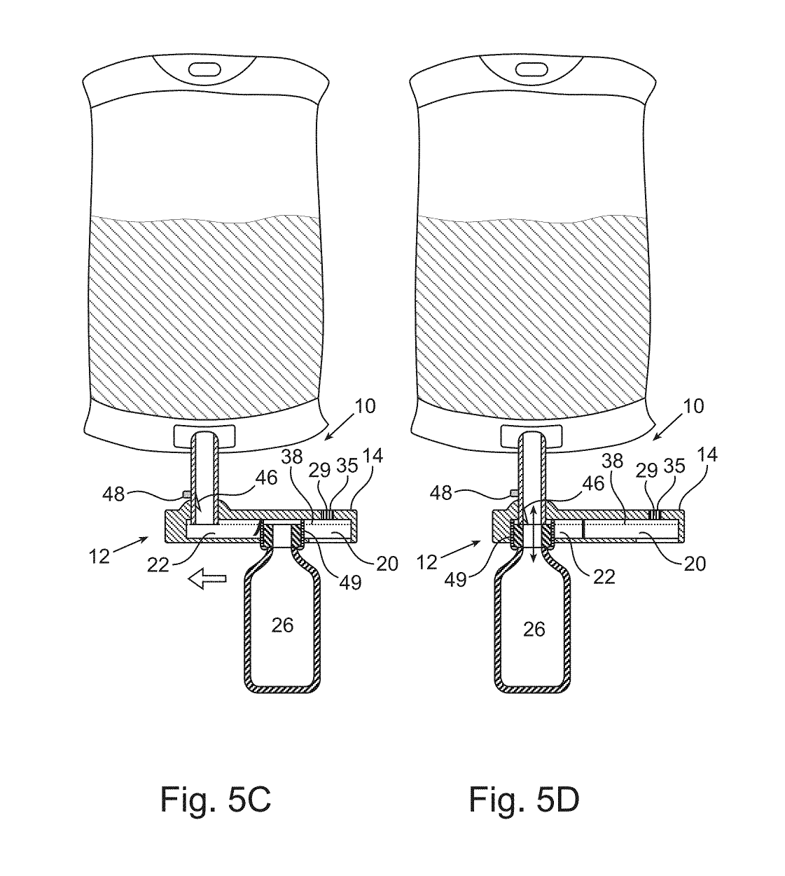

FIGS. 5A-5D shows system 10 which further comprises an engagement mechanism with clamping element 49 configured to provide an engagement between vessel 24 and vessel 26, or between housing 12 and vessel 26. The engagement mechanism in this case is located at first compartment 20. The clamping element 49 is attached/coupled to rail 38 allowing it to move from a first position to a second position along with vessel 26. In one or more embodiments, first compartment 20 may be sealed and/or covered by a seal/cover (not shown) positioned along the bottom wall of the housing 12 where vessel 26 attaches. In one or more embodiments, the seal/cover (not shown) may be frangible, breakable, and/or rupturable. In one or more embodiments, compartment 20 of housing 12 may contain a sterilizing and/or disinfecting substance to aid in the decontamination of surface 30 of vessel 26. Optionally, compartment 22 may container a sterilizing and/or disinfecting substance to maintain compartment 22 in a decontaminated state once vessel 26 is positioned in compartment 22.

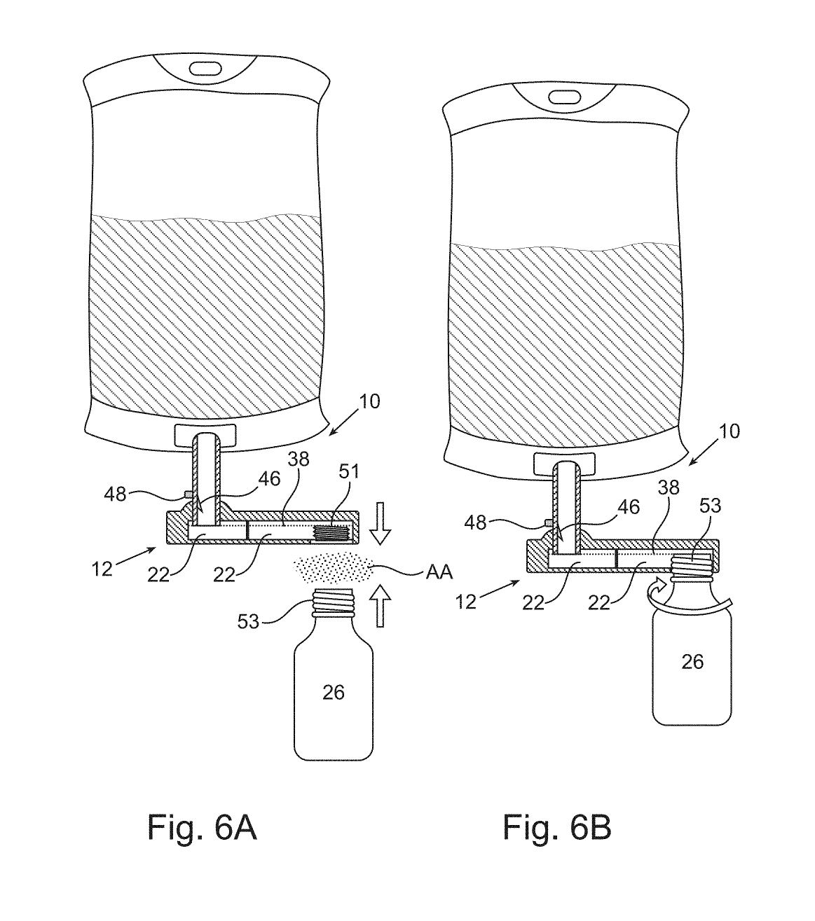

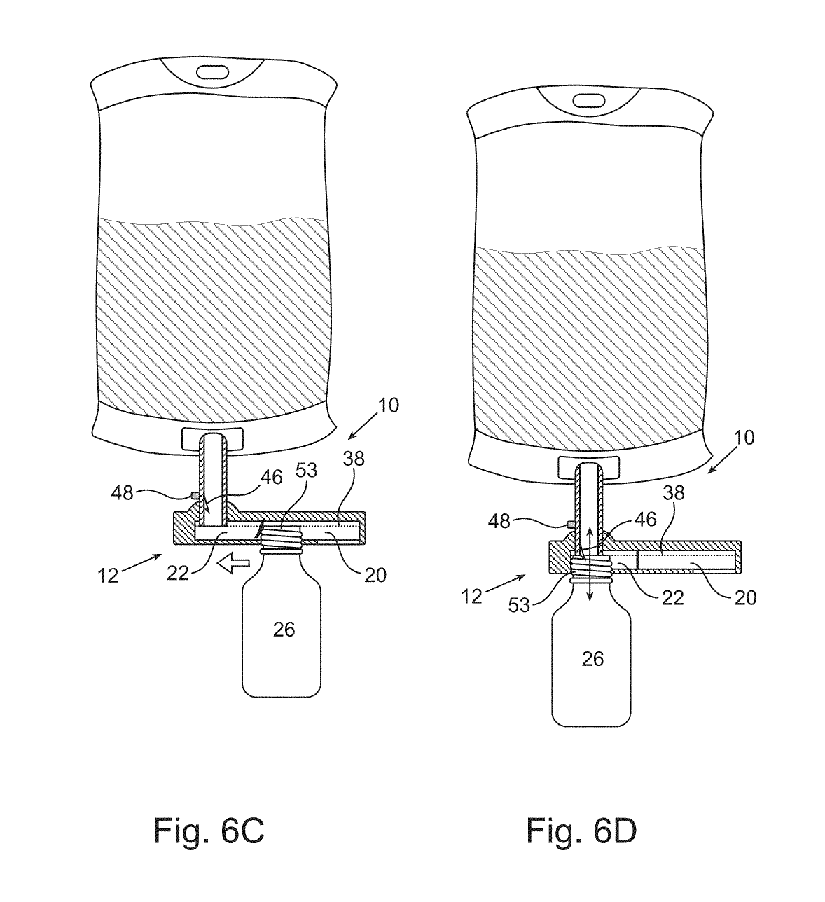

Other engagement mechanisms are herein contemplated. For example, FIGS. 6A-6D demonstrate an engagement mechanism in the form of a thread 51 attached to rail 38 and a complementary thread 53 attached to vial 26.

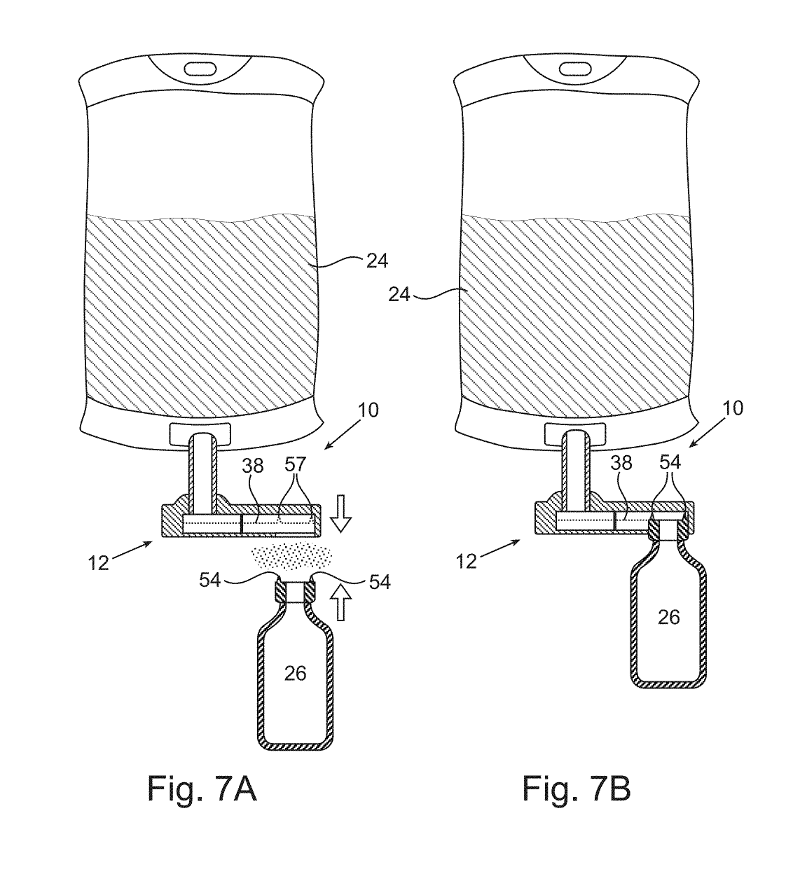

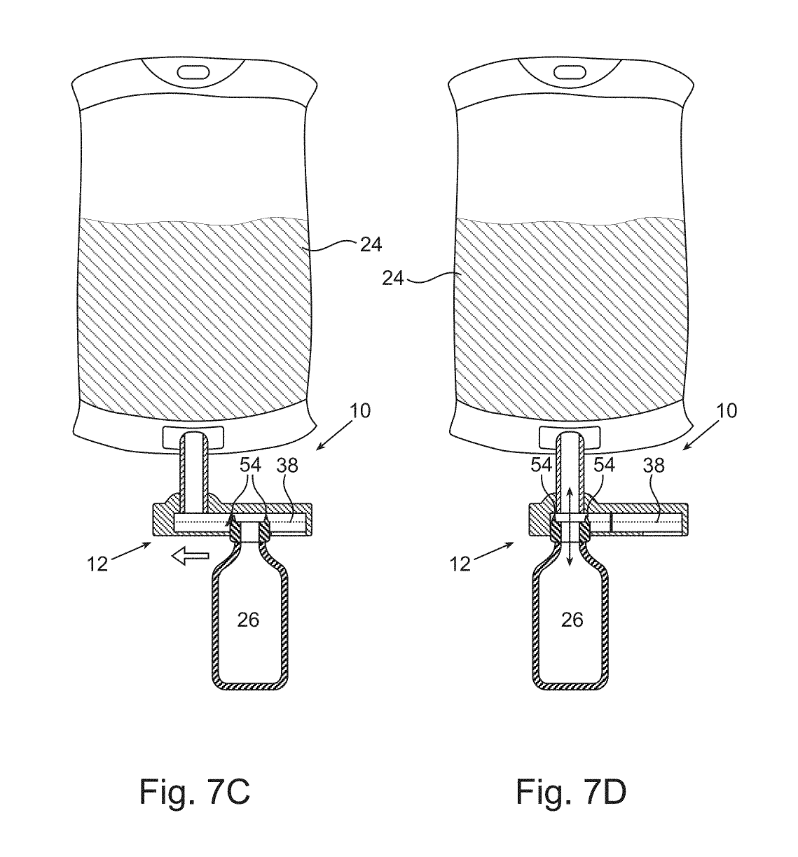

FIGS. 7A-7D demonstrate an engagement mechanism in the form of ratchet teeth 54 disposed on vial 26, which attach complementary ratchet teeth 57 disposed within, on, or between rail 38.

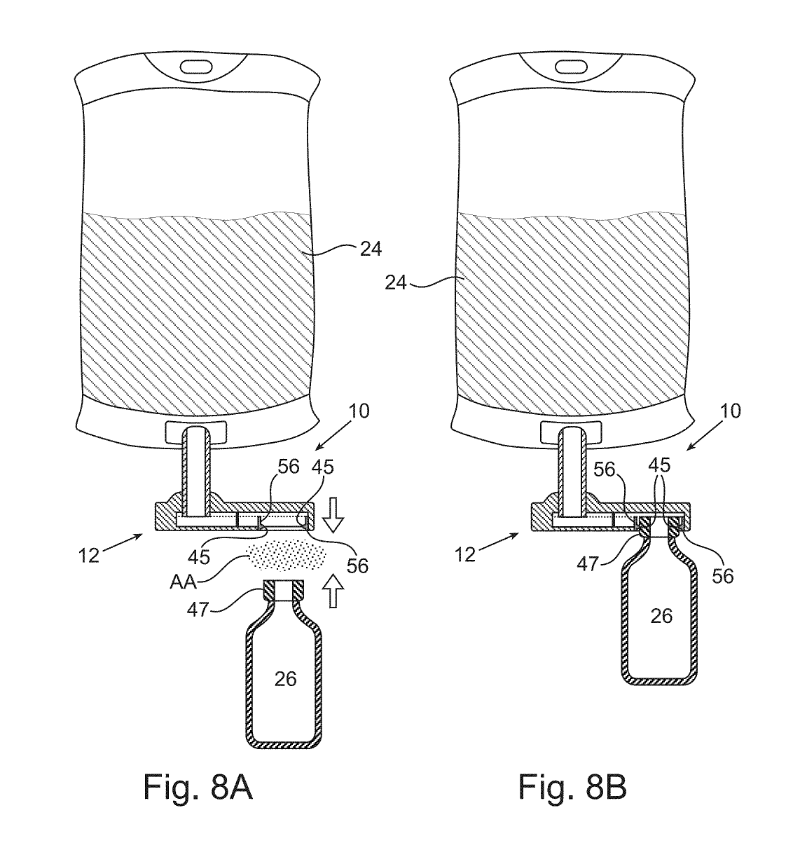

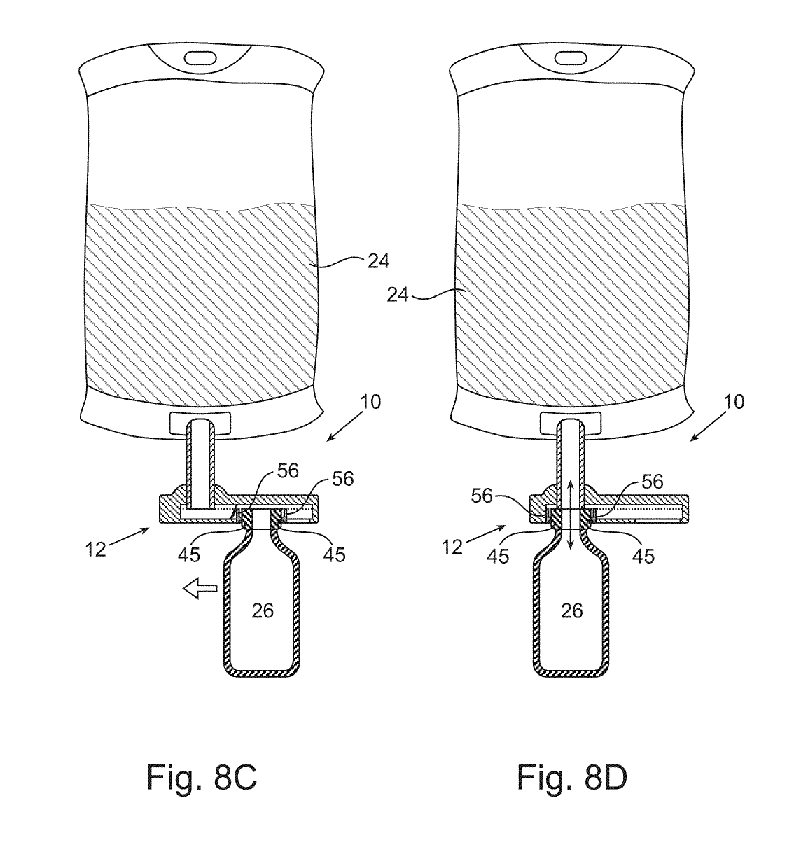

FIGS. 8A-8D demonstrate system 10 which includes circumferential wipers 56 that include protruding rubbers 45. Circumferential wipers 56 provided with protruding rubbers 45 to allow wiping the sides of vessel's port 47 when engaging with housing 12. Optionally, the circumferential wipers 56 with protruding rubbers 45 can be provided also to allow an engagement between vessel 26 and housing 12. Accordingly, rubbers 45 allow wiping off side walls of port 47 under great friction, or under a friction sufficient to decontaminate the sides of port 47 of vessel 26. The rubbers 45 may also be made of a sponge-like material. In one or more embodiments, the rubbers 45 may have a disinfecting and/or sterilizing substance covering at least a portion of the rubbers 45 to aid in the decontamination of side walls of port 47.

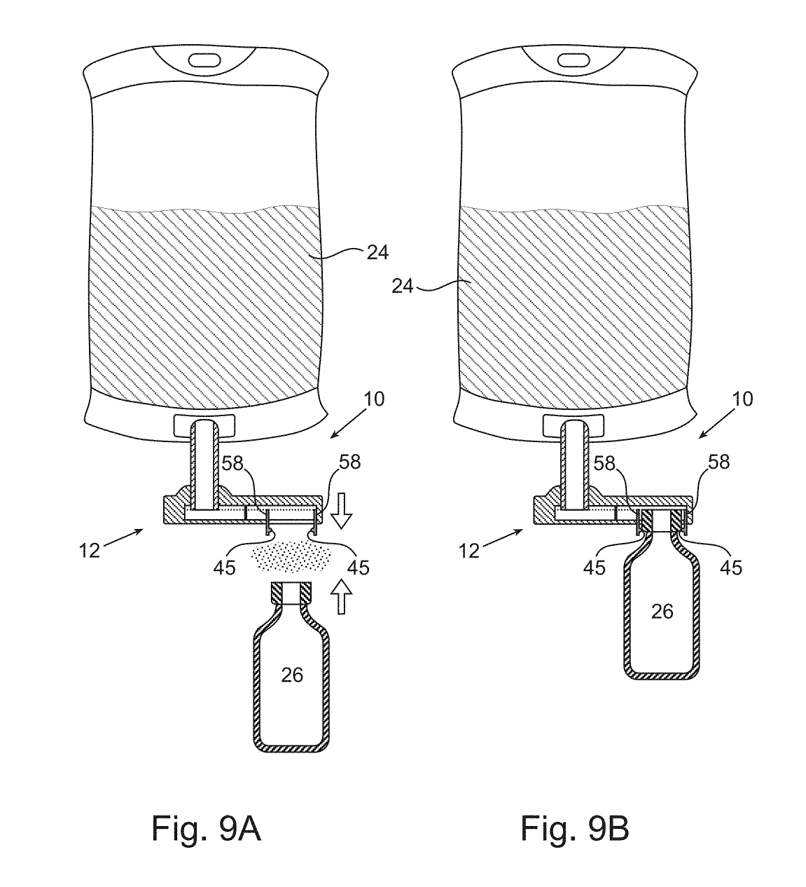

FIGS. 9A-9B demonstrate circumferential wipers 58 that are elongated and that include protruding rubbers 45. The circumferential wipers 58 are in this case elongated extending outwardly from housing 12.

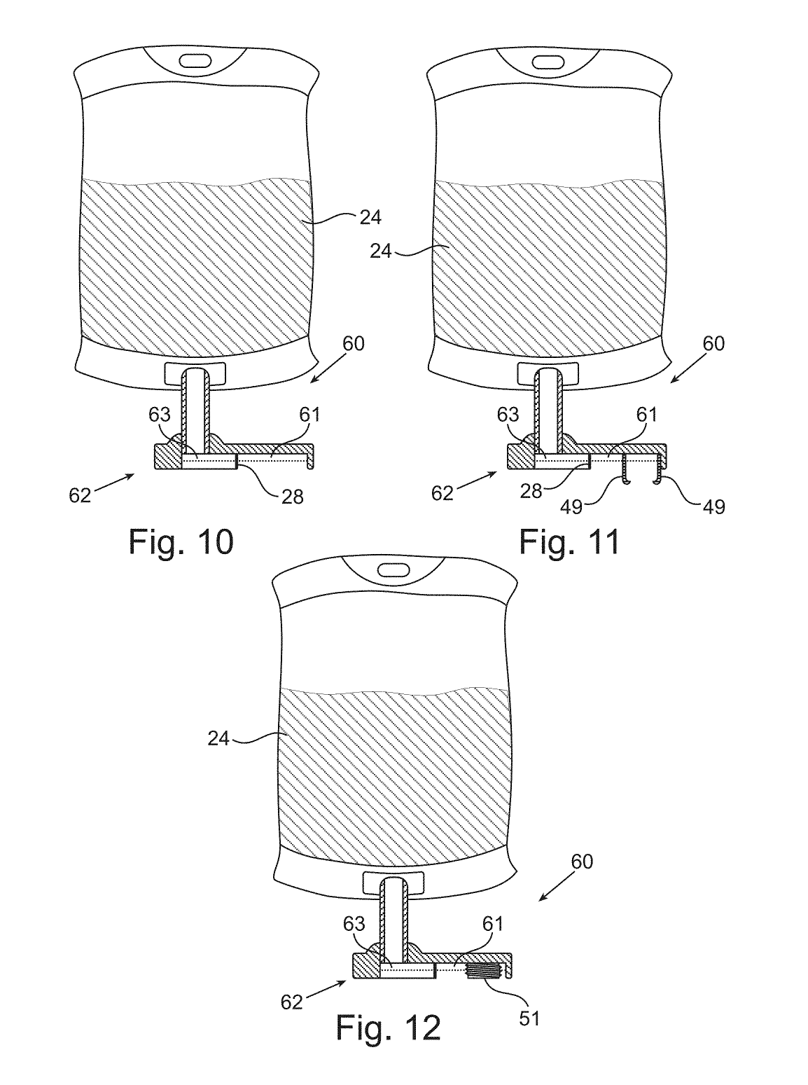

FIG. 10 shows yet a further exemplary system 60 with housing 62 wherein only first compartment 61 is open and second compartment 63 is closed or at least partially closed.

FIG. 11 shows system 60 which alternatively includes an engagement mechanism in the form of clamping element 49, and FIG. 12 shows system 60 with an engagement mechanism having thread 51 configured to attach complementary thread 53 present on a second vessel. Clamping element 49 and thread 51 may be positioned on and/or between the rails (horizontal dotted line within housing 62) of housing 62. Optionally, clamping element 49 and thread 51 may be attached, integrally attached, and/or form a unitary structure with the rail (horizontal dotted line within housing 62). The rail (horizontal dotted line within housing 62) provides for the movement of a vessel attached/engaged to clamping element 49 or thread 51 from an initial position in compartment 61 to a secondary position in compartment 63.

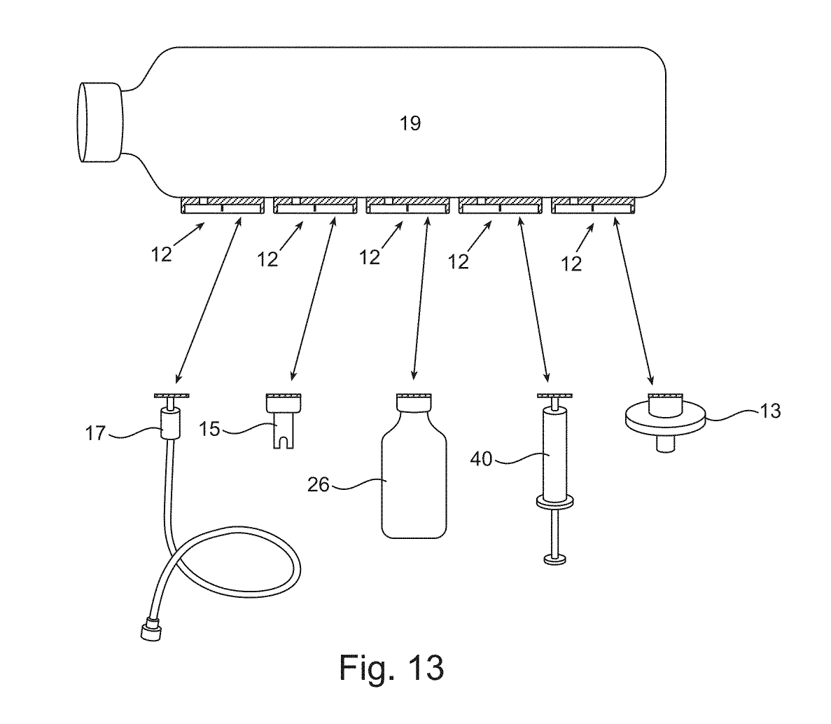

FIG. 13 demonstrates a vessel 19 which is connected to a plurality of housings 12, each of which are configured to connect a medical vessel, such as filter 13, syringe 40, vial 26, connector 15 and infusion line 17. Vessel 19 can therefore connect a plurality of medical vessels in a decontaminated manner. In one or more embodiments, housings 12 may be surface mounted to vessel 19. Optionally, housings 12 may be flush mounted (not shown) to vessel 19. Optionally, housings 12 may abut a wall of vessel 19. In on ore more embodiments, the plurality of housings 12 may engage a plurality of vials 26. In one or more embodiments, the plurality of housing may all engage a plurality of vials 26. In one or more embodiments, the plurality of housings 12 may engage and decontaminate a plurality of vials 26. In one or more embodiments, the plurality of housing 12, may engage and decontaminate a plurality of syringes.

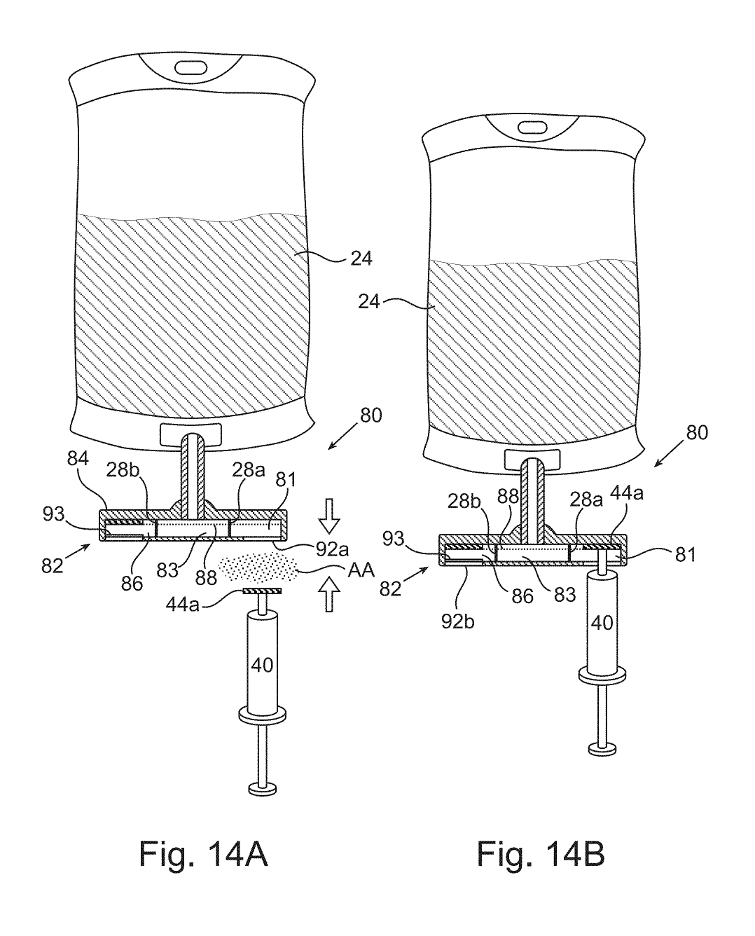

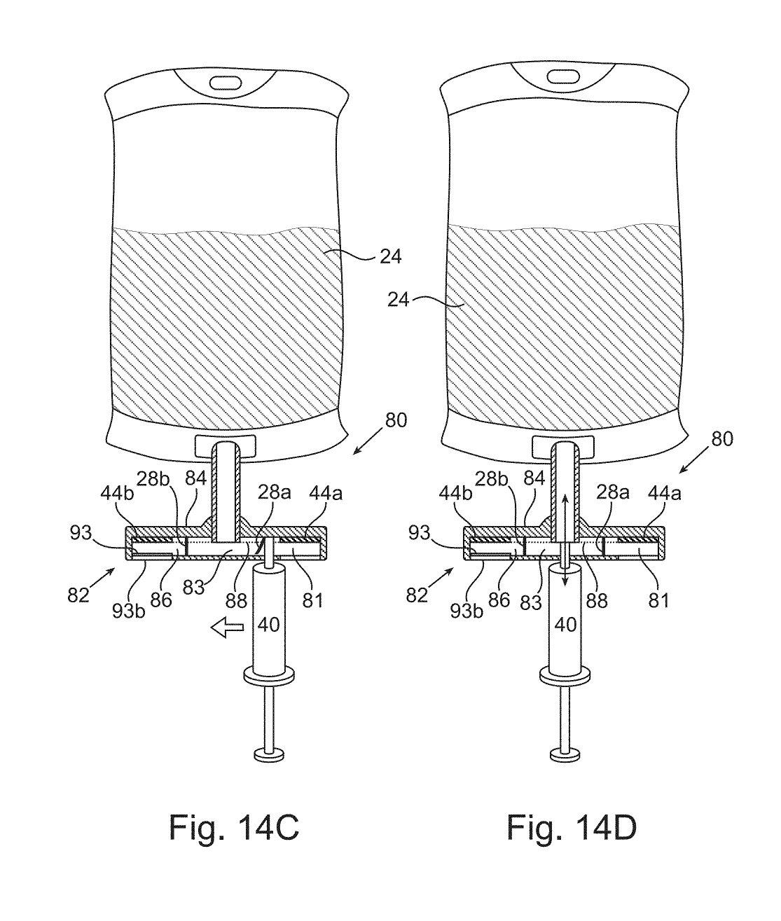

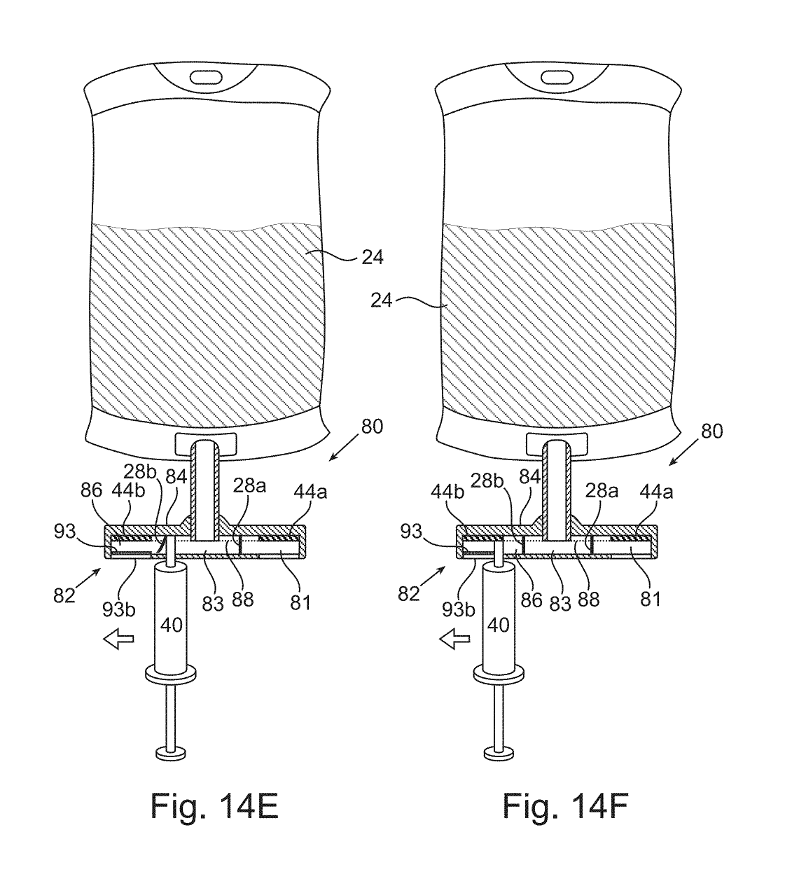

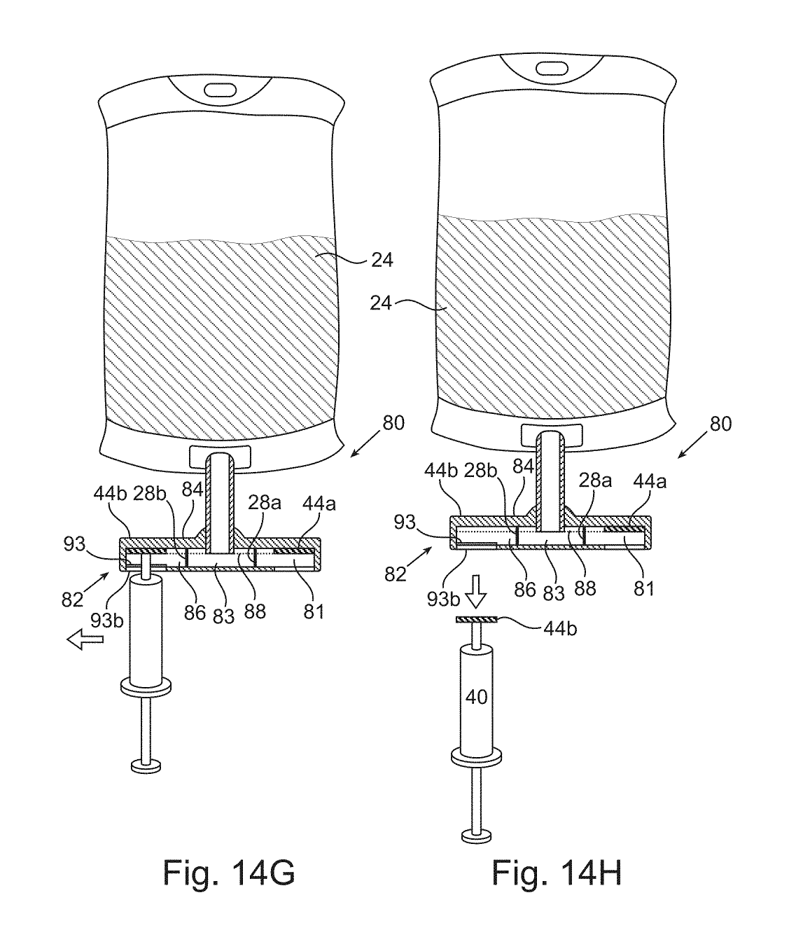

FIGS. 14A-14H demonstrate yet a further exemplary system 80 with a housing 82 which includes three compartments, being first compartment 81, middle second compartment 83, and third compartment 86. First compartment 81 is non-sterile, and second compartment 83 being sterile or essentially sterile and maintained as such due to both wiping members 28a and 28b which hermetically enclose second compartment 83. Optionally, third compartment 86 is further closed by a fragile seal 93 covering bottom opening 92b. The seal 93 can be ruptured when syringe 40 moves to third compartment 86 or when syringe 40 detaches from third compartment 86. First wiping member 28a is disposed between first and second compartments 81 and 83, respectively and second wiping member 28b is disposed between second and third compartments 83 and 86, respectively. FIG. 14A shows syringe 40 with displaceable member 44a which functions as a seal or a cap optionally, being integrally connected to syringe 40. Optionally, displaceable member 44a may be position on a rail mechanism (not shown) of syringe 40 or between the rails of a rail mechanism (not shown) of syringe 40, wherein the rail mechanism in configured to provide for the displacement of displaceable member 44a. Optionally, displaceable member 44a may be positioned on syringe 40 and held in place on syringe 40 by a hinge mechanism (not shown), wherein upon movement of syringe 40 from compartment 81 to compartment 83 or from compartment 83 to compartment 86 displaceable member 44a displaces from aperture (not shown) of syringe 40 via the hinge mechanism.

In FIG. 14B, syringe 40 enters housing 82 via first bottom opening 92a and engages with rail 88 and/or the inner surface of top wall 84. At FIG. 14C, syringe 40 is shown when moving along the housing 82 and when displaceable member 44a remains connected to the inner surface of top wall 84. The surface of syringe 40 is then wiped off by wiping member 28a. An alignment with vessel 24 and fluid passageway is then established (shown is FIG. 14D). Following fluid transfer, between the vessels 24 and 40, syringe 40 continues to travel along housing 82 via rail 88 and moves from second compartment 83 to third compartment 86. Syringe 40 then travels towards third compartment 86 and passes through another second wiping member 28b which wipes off any fluid residuals present on surface of syringe 40 (shown in FIG. 14E). FIGS. 14F-14G demonstrate syringe 40 being resealed by resealing mechanism that includes second displaceable member 44b. Resealed syringe 40 with second displaceable member 44b may then exit housing 82 via second bottom opening 93b as shown in FIG. 14H.

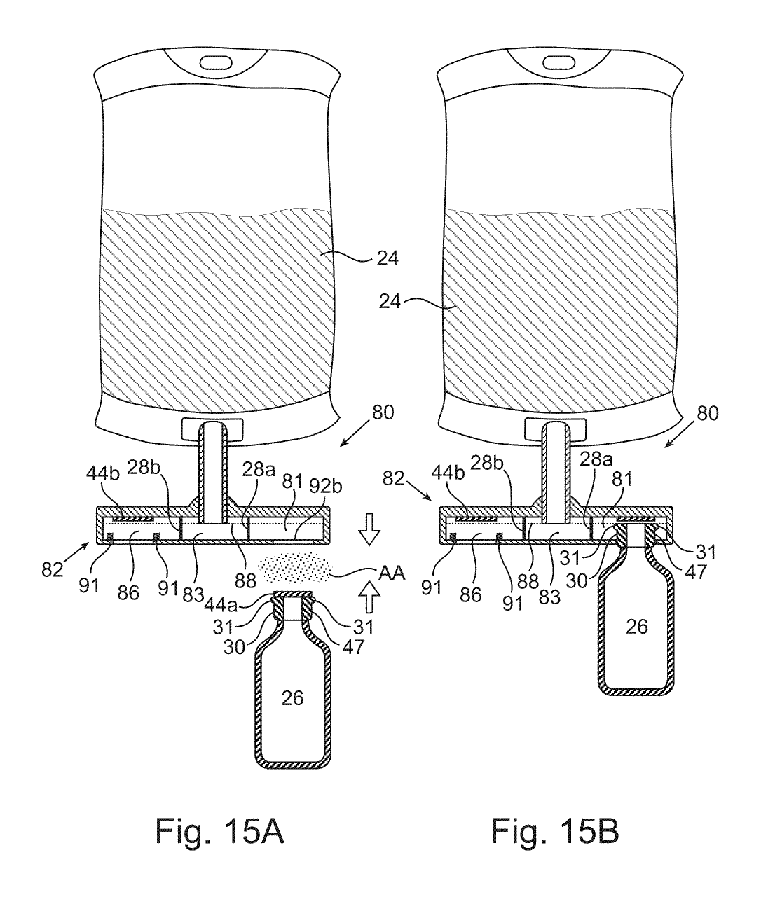

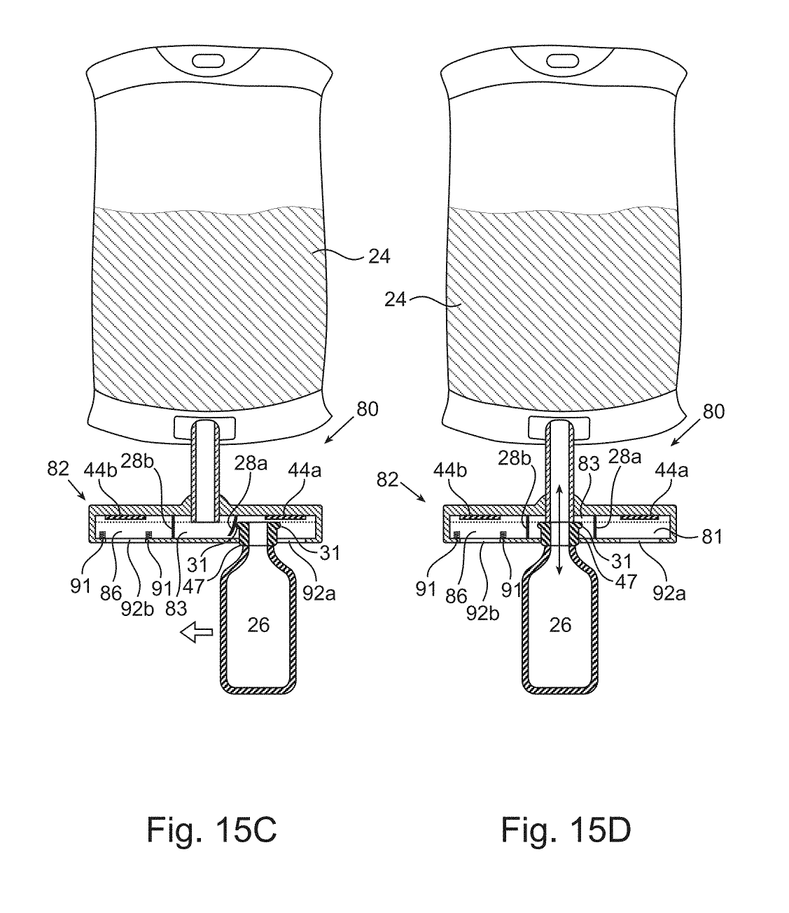

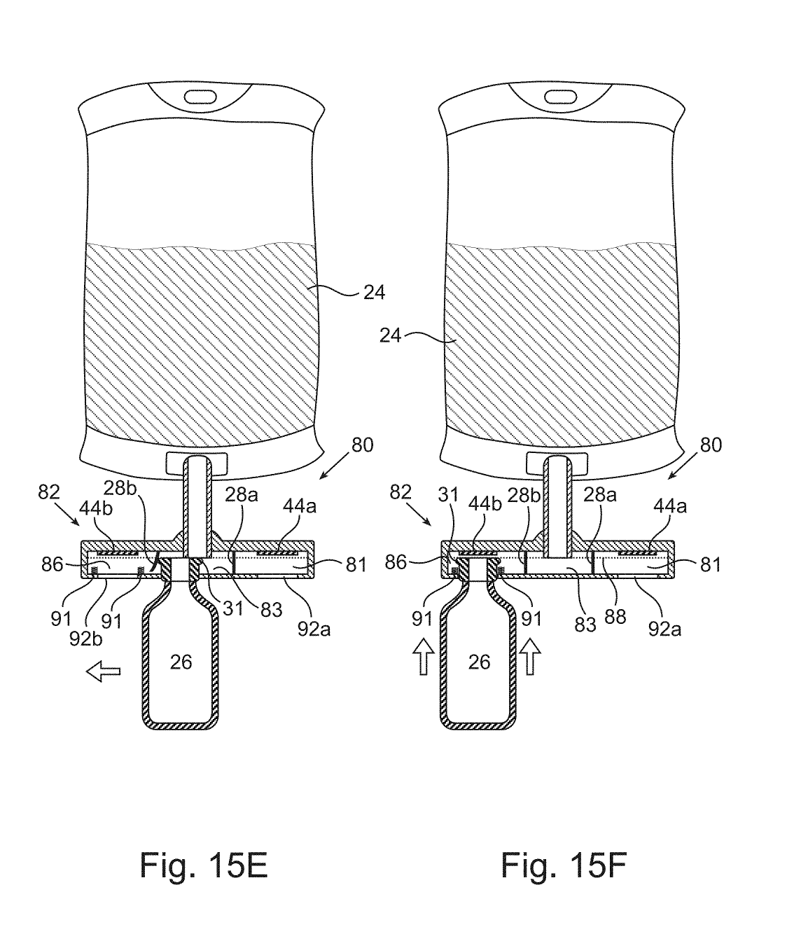

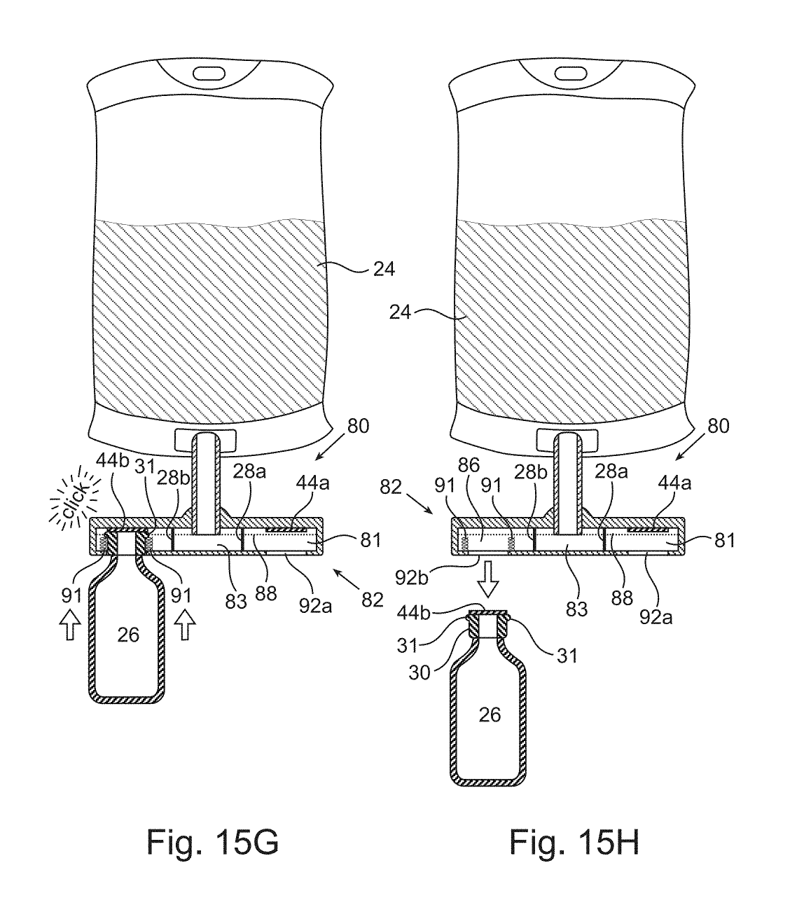

FIGS. 15A-15H demonstrate system 80 in which resealing mechanism further includes top springs 91 disposed at opposing ends of second bottom opening 93b. At FIGS. 15B-15C, vial 26 which here includes protruding ends 31 on the circumference of surface 30 engages with housing 82 at first compartment 81 and displaceable member 44a externally displaces from port 47 at about the time vessel 26 moves from first position at compartment 81 to second position at compartment 83. A fluid passageway between vessel 24 and vessel 26 is then formed (FIG. 15D) via a piercing member disposed within system 80 (not shown) or via breaking of a frangible seal (not shown) of vessel 24 and/or 26. FIG.15E shows movement of vessel 26 towards compartment 86. Surface 30 (as shown in FIG. 5A) is wiped off by wiping member 28b. FIG. 15F demonstrates vessel 26 situated at third compartment 86. FIG. 15G demonstrates springs 91 which are released, lifting vessel 26 via protruding ends 31 upwardly towards displaceable member 44b to engage with displaceable member 44b. Various types and locations of springs 91 are contemplated. Further contemplated herein are springs 91 which may alternatively or additionally expand to the sides of third compartment 86. Optionally, springs 91 may be a flexible or elastomeric material that allows movement of vial 26 port within third compartment 86 to facilitate engagement between displaceable member 44b and vessel 26. Optionally, springs 91 may allow the movement of a surface of vessel, or vessel 26, horizontally and/or side-to-side within compartment 86 of housing 82. FIG. 15H demonstrates exit of vessel 26 from housing 82 following resealing of vessel 26 with displaceable member 44b. Resealing of vessel 26 by displaceable member 44b occurs inside third compartment 86 which is sterile or contaminant-free. Displaceable member 44b may be attached to an inner wall of compartment 86 of housing 82 via a frangible and/or breakable seal. Upon engagement of vessel 26 to displaceable member 44b, the force applied by a user's hand to detach vessel 26 from housing 82 may break the seal between displaceable member 44b and a wall of compartment 86 of housing 82. At about the time, during, and/or upon engagement of displaceable member 44b to vessel 26 and/or upon detachment of vessel 26 from housing 82 a click sound, or other sound may be produced by the housing 82 of system 80 to alert the user that vessel 26 has been resealed and/or that vessel 26 has detached from housing 82.

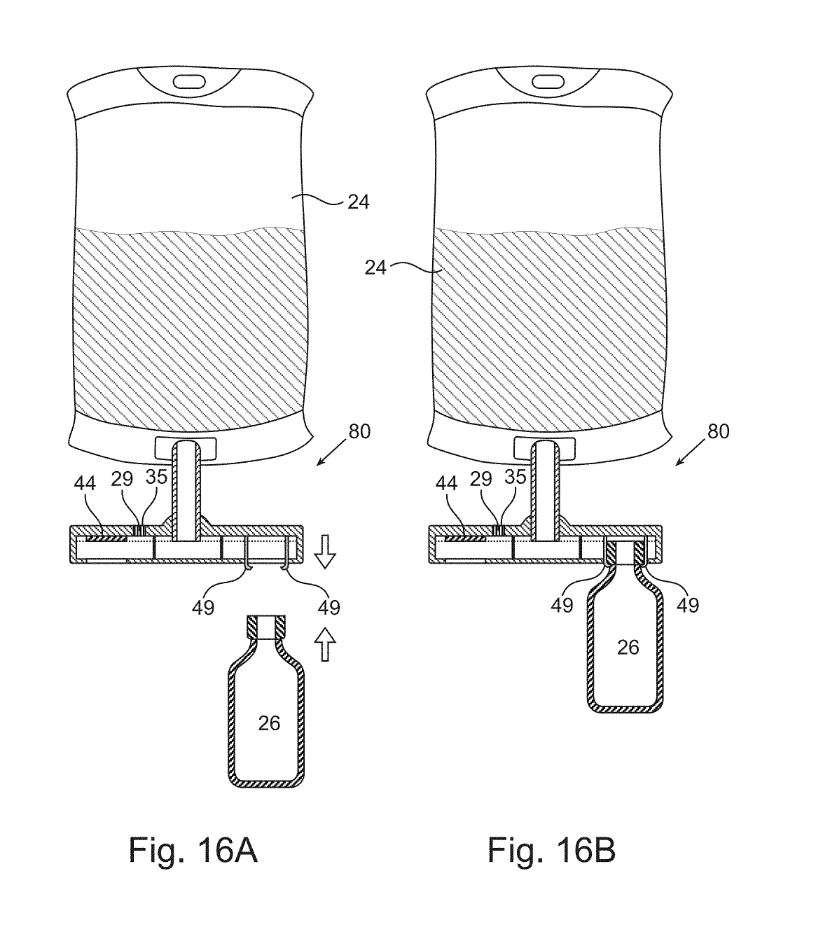

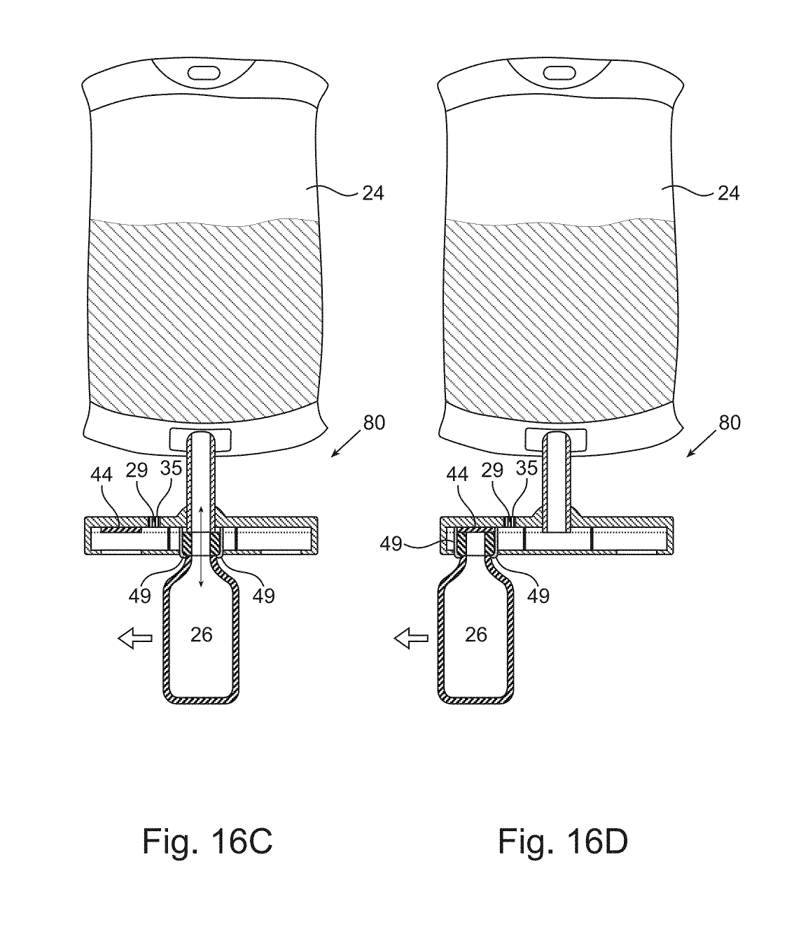

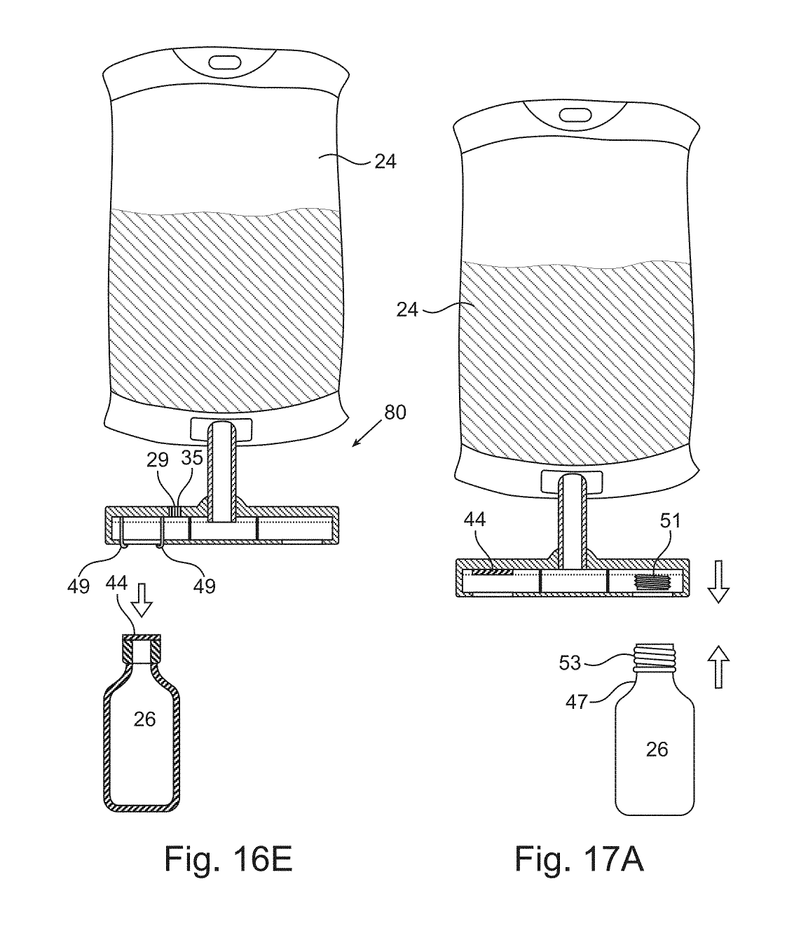

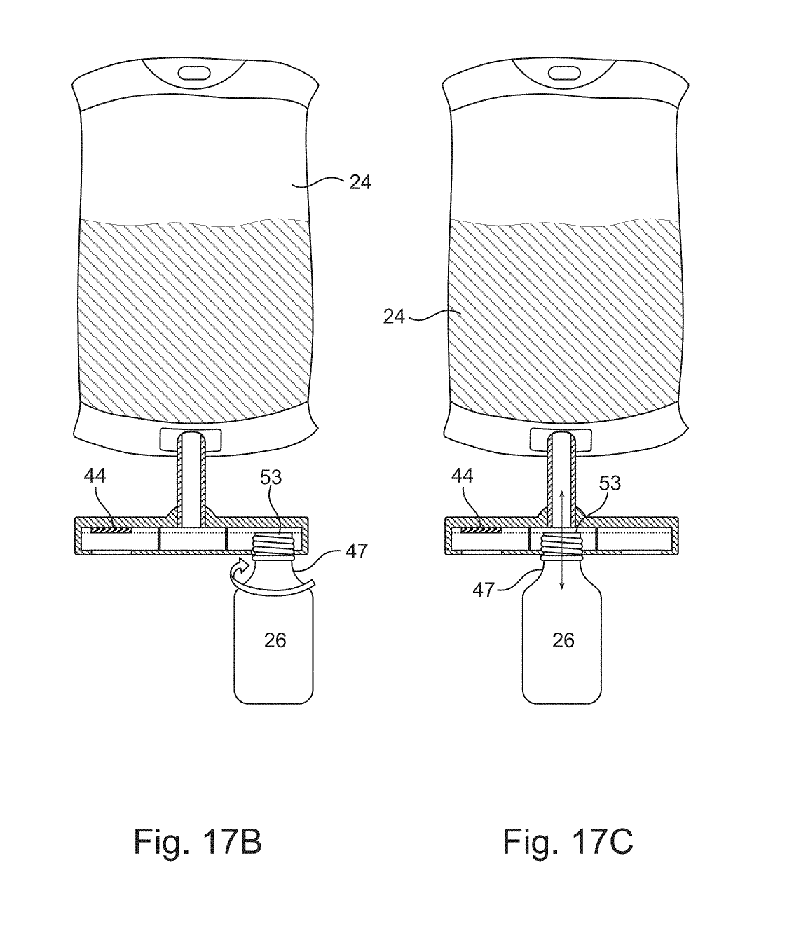

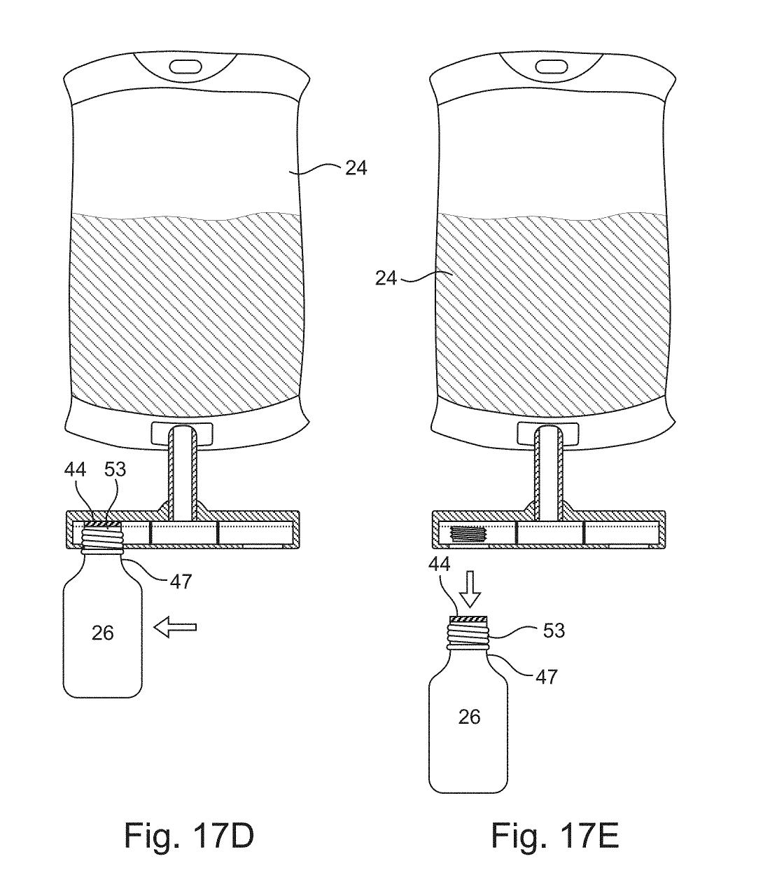

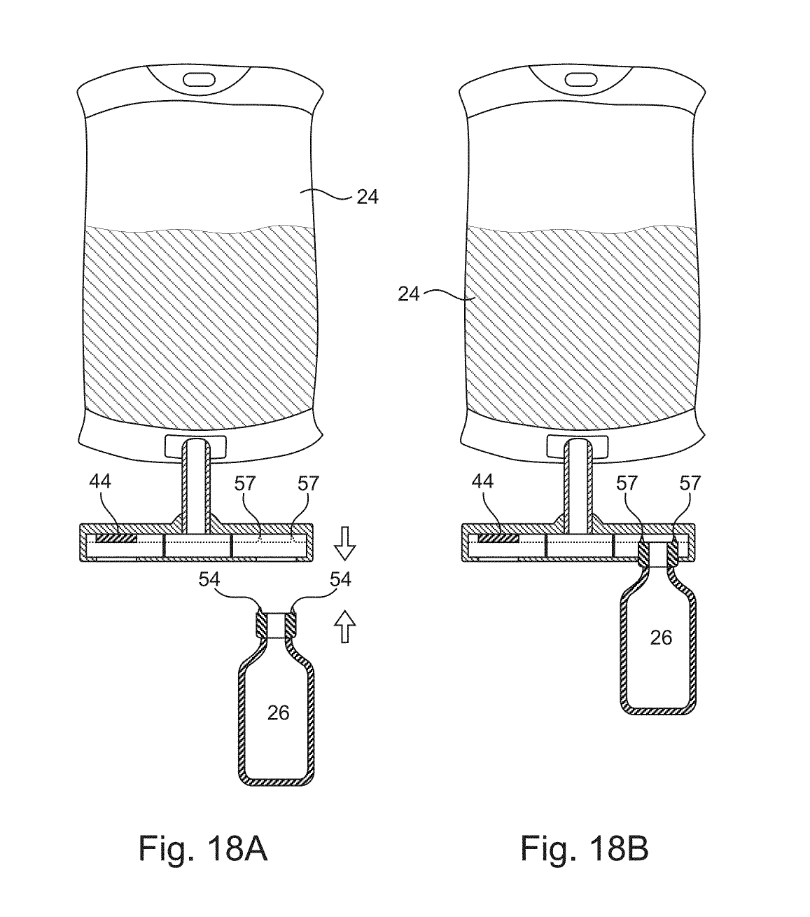

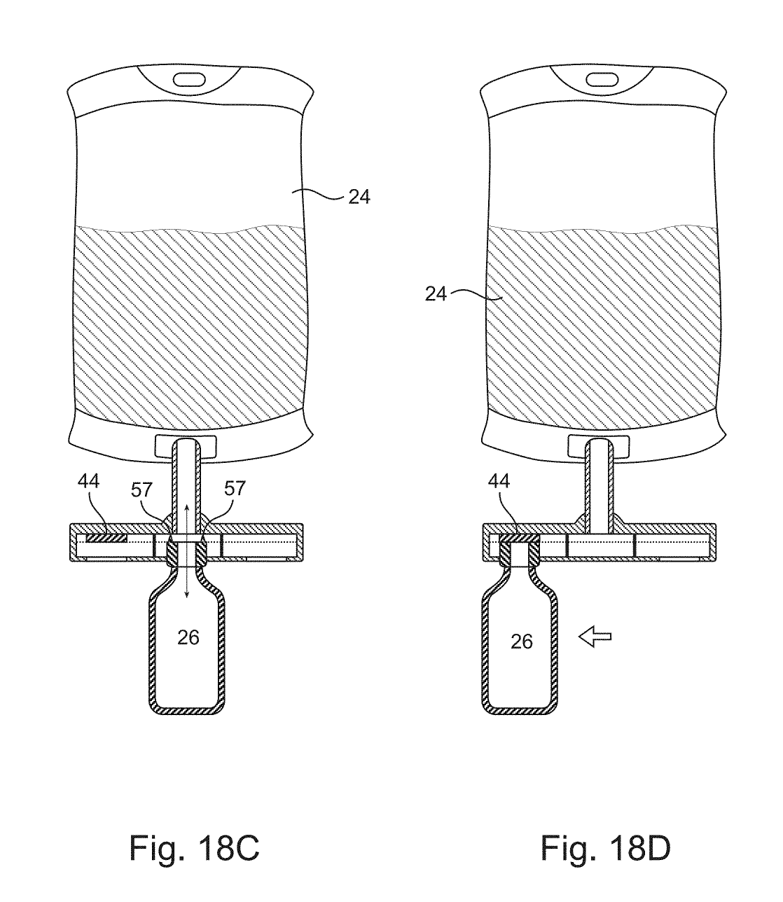

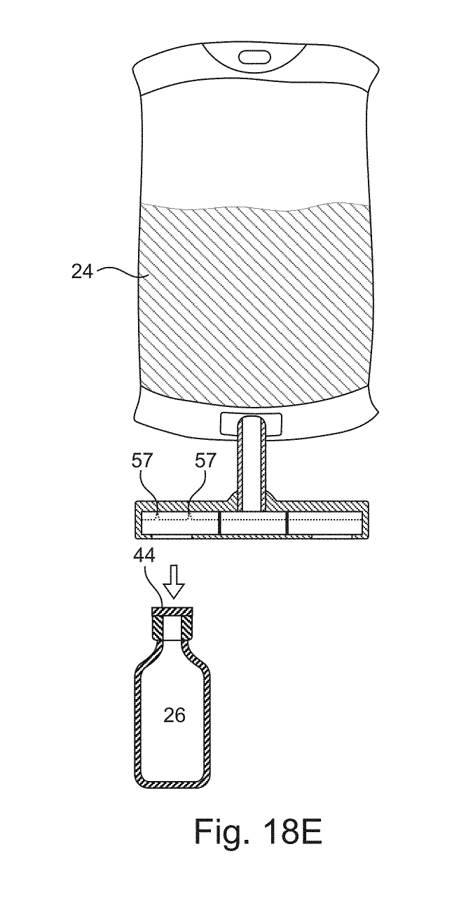

FIGS. 16-18 demonstrate system 80 which may further include an engagement mechanism. As discussed above, various engagement mechanisms are herein contemplated. For example, clamping elements 49 (FIGS. 16A-16E), thread 51 configured to engage complementary thread 53 disposed on the circumference of port 47 of vessel 26 (FIGS. 17A-17E), or ratchet teeth 54 which can attach complementary ratchet teeth 57 (FIGS. 18A-18E).

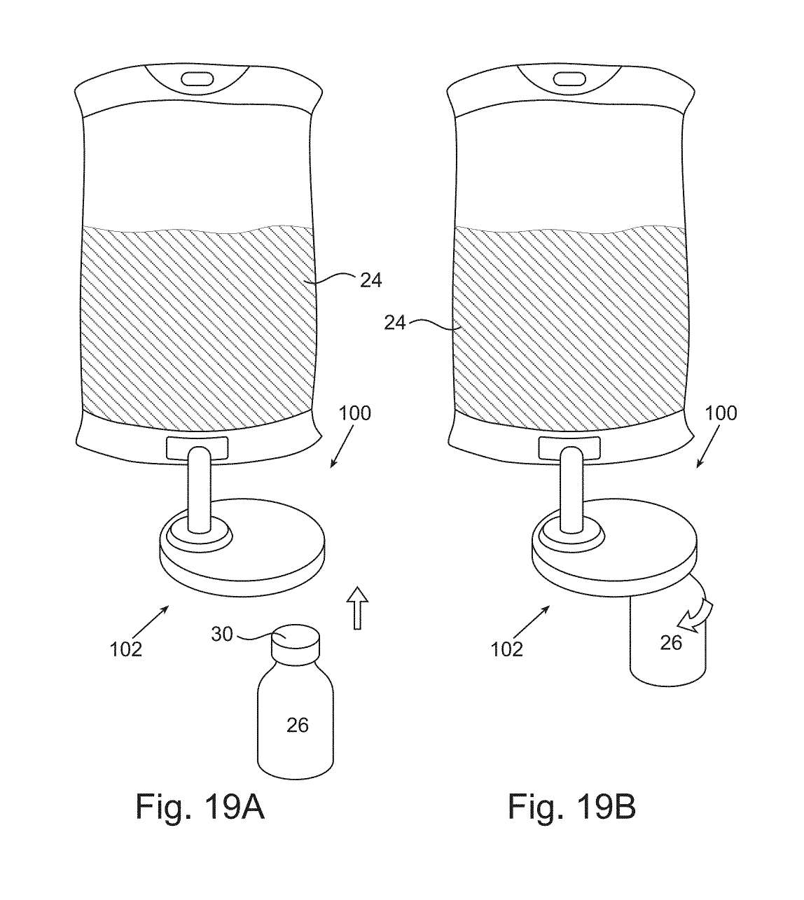

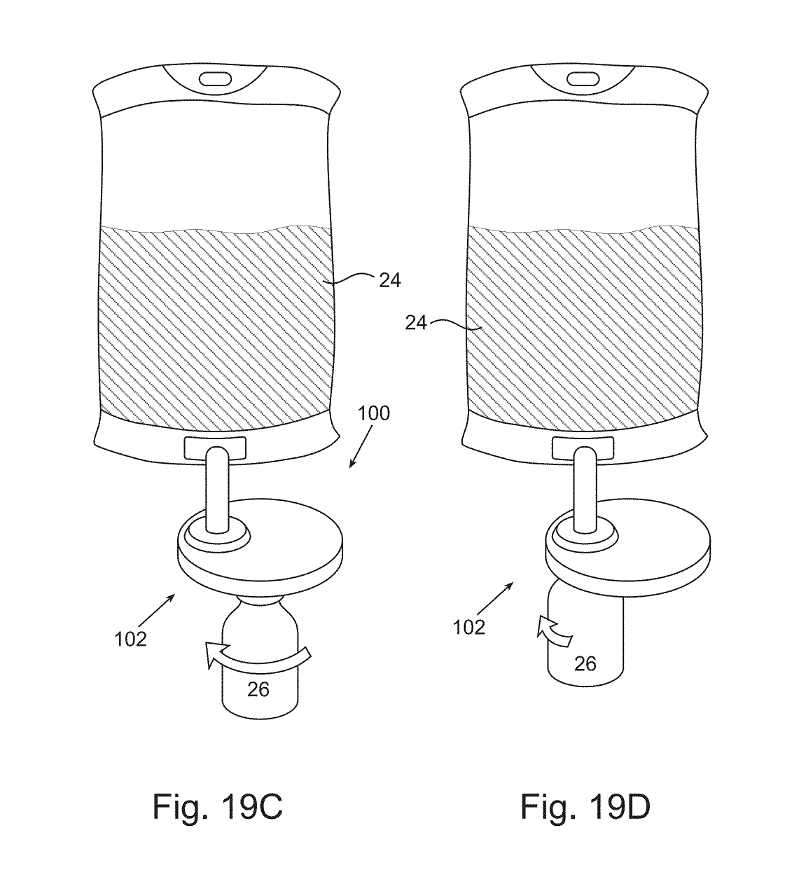

FIGS. 19A-19D are top perspective views demonstrating a further exemplary system 100 which includes housing 102 within which a wiping member is disposed. Vessel 26, following engagement with housing 102, rotates within the housing 102 and while doing so passes through a wiping member that decontaminates the surface 30 of vessel 26. In an embodiment of the invention, the vessel rotates up to about 180°, or about 180°, or above about 60° within housing 102. The vessel 26 may also rotate above about 180° along housing 102. In one or more embodiments, the vessel may rotate less than 360 degrees.

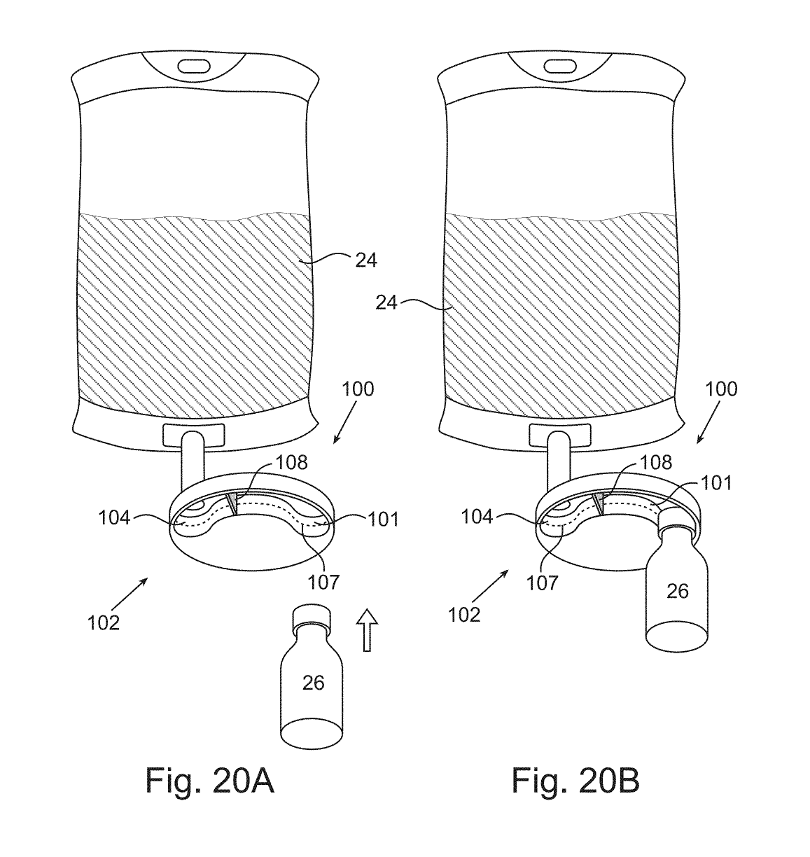

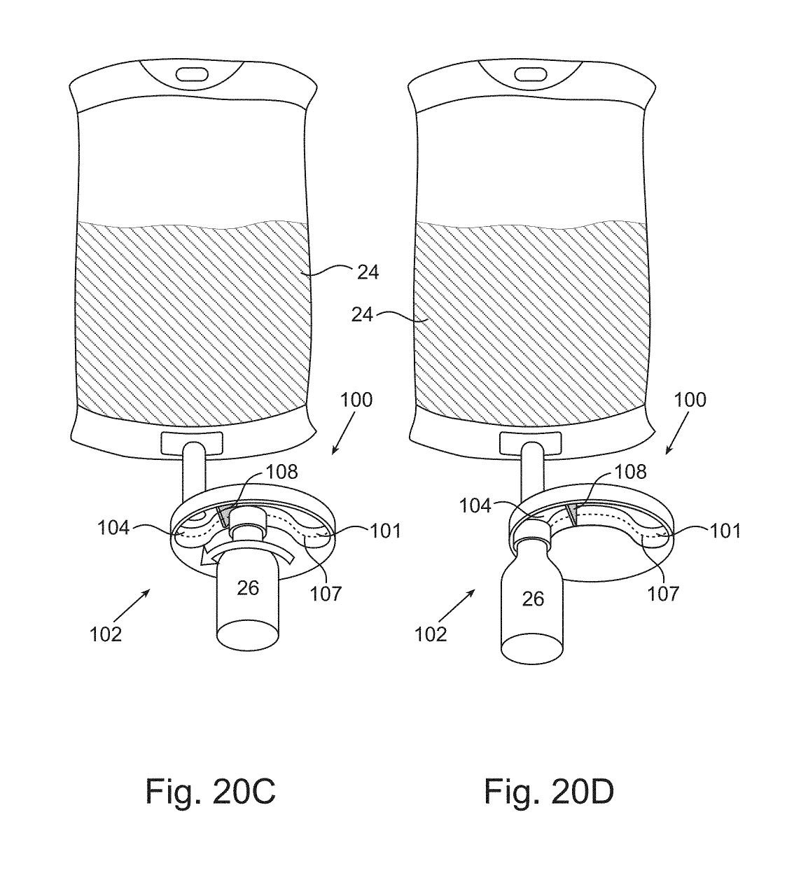

FIGS. 20A-20D are bottom perspective views of system 100. Vessel 26 is configured to attach first compartment 101 and slide along circumferential rail 107 to thereby pass through wiping member 108 and move to second compartment 104 to thereby engage and/or align and/or establish a fluid communication with vessel 24. The vessel 26 thus rotates within housing 102. In one or more embodiments a surface of vessel 26 and/or the port of vessel 26 rotates within housing 102.

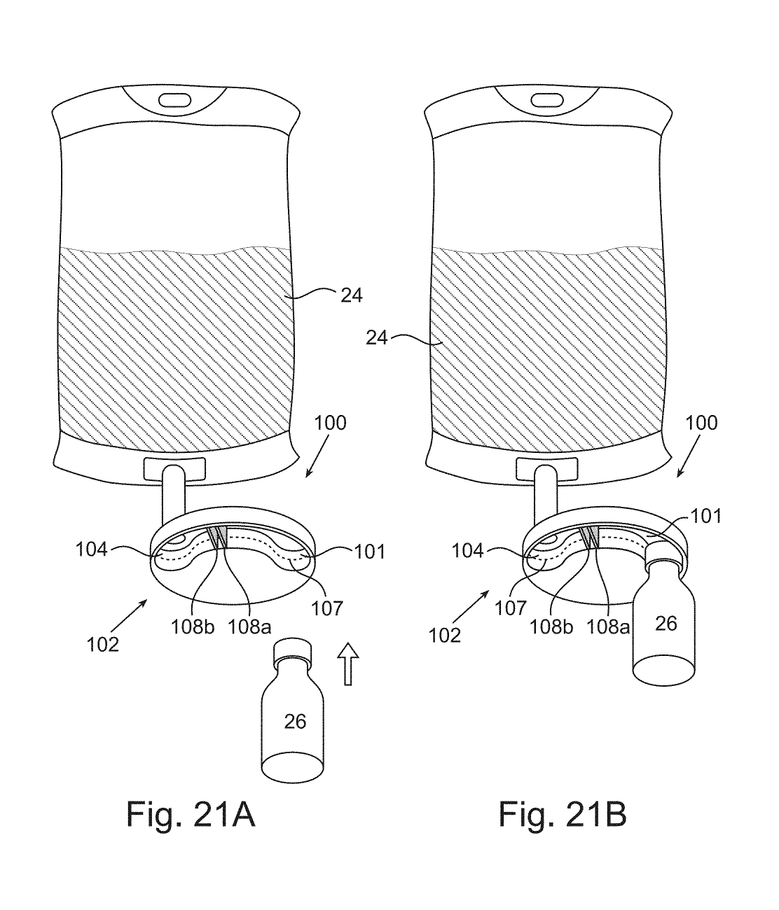

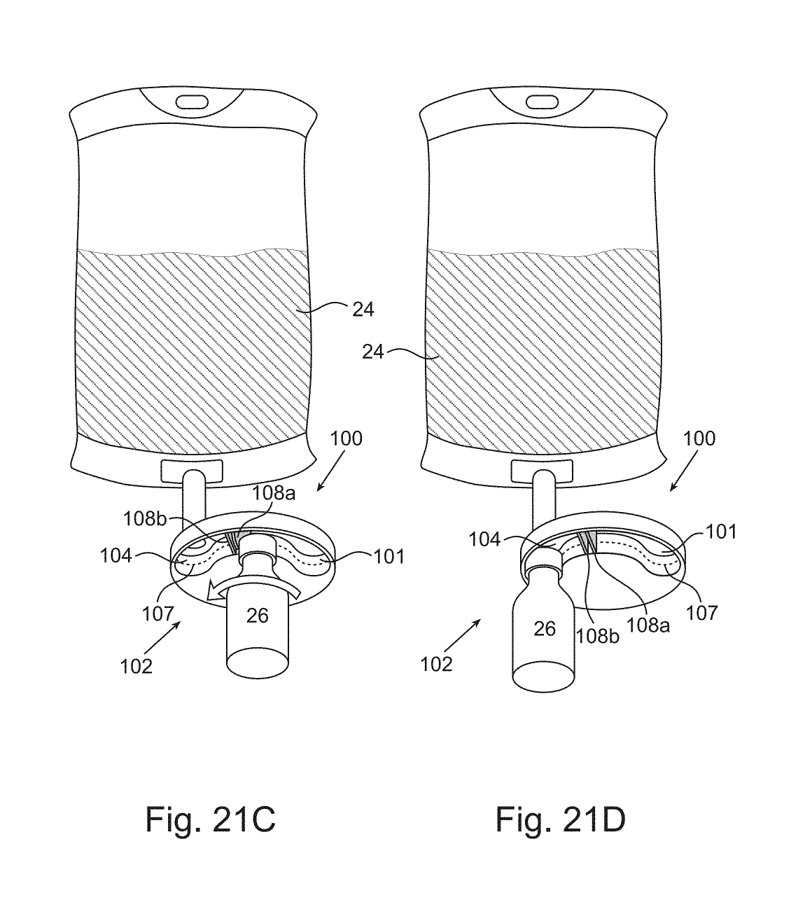

FIGS. 21A-21D illustrate system 100 which may optionally include a double wiper with a first wiping member 108a and a second wiping member 108b. Vessel 26 engages with housing 102 at first compartment 101 and slides along circumferential rail 107. The vessel 26 is firstly wiped off by wiping member 108a and thereafter wiped off by wiping member 108b. In an embodiment of the invention, wiping member 108b is maintained intact, straight and in place when wiping member 108a wipes off vessel 26, thereby preserving second compartment 104 sterile.

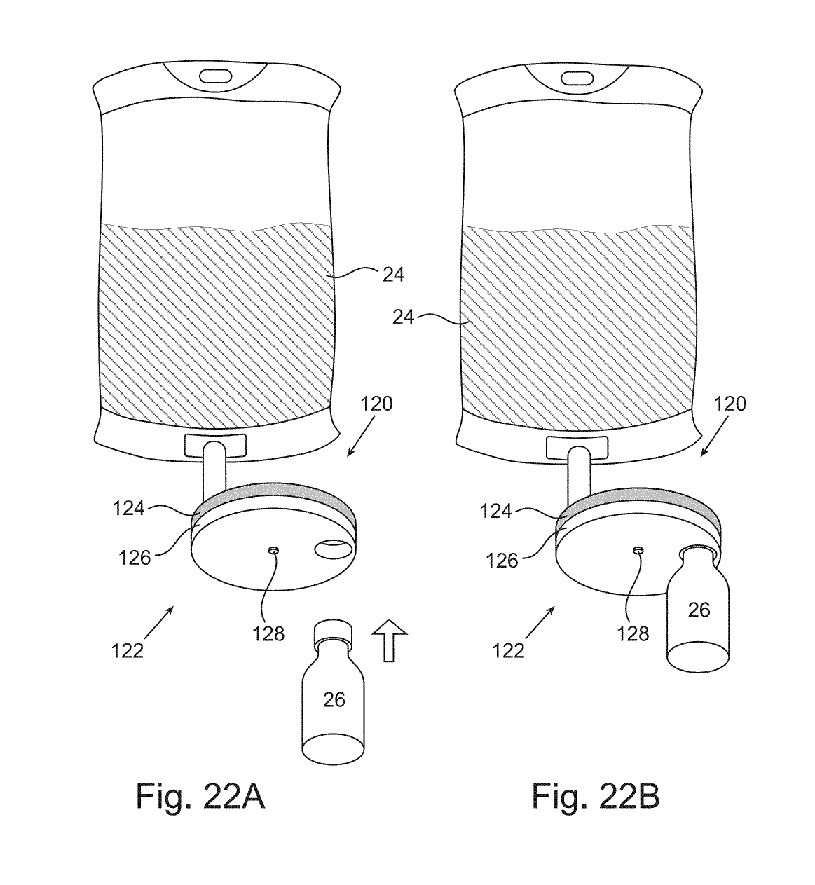

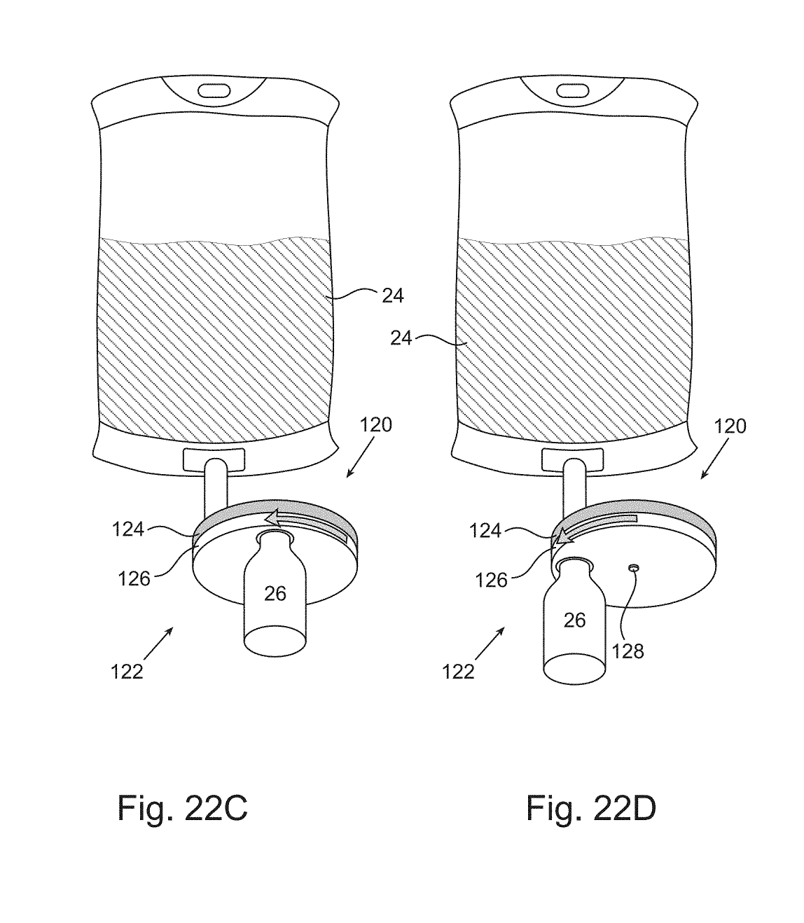

FIGS. 22A-22D demonstrate system 120 which includes a housing 122 with a sliding mechanism in the form of first disc 124 and a second disc 126. First disc 124 and second disc 126 are disposed one above the other and are rotatable with respect to each other via a hinge mechanism 128. One or more wiping members is disposed between the discs 124 and 126, such that upon engagement of vessel 26 with second disc 126 and rotation within housing 122, surface 30 of vessel 26 can be wiped off by the wiping member. In this system 120, the sliding mechanism or hinge mechanism includes at least one disc. Optionally, in this system 120, the sliding mechanism or hinge mechanism includes two discs. Optionally, system 120 may include a three-disc system (not shown), wherein the third disc (not shown) is positioned above first disc 124 and configured to rotate in relation to first disc 124 via hinge mechanism 128 thus providing for a surface of vessel 24 to be wiped off by a wiping member disposed between the third disc (not shown) and the second disc 124.

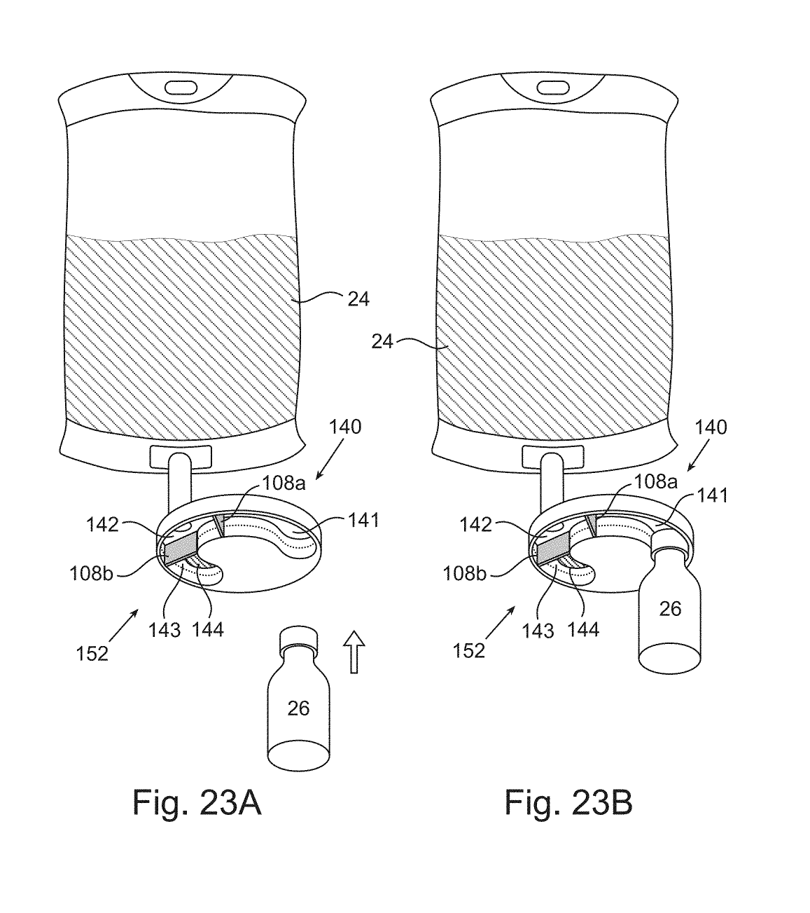

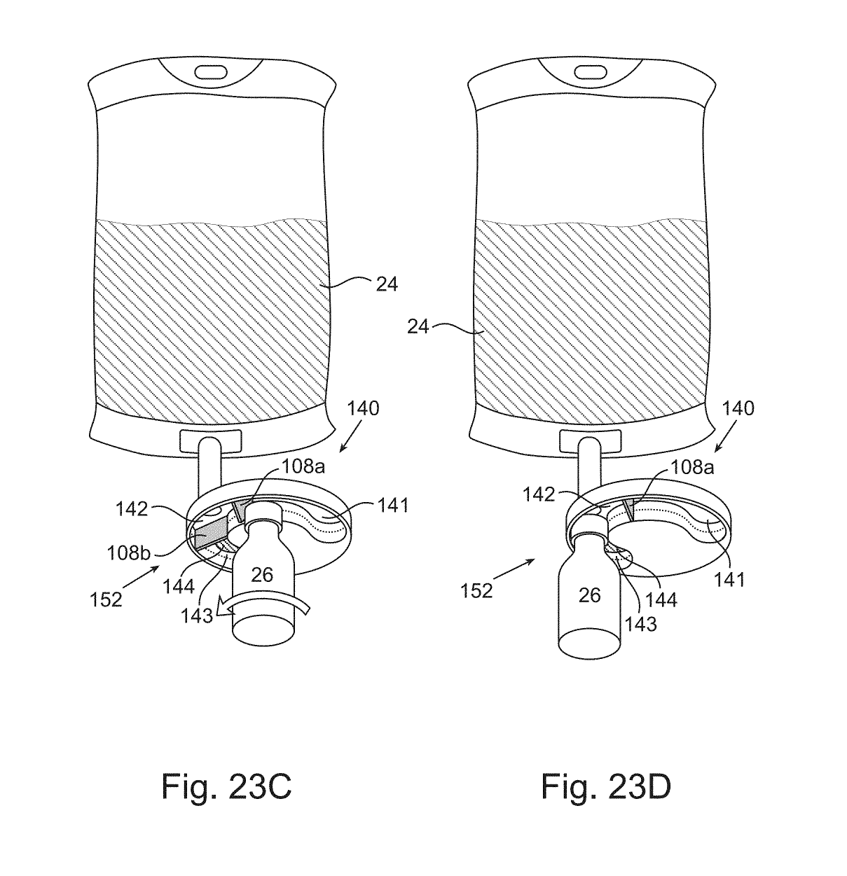

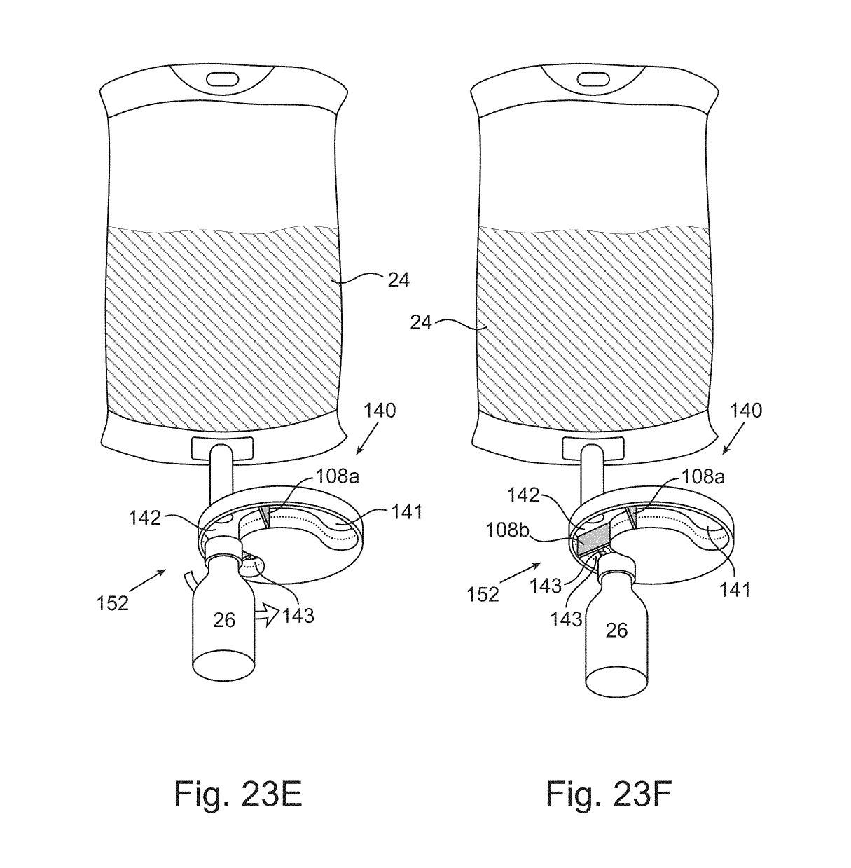

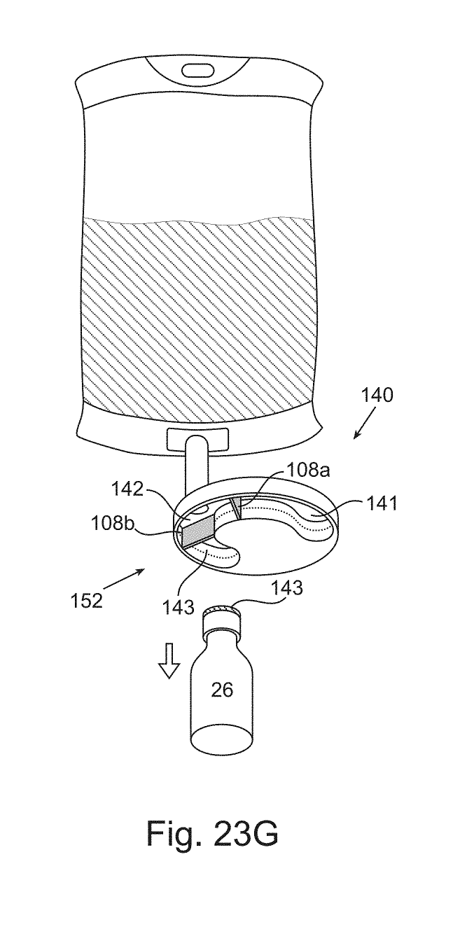

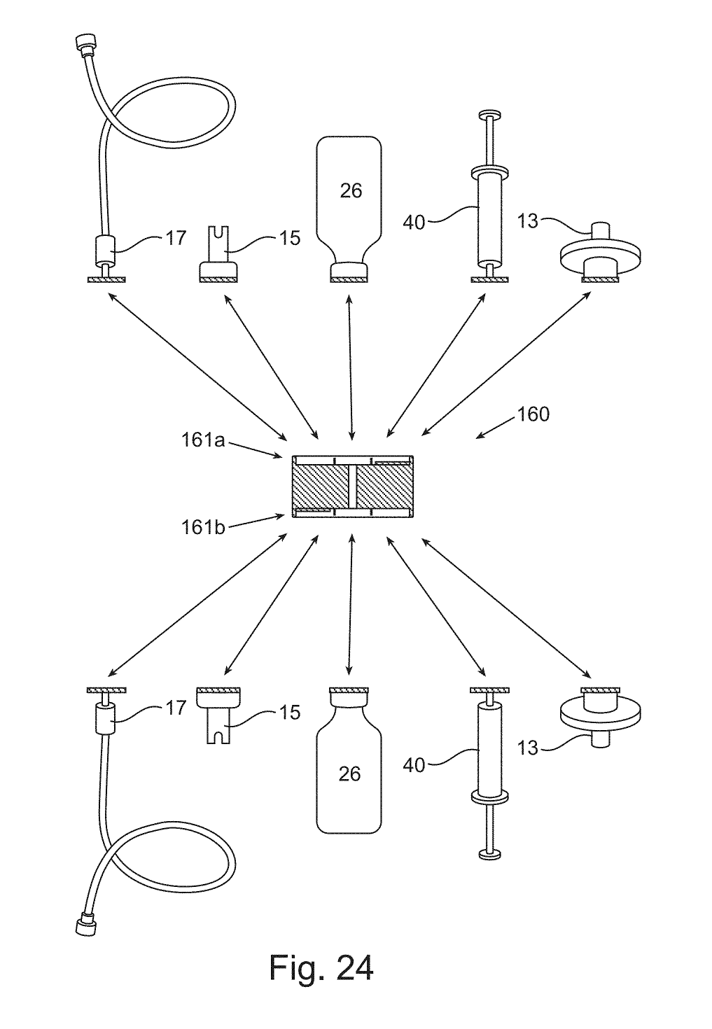

FIGS. 23A-23G demonstrates yet a further exemplary system 140 which is similar to system 100 but includes a housing 152 with three compartments and a resealing mechanism. First compartment 141 is configured for allowing an engagement with vessel 26. First compartment 141 is therefore non-sterile. The system 140 further includes a second compartment 142 and a third compartment 143. First wiping member 108a is disposed between first compartment 141 and second compartment 142. Second wiping member 108b is disposed between second compartment 142 and third compartment 143. The third compartment 143 includes a resealing mechanism having a displaceable member 144, such that the vessel 26 may be resealed when detached from housing 152.

FIG. 24 demonstrates a connector 160 which includes two housing elements, being top housing 161a and bottom housing 161b disposed one above the other and integrally connected. Housing 161a is arranged to decontaminate a vessel which can be filter 13, syringe 40, vial 26, connector 15, or infusion line 17. Bottom housing 161b is arranged to wipe a surface of a vessel which can be filter 13, syringe 40, vial 26, connector 15, or infusion line 17. The connector 160 has a conduit formed (shown but not labeled) between the middle compartments of top housing 161a and bottom housing 161b allowing for a fluidic communication between two vessels. Housing 161a and/or 161b may also be four or five compartment (not shown) housings.

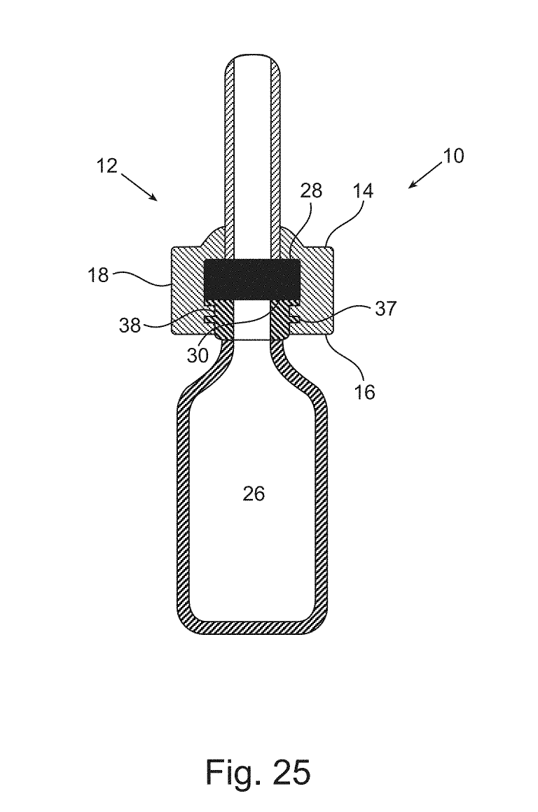

FIG. 25 shows a side cut view of system 10, wherein a vessel 26 is coupled to housing 12. The vessel 26 is shown when wiped off by wiping member 28. Wiping member 28 wipes off the entire surface 30 of the vessel 26. Vessel 26 includes a rail 37 allowing the vessel 26 to slide along the housing 12 and within rail mechanism 38 of housing 12.

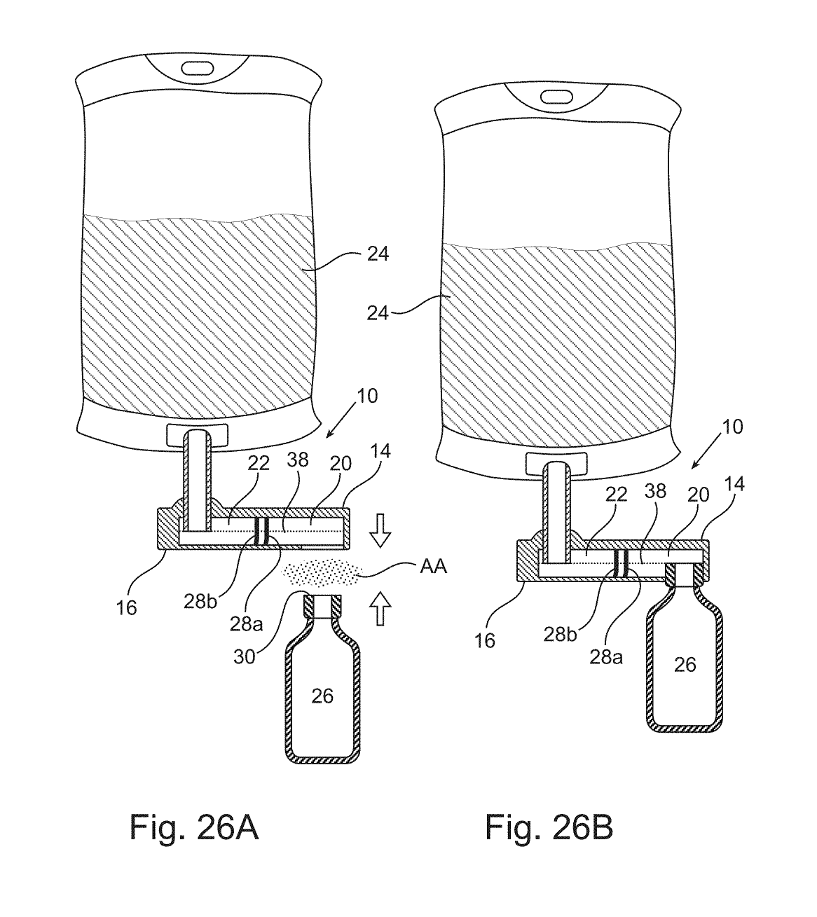

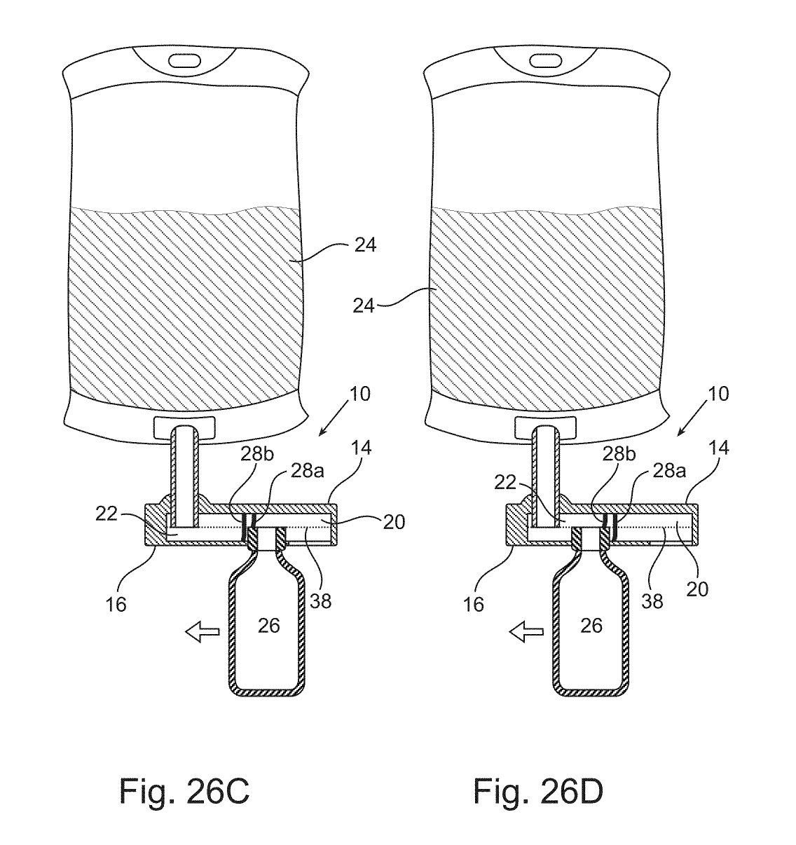

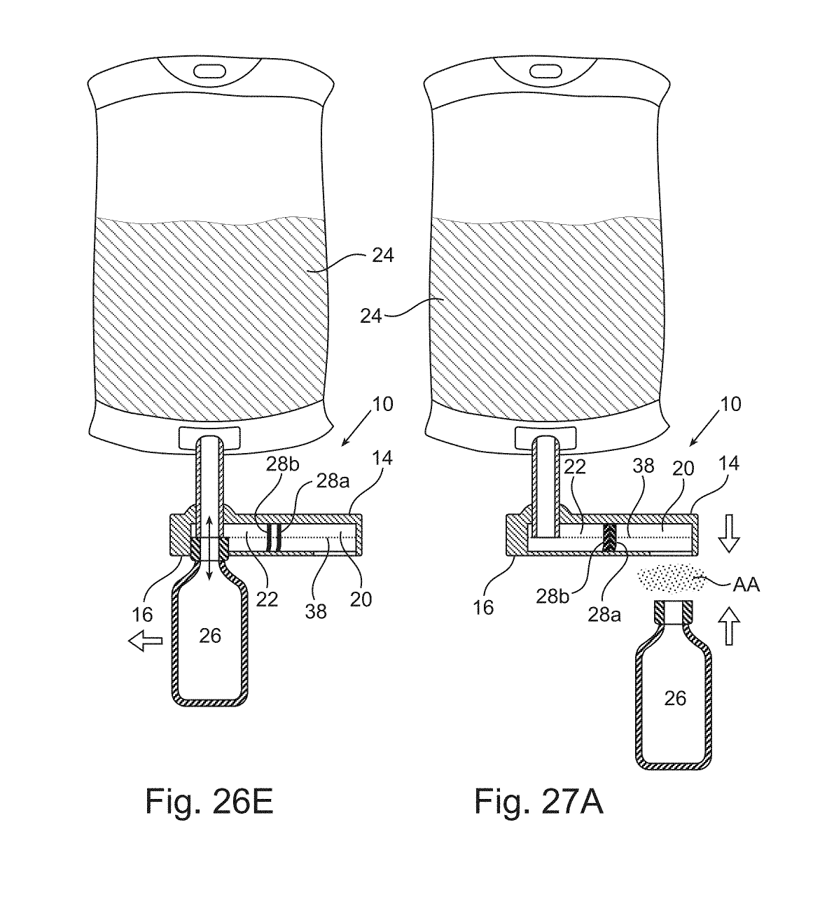

FIGS. 26A-26E demonstrate sequential steps of decontamination of vessel 26 by system 10. System 10 optionally includes a double wiper having a first wiping member 28a and a second wiping member 28b. Wiping members 28 are disposed one after the other between first compartment 20 and second compartment 22 when spaced apart by a distance D. in certain embodiments, the distance can be up to a few centimeters, or as little as a few millimeters. Wiping members 28a and 28b are configured to ensure a hermetic isolation of compartment 22 and/or decontamination of surface 30 of vessel 26. In one or more embodiments, the space/compartment between first wiping member 28a and second wiping member 28b may contain a sterilizing and/or disinfecting substance. Optionally, in one or more embodiments, the space/compartment between first wiping member 28a and second wiping member 28b may be of a higher or lower pressure than first compartment 20 and/or second compartment 22. As shown in FIG. 26C vessel 26 is firstly wiped off by wiping member 28a and while being wiped off second compartment 22 is maintained closed. FIG. 26D shows wiping member 28b that wipes off vessel 26 and wiping member 28a ensures second compartment 22 is maintained closed.

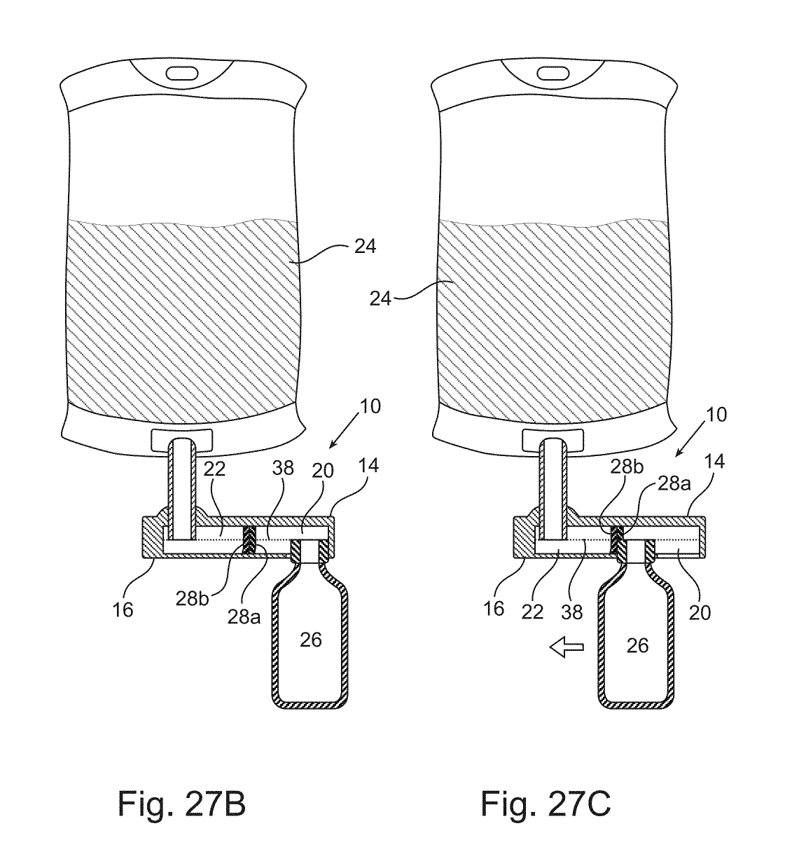

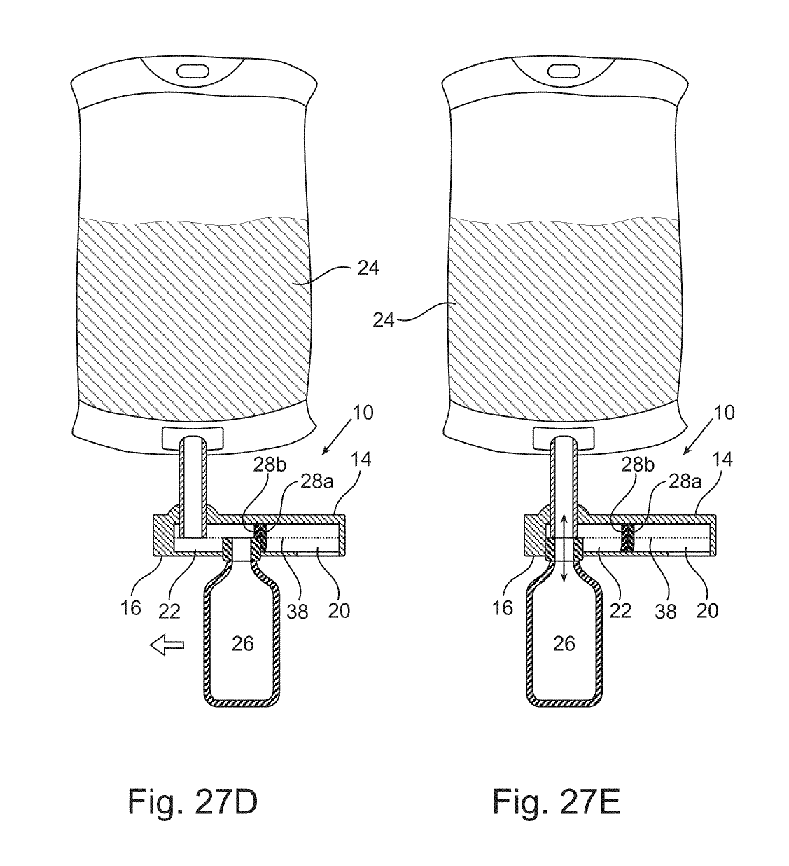

FIGS. 27A-27E demonstrate alternative embodiment of system 10 which optionally includes a double wiper having a first wiping member 28a and a second wiping member 28b wherein the wiping members 28 disposed one after the other between first compartment 20 and second compartment 22 when no space is present between the wiping members.

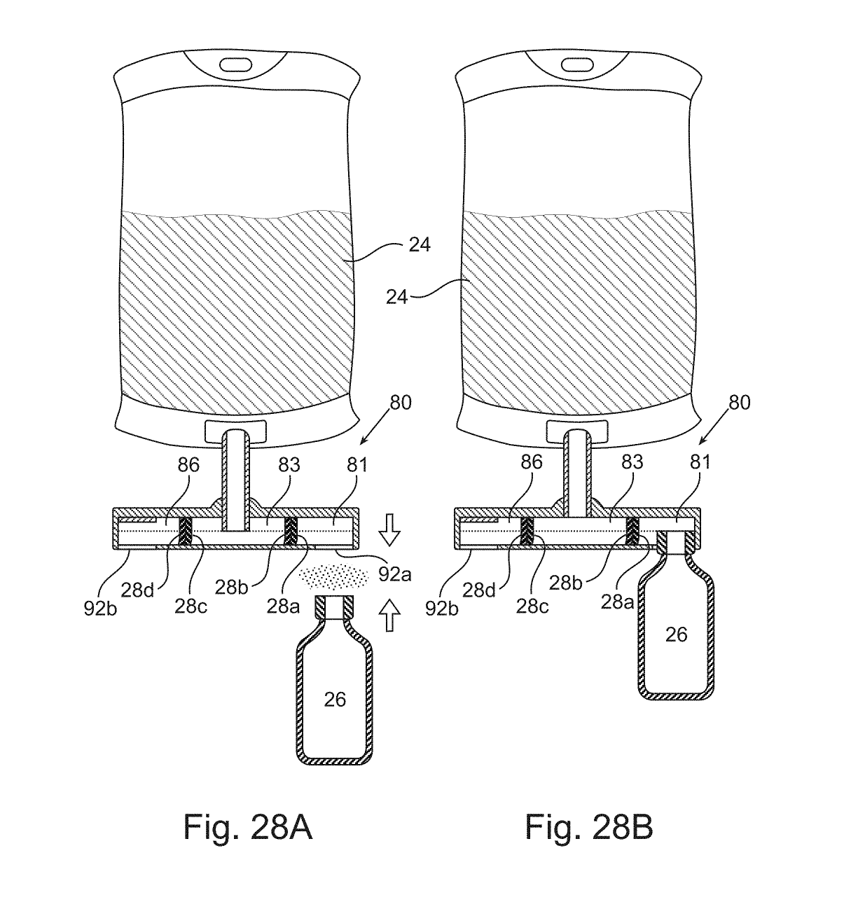

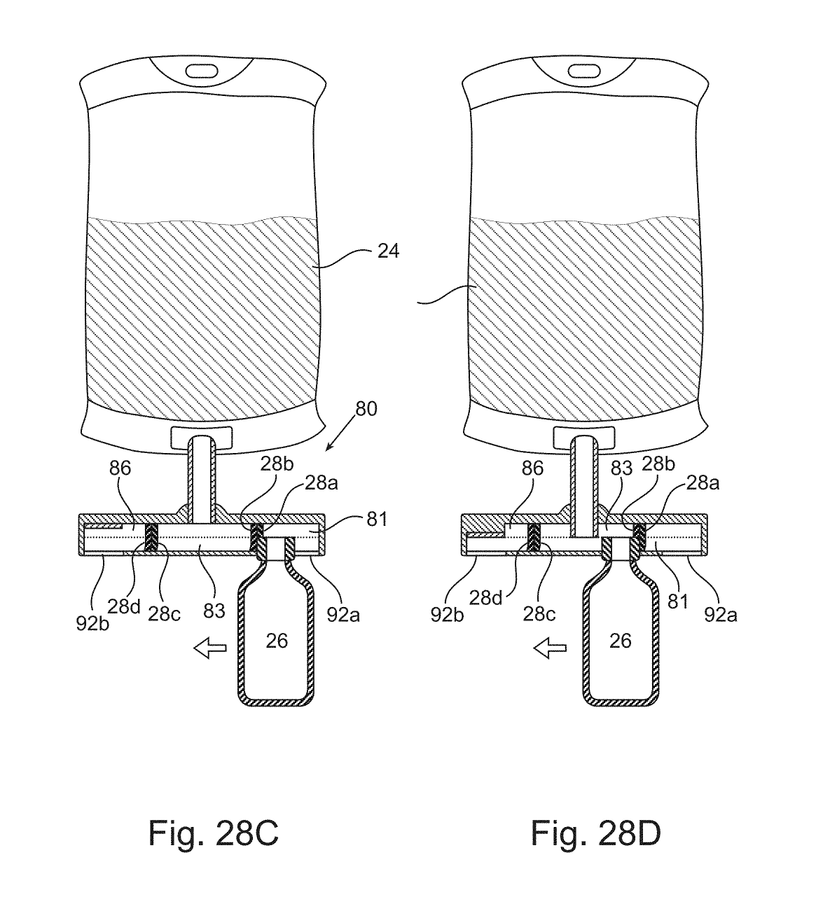

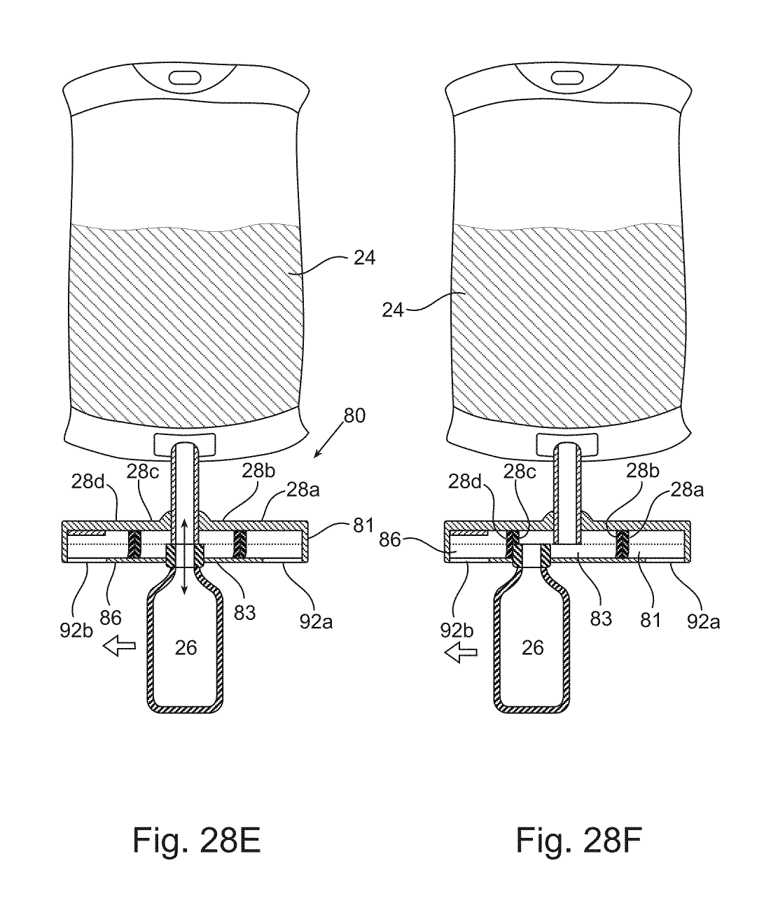

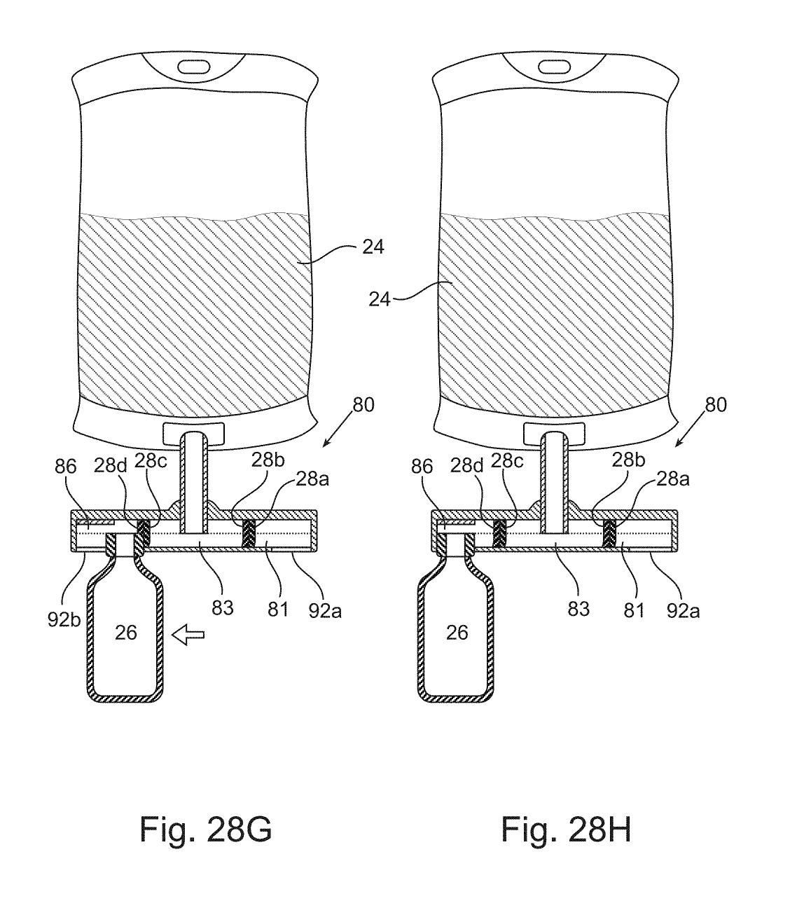

FIGS. 28A-28I demonstrate sequential steps of decontamination of vessel 26 by system 80. System 80 optionally includes a double wiper between each of the compartments. The system 80 according to this embodiment includes a first wiping member 28a and a second wiping member 28b disposed one after the other between first compartment 81 and second compartment 83. A third wiping member 28c and a fourth wiping member 28d disposed one after the other between second compartment 83 and third compartment 86. No space is present between the wiping members such that the wiping members are in close contact with each other.

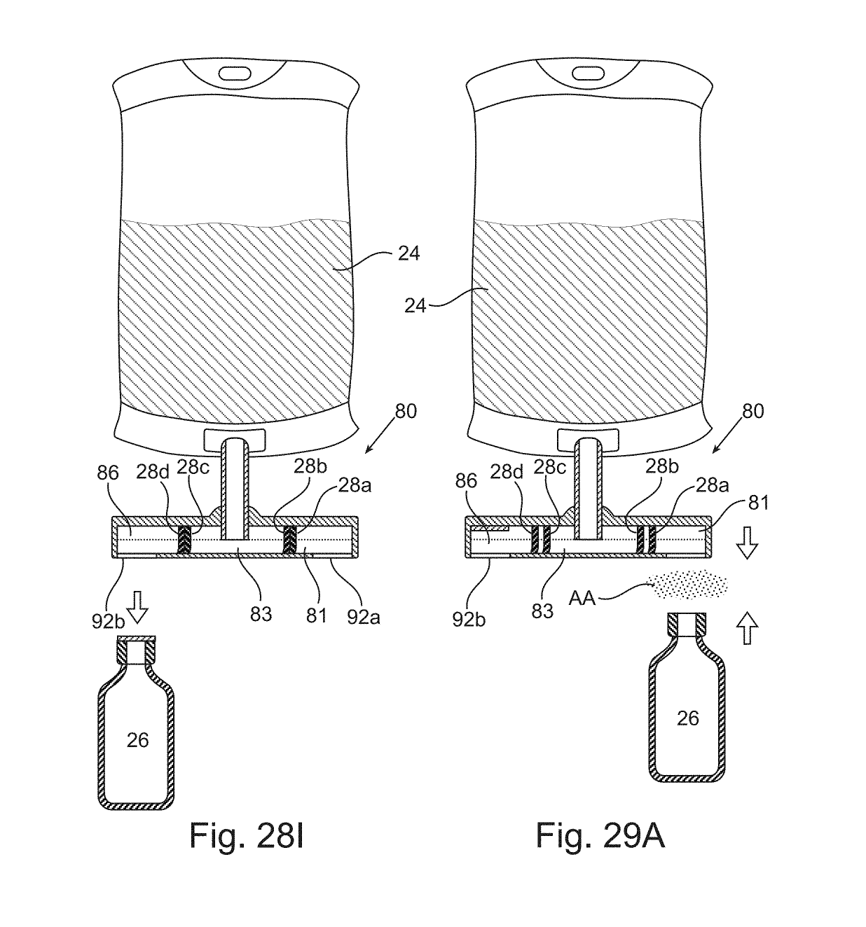

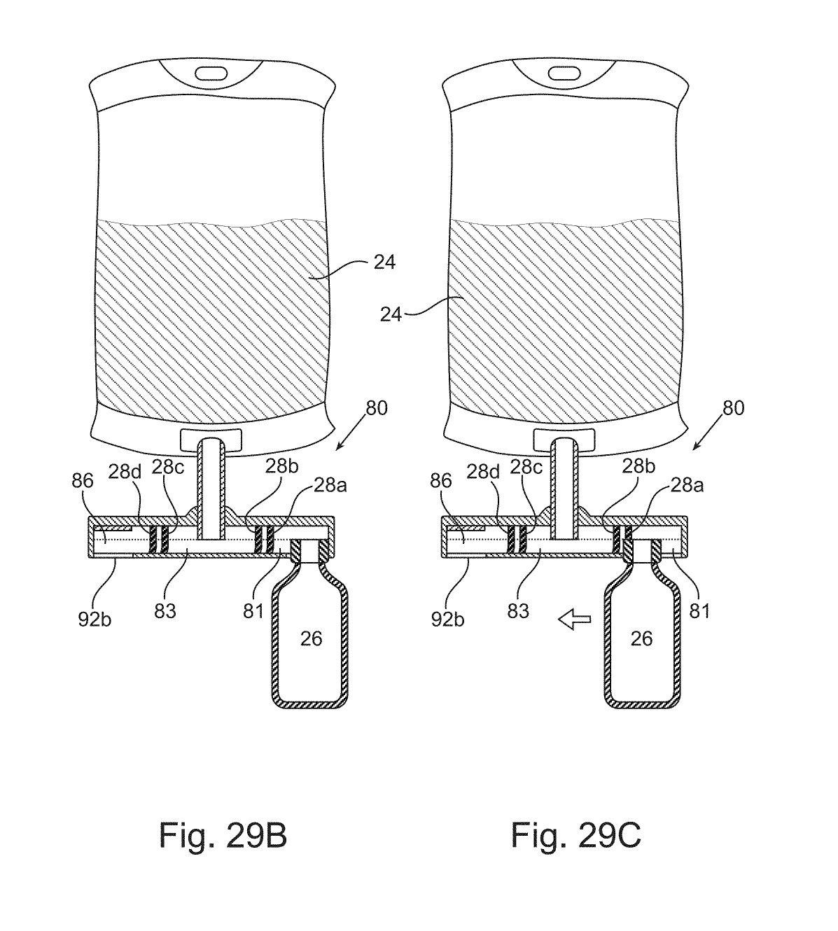

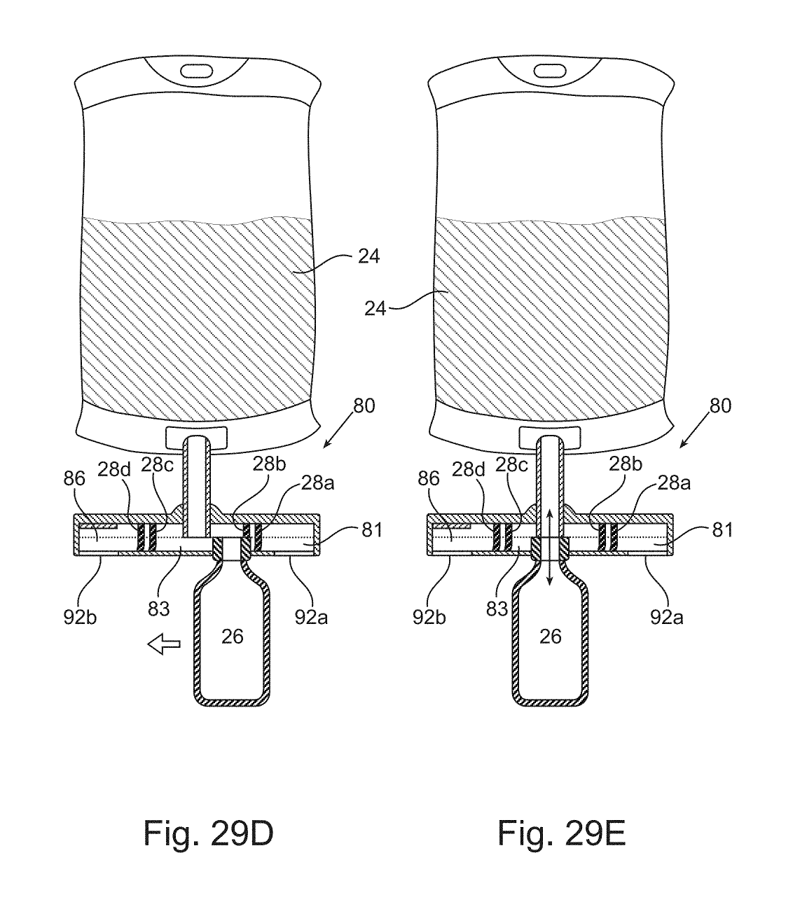

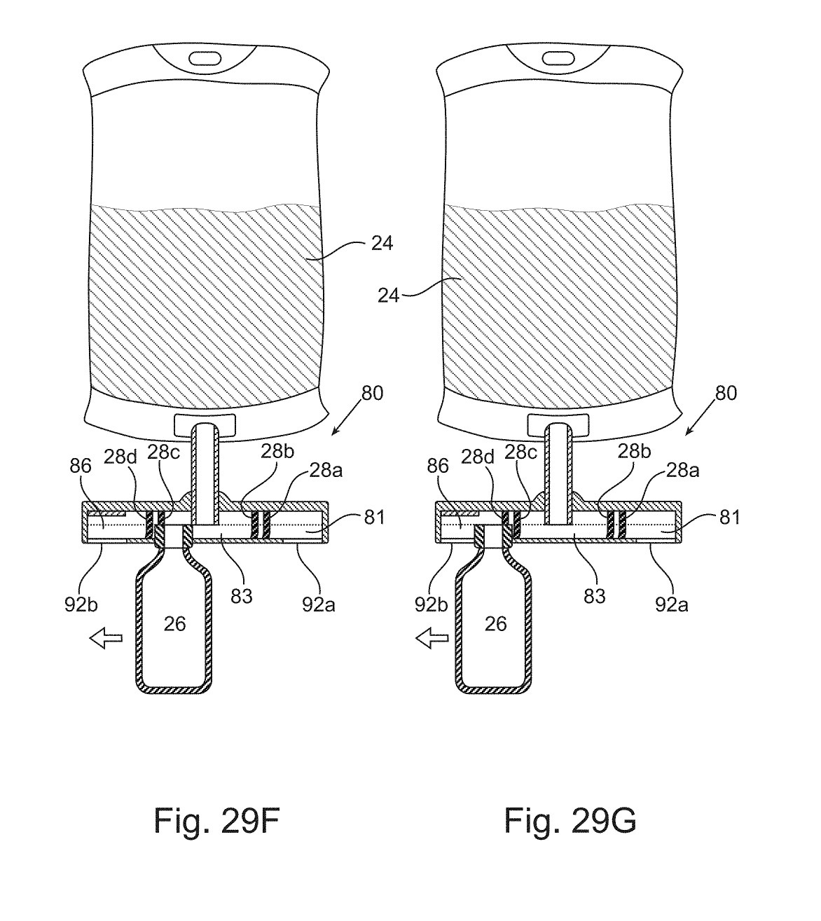

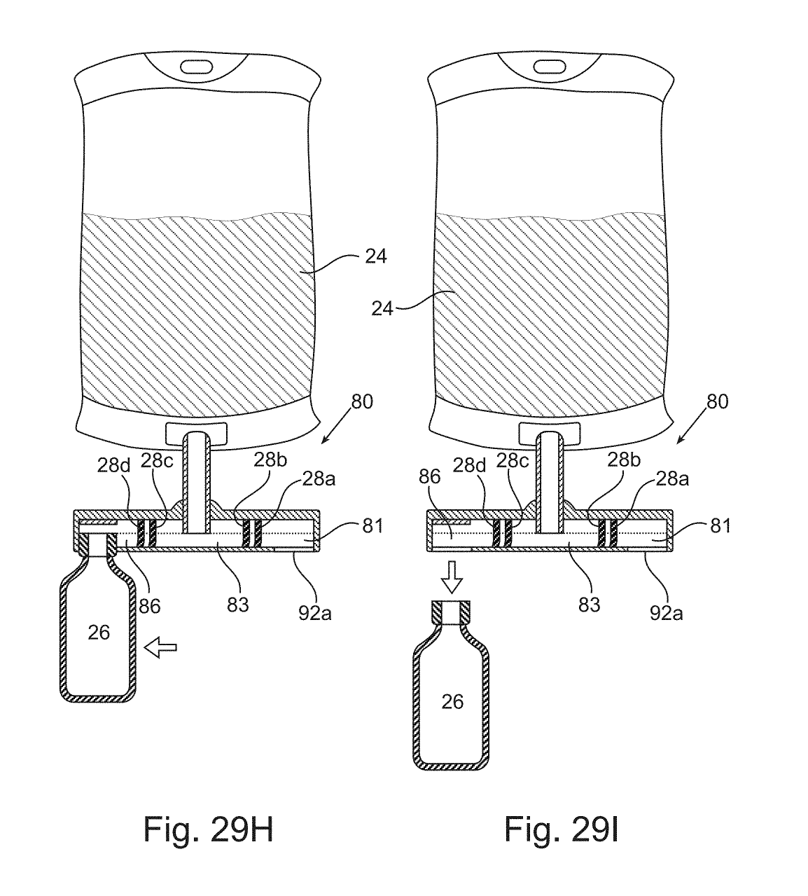

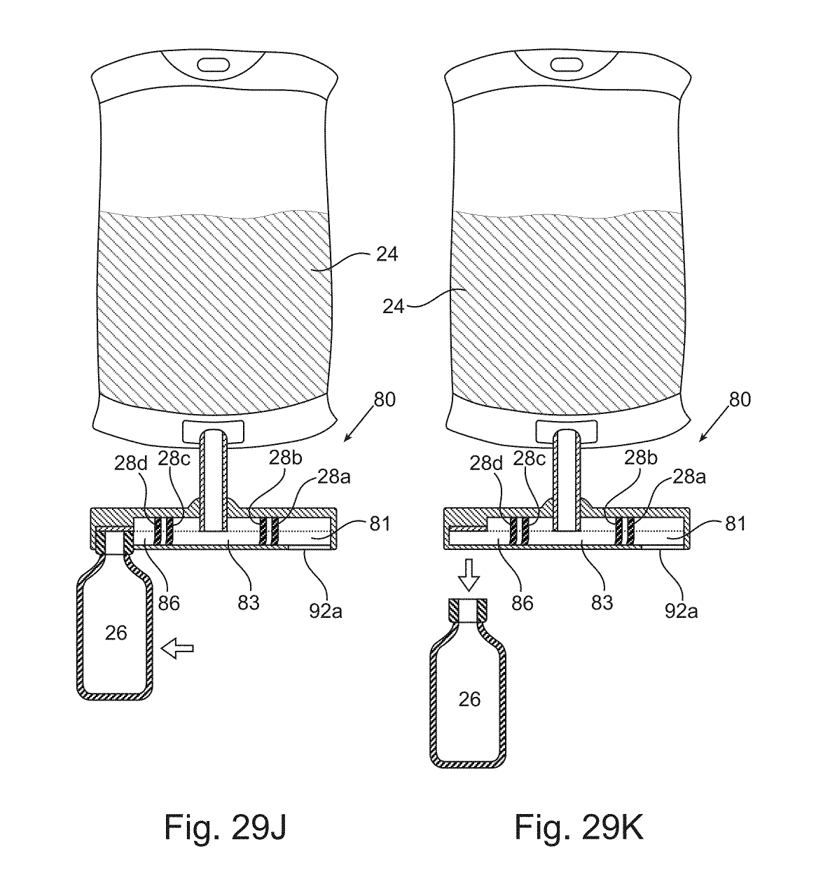

FIGS. 29-29K demonstrate alternative embodiment of system 80 which optionally includes a double wiper between each two compartments when spaced apart such that a gap X having a distance of a millimeter or less or up to a few centimeters is present between two wiping members. The system 80 according to this embodiment includes a first wiping member 28a and a second wiping member 28b being spaced apart and disposed one after the other between first compartment 81 and second compartment 83. A third wiping member 28c and a fourth wiping member 28d being spaced apart and disposed one after the other between second compartment 83 and third compartment 86.

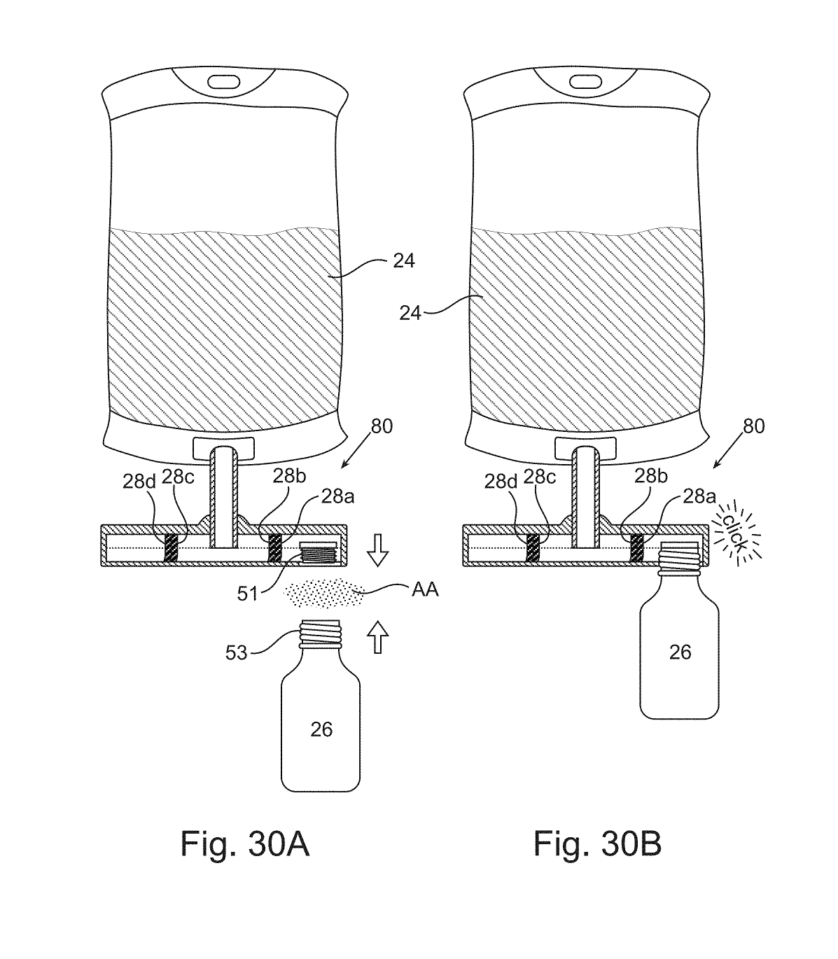

FIGS. 30A-30B demonstrate system 80 with two pairs of double wipers 28 that are in contact. The system includes an engagement mechanism in the form of thread 51 and complementary thread 53. In one or more embodiments, a click sound may be produced upon engagement of thread 53 of vial 26 to thread 51 of system 80. In one or more embodiments, the click sound may alert a user that vial 26 has engaged system 80. In one or more embodiments, other sounds may be produced.

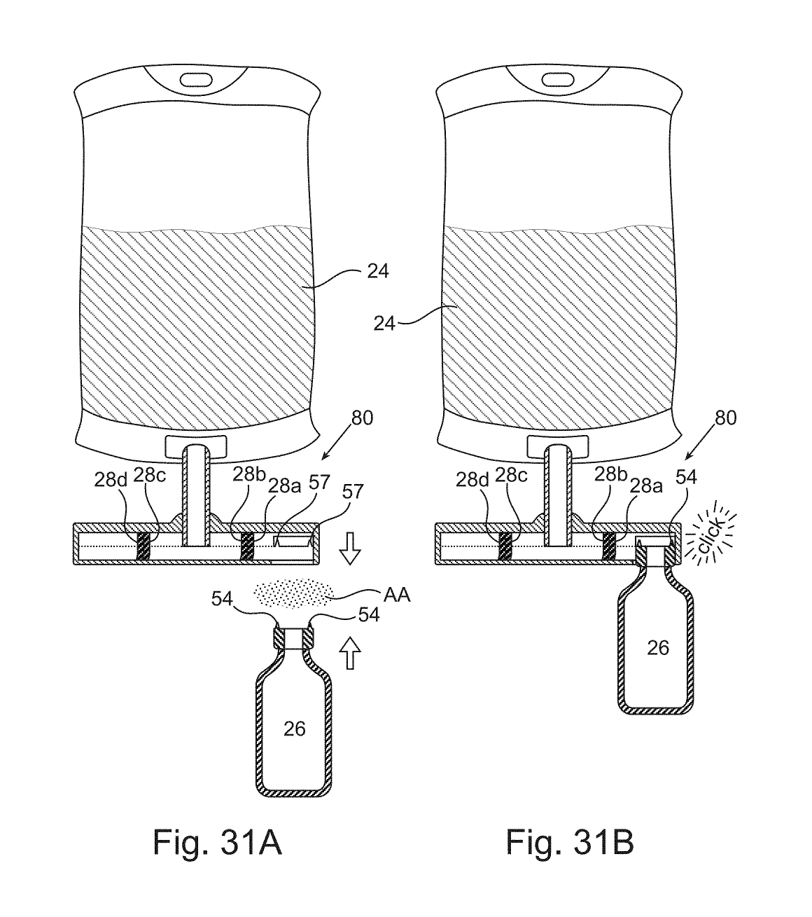

FIGS. 31A-31B demonstrate an alternative embodiment of system 80 with two pairs of double wipers 28 that are in contact. The system includes an engagement mechanism in the form of ratchet teeth 54 and 57. In one or more embodiments, a click sound may be produced upon engagement of vial 26 to ratchet teeth 57. In one or more embodiments, the click sound may alert a user that vial 26 has engaged system 80. In one or more embodiments, other sounds may be produced.

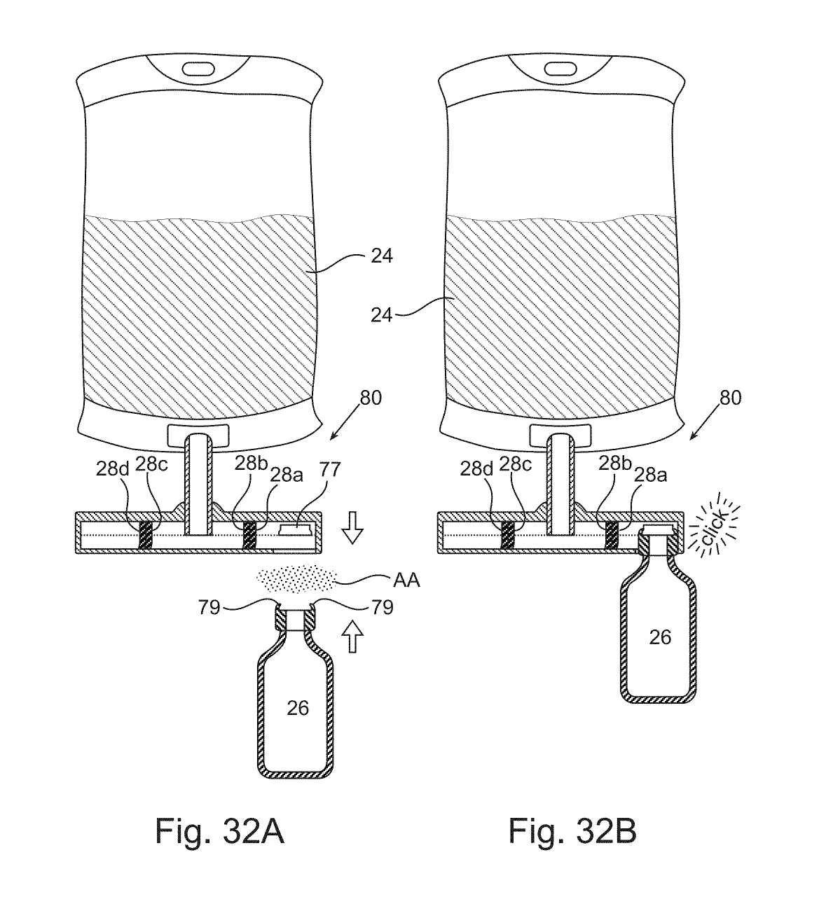

FIGS. 32A-32B demonstrate an alternative embodiment of system 80 with two pairs of double wipers 28 that are in contact. The system includes an engagement mechanism in the form of fasteners 79 configured to engage a retention member 77 housed in or on the housing. Optionally, the retention member 77 may be attached, integrally attached, and/or disposed on a rail (horizontal dotted line inside the housing of system 80. A click sound may be produced upon or at about the time of engagement between fasteners 79 and the retention member 77 to alert a user that engagement has occurred. In one or more embodiments, other sounds may be produced.

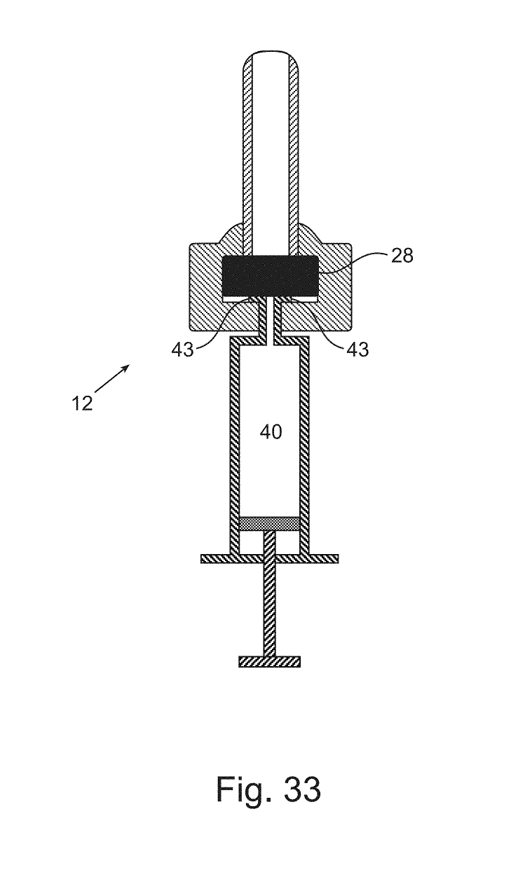

FIG. 33 demonstrates a side cut view showing a syringe 40 which further includes wings 43 extending from the surface of the syringe's port and allowing the syringe 40 to engage with housing 12 and slide there along, optionally via sliding mechanism provided within housing 12. In an embodiment of the invention, wings 43 are configured to engage with a sliding mechanism and thereby slide along housing 12. Wiping member 28 is shown wiping off a surface of the syringe as the wings 43 of syringe 40 are engaged with housing 12. In one or more embodiments, wings 43 of syringe 40 may engage a rail mechanism of the housing. In one or more embodiments, wings 43 may be present any vessel, container, and/or medical device.

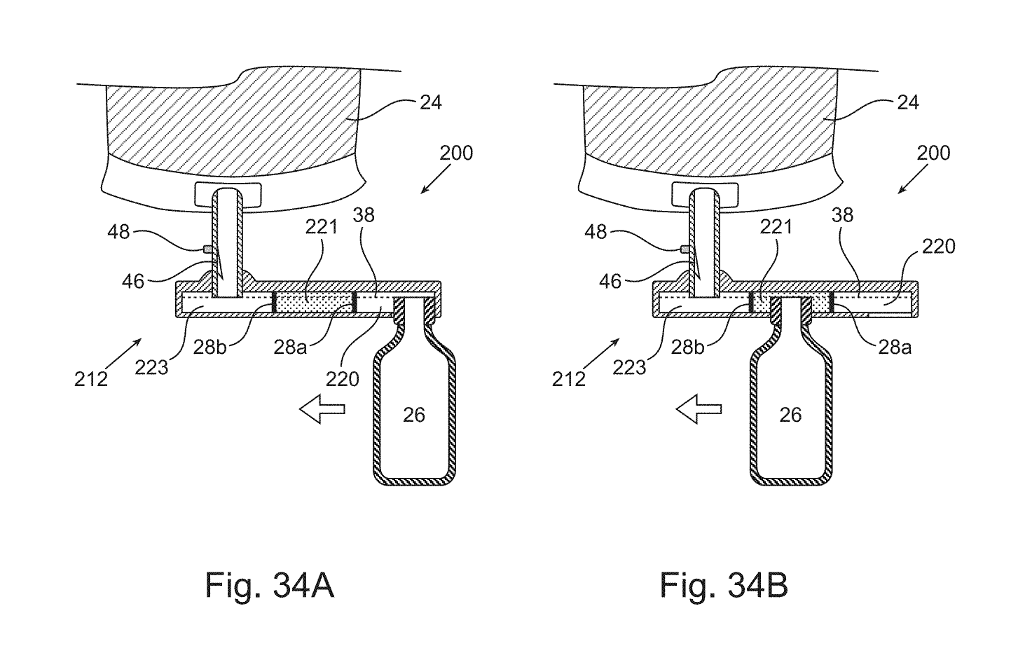

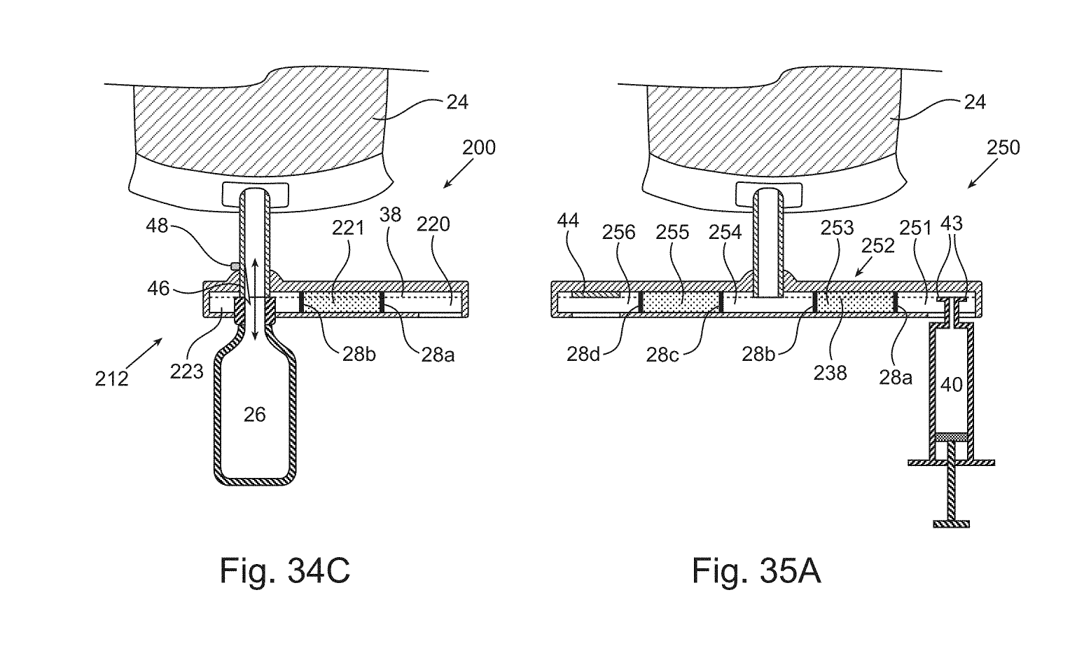

FIGS. 34A-34C demonstrate system 200 which includes a housing 212 with first non-sterile compartment 220 configured to receive vessel 26, a second decontaminating compartment 221 that includes a sterilizing substance, and a third compartment 223 for fluid transfer between vessels. In FIG. 34A, vessel 26 is shown within first compartment 220. The vessel 26 then slides along rail 38 towards second compartment 221 and passes through wiping member 28a which decontaminates a surface of the vessel 26. In FIG. 34B, vessel 26 is further decontaminated by the sterilizing substance present within compartment 221. The sterilizing substance may be a liquid such as, but not limited to, isopropyl alcohol, ethanol, hydrogen peroxide or the alike. Optionally, compartment 221 may contain an absorptive or sponge-like material saturated with the sterilizing substance. In FIG. 34C, vessel reaches compartment 223 after passing through second wiper member 28b. A fluid passageway can then be established between vessel 26 and vessel 24 upon piercing a surface or cover on top of vessel 26 by piercing member 46 and piercing member actuator 48. Optionally, a fluid passageway can be established between vessel 26 and vessel 24 upon the breaking/rupturing of a frangible seal covering the aperture/opening (not shown) of vessel 26 and/or vessel 24. Second compartment 221 may have a pressure greater than first non-sterile compartment 220. Second compartment 221 may have a pressure greater than third compartment 223. Optionally, second compartment 221 may have a pressure greater than ambient air or atmospheric pressure. Optionally, third compartment 223 may have a pressure greater than second compartment 221. Optionally, third compartment 223 may have a pressure greater than ambient air or atmospheric pressure. Optionally, second compartment 221 may have a pressure less than first non-sterile compartment 220. Optionally, second compartment 221 may have a pressure less than third compartment 223. Optionally, second compartment 221 may have a pressure less than ambient air or atmospheric air. Optionally, third compartment 223 may have a pressure less than second compartment 221. Optionally, third compartment may have a pressure less than ambient air or atmospheric air.

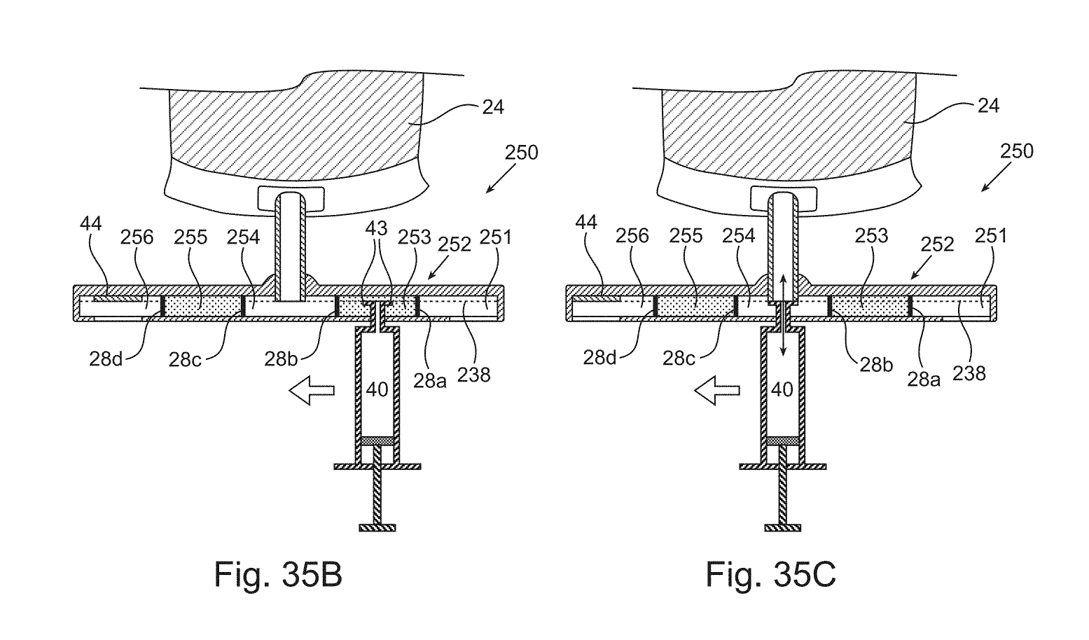

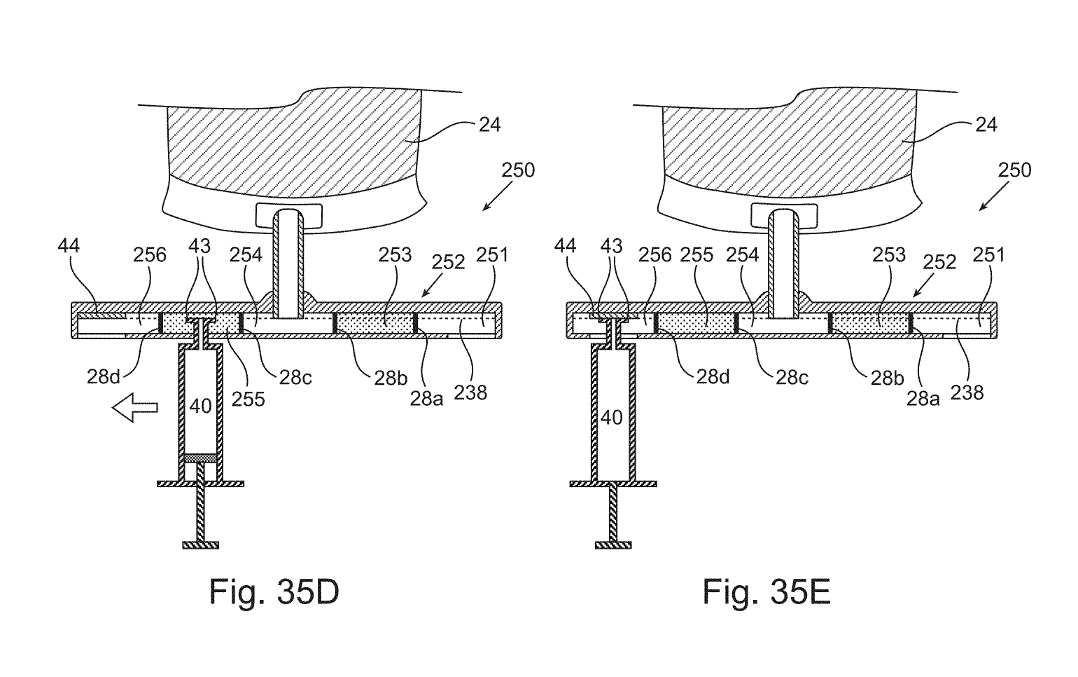

FIGS. 35A-35E demonstrate yet another system 250 which includes housing 252 with first non-sterile compartment 251, a second decontaminating compartment 253, a third compartment 254, a fourth compartment 255 for decontaminating a vessel, and a fifth compartment 256. The system 250 further optionally includes a plurality of wiping members 28. In an embodiment of the invention, the system includes at least one wiping member 28, at least two, at least three or at least four wiping members 28. In FIG. 35A, vessel 40 is shown within first compartment 251. The vessel 40 then slides along rail 238 towards second compartment 253 and passes through wiping member 28a which decontaminates a surface of the vessel 40. In FIG. 35B, vessel 40 is further decontaminated by the sterilizing substance present within compartment 253. In FIG. 35C vessel 40 reaches compartment 254 after passing through second wiper member 28b. A fluid passageway can then be established between vessel 40 and vessel 24. In FIG. 35D vessel continues to slide along housing 252 towards fourth compartment 255 via wiping member 28c. Fourth compartment 255 which further includes a sterilizing substance further decontaminates the surface of syringe 40 from any fluid residuals. At FIG. 35E, vessel 40 reaches fifth compartment 256 and on the way passes through wiping member 28d. The vessel 40 can be resealed by a cover such as displaceable member 44 within compartment 256. In one or more embodiments, any of compartments 251, 253, 254, 255 and 256 may house a disinfection and/or sterilizing substance. In one or more embodiments, any of compartments 251, 253, 254, 255 and 256 may have a pressure greater than or less than ambient air and/or atmospheric pressure. In one or more embodiments, any of compartments 251, 253, 254, 255 and 256 may have a pressure greater than or less than an adjacent compartment.

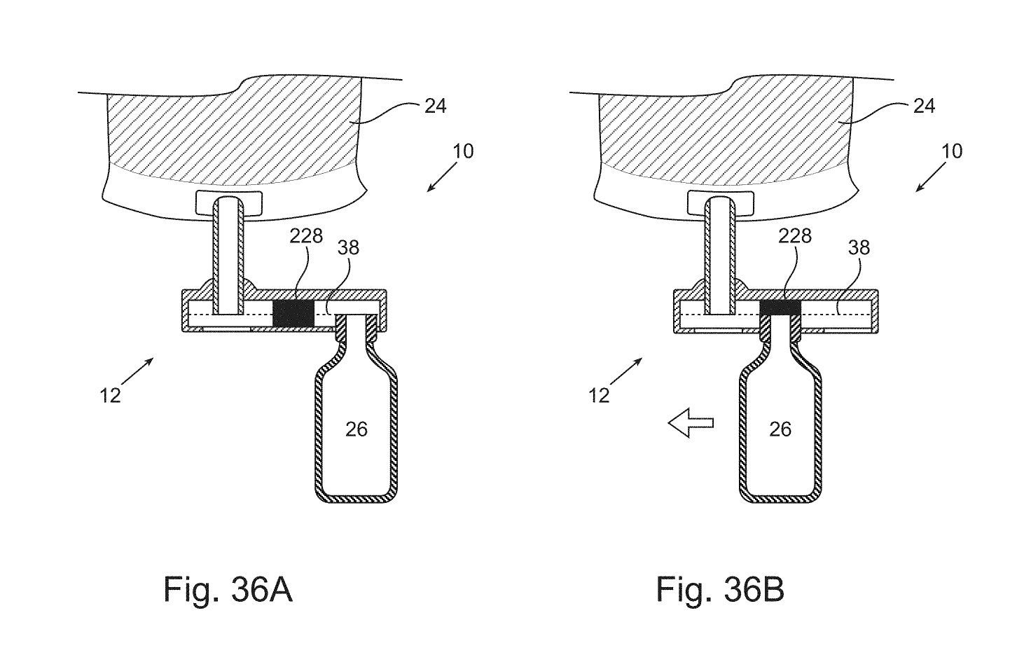

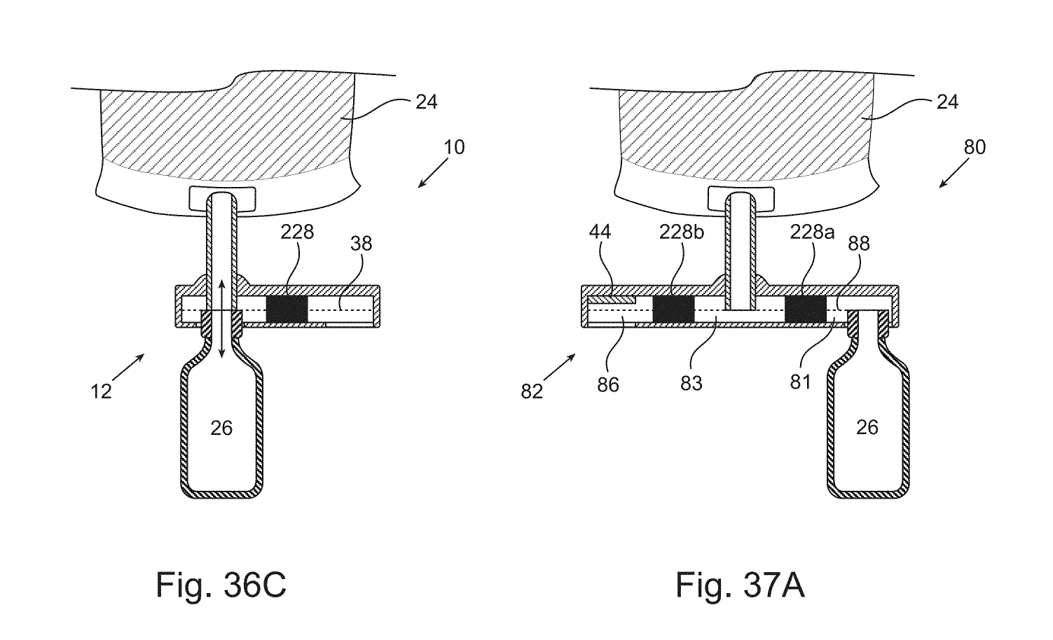

FIGS. 36A-36C demonstrate system 10 which includes a wiping member 228 having wide dimensions. Optionally, the wiping member 228 is at least as wide or wider than a typical port of a vessel such as vial 26. Optionally, wiping member 228 is having wide width and/or length. The wiping member 228 is configured to clean off the entire surface of vial 26.

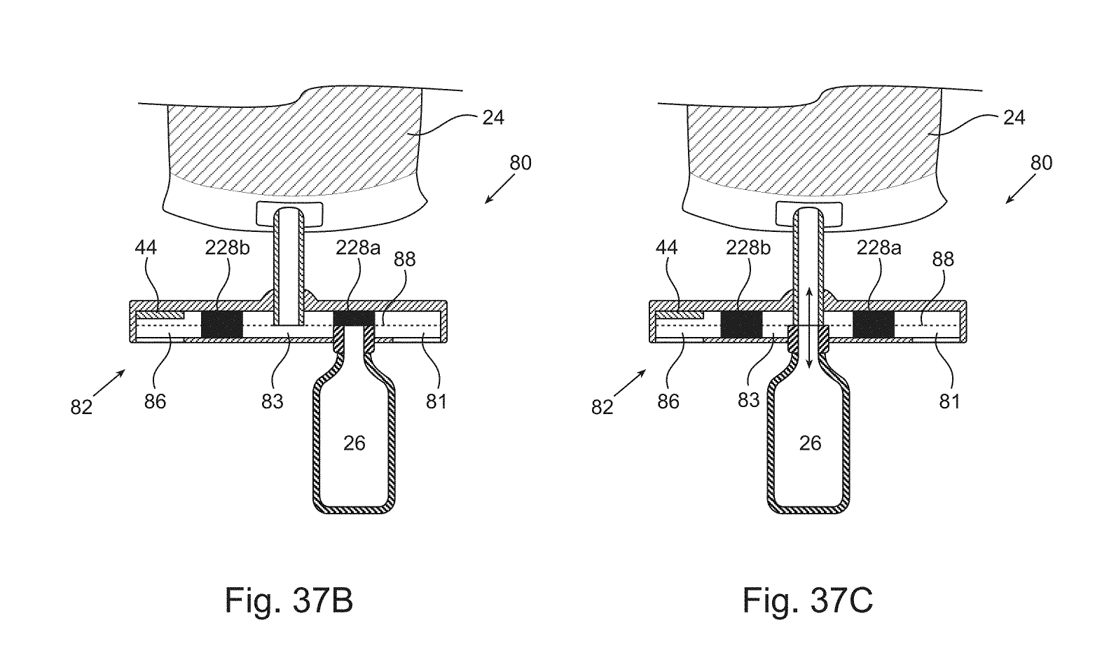

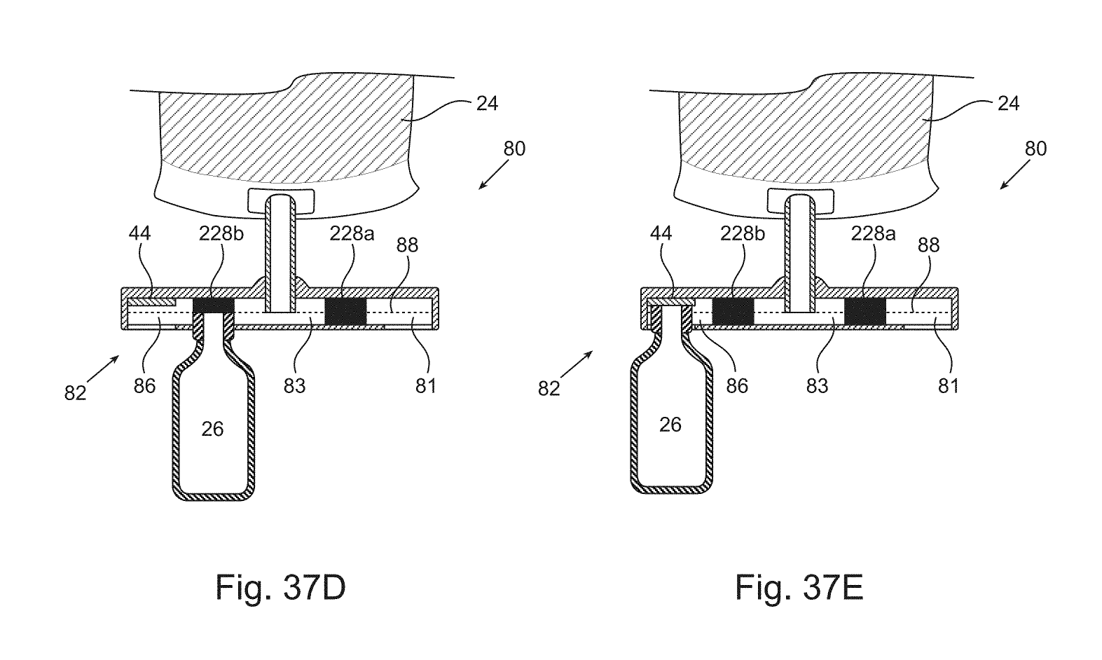

FIGS. 37A-37E demonstrate system 80 which includes a double wiper having wiping members 228a and 228b having wide dimensions. The system 80 includes at third compartment 86 having a resealing mechanism having and/or displaceable member 44 disposed in compartment 86, such that the vessel 26 may be resealed by displaceable member 44 within compartment 86 about the time or prior to detaching from housing 82. In one or more embodiments, system 80 further comprises a detachment safety mechanism (not shown) configured to prevent detachment of vessel 26 from compartment 86 prior to resealing vessel 26 by displaceable member 44. Detachment safety mechanism (not shown) prevents potential leakage of fluid from within system 80 to an exterior of system 80. Detachment safety mechanism (not shown) also prevents recontamination of a beneficial substance that may be housed in vessel 26.

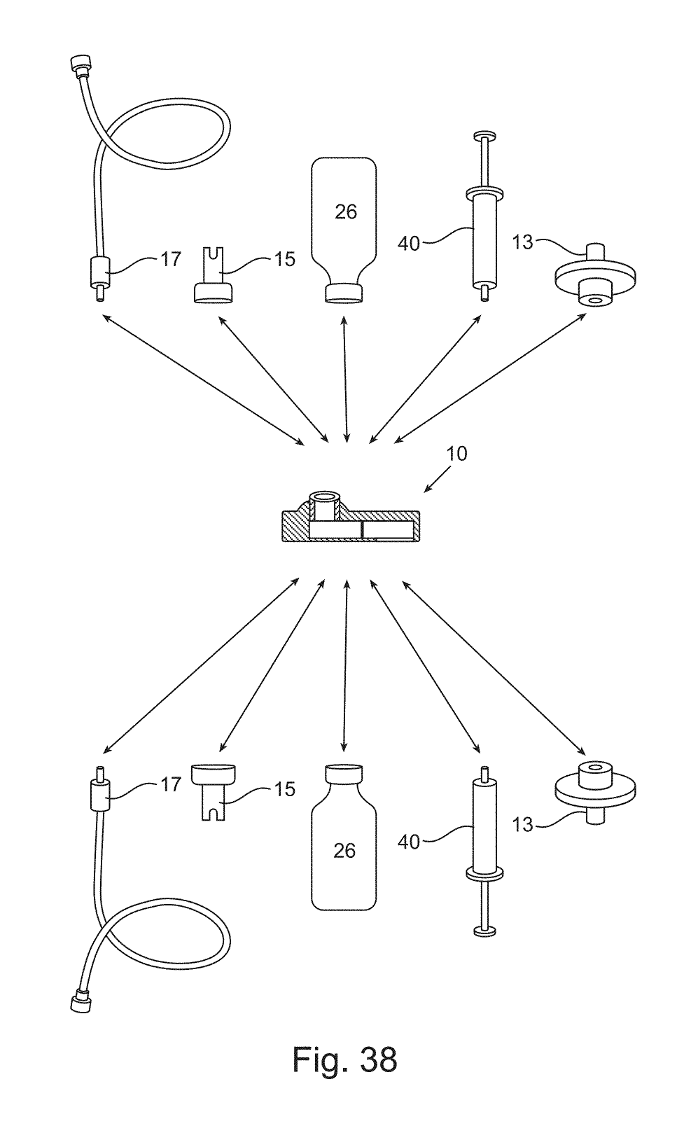

FIG. 38 demonstrates device 10 which is capable of being coupled to various types of medical vessels. The device 10 may be provided when already coupled with first medical vessel. Alternatively, the device 10 can be provided as a separate unit not connected to any medical vessel. Optionally, a first medical vessel can be coupled with device 10 in a sterile manner, optionally, within a sterile chamber or room. Optionally, a first medical vessel can be coupled and/or integrally attached and/or manufactured with device 10. Optionally, a first medical vessel can form a unitary structure with device 10 and be provided to consumers as such. The device 10 is further configured to be coupled to various types of second medical vessel. The first and/or second medical vessel may be selected from a filter 13, a syringe 40, a vial 26, a connector 15, and an infusion line 17. Other vessels are also contemplated such as a manifold, a connector, a bag, etc.

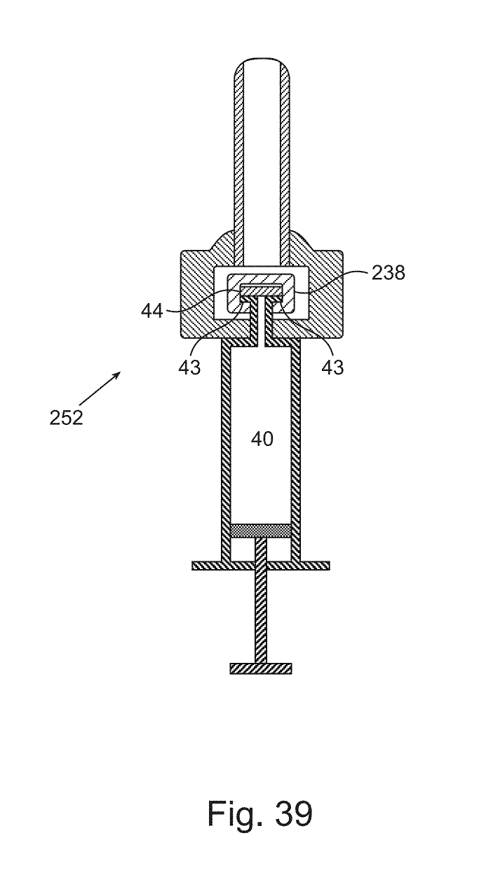

FIG. 39 is a side view of system 252. Syringe 40 is shown when resealed by displaceable member 44 and when connected to rail 238 via syringe 40. The displaceable member 44 may be a cap, a cover or a seal. The cap, cover or seal may be temporarily or permanently affixed to syringe 40. The displaceable member 44 may be rigid, flexible or made of a frangible material.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device for the decontamination of a surface of a vessel, comprising:
a housing having a top, a bottom, and at least one opening configured to engage at least one vessel;
at least one wiping member disposed within the housing;
a sliding mechanism configured to provide for the sliding of the vessel surface from a first compartment of the housing to at least a second compartment of the housing,
wherein when the at least one vessel engages the at least one opening of the vessel, the at least one vessel slides along the sliding mechanism of the device from a first position to at least a second position.

2. The system of claim 1, further comprising a third compartment.

3. The system of claim 2, wherein the third compartment includes a resealing mechanism for resealing the at least one vessel with a cap, a seal or a cover.

4. The system of claim 3, wherein when the at least one vessel slides from the second position to the third position, the at least one vessel interacts with the resealing mechanism to reseal the at least one vessel.

5. The system of claim 1, wherein the at least one wiping mechanism is located between the first compartment and the second compartment.

6. The system of claim 2, further comprising a second wiping member located between the second compartment and the third compartment.

7. The system of claim 1, wherein the second compartment includes a sterilizing/disinfecting substance.

8. The system of claim 2, wherein the third compartment includes a sterilizing/disinfecting substance.

9. The system of claim 2, further comprising a fourth compartment and a fifth compartment.

10. The system of claim 9, further comprising a third wiping member located between the third compartment and the fourth compartment and a fourth wiping member is located between the fourth compartment and the fifth compartment.

11. The system of claim 10, wherein the fifth compartment includes a resealing mechanism for resealing the at least one vessel with a cap, a seal or a cover.

12. The system of claim 3, wherein the resealing member is disposed within the housing.

13. A device for the decontamination of a surface of a vessel, comprising:
a housing having a top and a bottom;
at least one wiping member disposed within the housing;
at least one opening within the housing configured to engage at least one vessel; and
a hinge mechanism configured to provide for the sliding of the vessel surface from a first compartment of the housing to at least a second compartment of the housing,
wherein when the at least one vessel engages the at least one opening of the vessel, the at least one vessel may slide along the sliding mechanism of the device from a first position to at least a second position.

14. The device of claim 12, further comprising a third compartment.

15. The device of claim 13, wherein the third compartment includes a resealing mechanism for resealing the at least one vessel with a cap, a seal or a cover.

16. The device of claim 14, wherein when the at least one vessel slides from the second position to the third position, the at least one vessel interacts with the resealing mechanism to reseal the at least one vessel.

17. The device of claim 12, wherein the at least one wiping mechanism is located between the first compartment and the second compartment.

18. The device of claim 13, wherein a second wiping member is located between the second compartment and the third compartment.

19. The device of claim 12, wherein the second compartment includes a sterilizing/disinfecting substance.

20. The device of claim 13, wherein the third compartment includes a sterilizing/disinfecting substance.

21. The device of claim 13, wherein the first compartment houses a sterilizing/disinfecting substance and an aperture of the first compartment is sealed.
